SEQUENCE LISTING (208 sequences)

SEQ ID NO: 1 - protein, 258 aa, Artificial Sequence
  Synthetic
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 54 aa, Artificial Sequence
  Synthetic
MLQMAGQCSQNEYFDSLLHACIPCQLRCSSNTPPLTCQRYCNASVTNSVKGTNA

SEQ ID NO: 3 - protein, 124 aa, Artificial Sequence [sequence omitted]

SEQ ID NO: 4 - protein, 422 aa, Artificial Sequence
  Synthetic
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 5 - protein, 120 aa, Artificial Sequence
  Synthetic
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 6 - protein, 150 aa, Artificial Sequence [sequence omitted]

SEQ ID NO: 7 - protein, 220 aa, Artificial Sequence
  Synthetic
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 8 - protein, 204 aa, Artificial Sequence
  Synthetic
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 9 - protein, 651 aa, Artificial Sequence
  Synthetic
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 10 - protein, 104 aa, Artificial Sequence
  Synthetic
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 11 - protein, 192 aa, Artificial Sequence
  Synthetic
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 12 - protein, 112 aa, Artificial Sequence
  Synthetic
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 13 - protein, 112 aa, Artificial Sequence
  Synthetic
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 14 - protein, 112 aa, Artificial Sequence [sequence omitted]

SEQ ID NO: 15 - protein, 206 aa, Artificial Sequence
  Synthetic
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 16 - protein, 159 aa, Artificial Sequence
  Synthetic
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - protein, 297 aa, Artificial Sequence
  Synthetic
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 18 - protein, 261 aa, Artificial Sequence
  Synthetic
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 19 - protein, 415 aa, Artificial Sequence
  Synthetic
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 20 - protein, 448 aa, Artificial Sequence [sequence omitted]

SEQ ID NO: 21 - protein, 630 aa, Artificial Sequence
  Synthetic
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 22 - protein, 214 aa, Artificial Sequence
  Synthetic
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 23 - protein, 239 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - protein, 7 aa, Artificial Sequence
  Synthetic
HHHHHHH

SEQ ID NO: 25 - protein, 8 aa, Artificial Sequence
  Synthetic
DYKDDDDK

SEQ ID NO: 26 - protein, 9 aa, Artificial Sequence
  Synthetic
YPYDVPDYA

SEQ ID NO: 27 - protein, 10 aa, Artificial Sequence
  Synthetic
EQKLISEEDL

SEQ ID NO: 28 - protein, 8 aa, Artificial Sequence
  Synthetic
WSHPQFEK

SEQ ID NO: 29 - protein, 15 aa, Artificial Sequence
  Synthetic
GLNDIFEAQKIEWHE

SEQ ID NO: 30 - protein, 220 aa, Artificial Sequence
  Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 123 aa, Artificial Sequence
  Synthetic
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 32 - protein, 36 aa, Artificial Sequence
  Synthetic
MVKVIGRRSLGVQRIFDIGLPQDHNFLLANGAIAAN

SEQ ID NO: 33 - protein, 105 aa, Artificial Sequence
  Synthetic
CISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQ

SEQ ID NO: 34 - protein, 48 aa, Artificial Sequence
  Synthetic
SPEIEKLSQSDIYWDSIVSITETGVEEVFDLTVPGPHNFVANDIIVHN

SEQ ID NO: 35 - protein, 119 aa, Artificial Sequence
  Synthetic
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFAR

SEQ ID NO: 36 - protein, 79 aa, Artificial Sequence
  Synthetic
GKPEFAPTTYTVGVPGLVRFLEAHHRDPDAQAIADELTDGRFYYAKVASVTDAGVQPVYSLRVDTADHAFITNGFVSHN

SEQ ID NO: 37 - protein, 121 aa, Artificial Sequence
  Synthetic
SVDYNEPIIIKENGEIKVVKIGELIDKIIENSENIRREGILEIAKCKGIEVIAFNSNYKFKFMPVSEVSRHPVSEMFEIVVEGNKKVRVTRSHSVFTIRDNEVVPIRVDELKVGDILVLAK

SEQ ID NO: 38 - protein, 44 aa, Artificial Sequence
  Synthetic
NSDFIFLKIKEINKVEPTSGYAYDLTVPNAENFVAGFGGFVLHN

SEQ ID NO: 39 - protein, 142 aa, Artificial Sequence
  Synthetic
CVSGDTPVLLDAGERRIGDLFMEAIRPKERGEIGQNEEIVRLHDSWRIYSMVGSEIVETVSHAIYHGKSNAIVNVRTENGREVRVTPVHKLFVKIGNSVIERPASEVNEGDEIAWPSVSENGDSQTVTTTLVLTFDRVVSKE

SEQ ID NO: 40 - protein, 31 aa, Artificial Sequence
  Synthetic
MHSGVFDVYDLMVPDYGYNFIGGNGLIVLHN

SEQ ID NO: 41 - protein, 11 aa, Artificial Sequence
  Synthetic
CVSGETPVYLA

SEQ ID NO: 42 - protein, 175 aa, Artificial Sequence
  Synthetic
DGKTIKIKDLYSSERKKEDNIVEAGSGEEIIHLKDPIQIYSYVDGTIVRSRSRLLYKGKSSYLVRIETIGGRSVSVTPVHKLFVLTEKGIEEVMASNLKVGDMIAAVAESESEARDCGMSEECVMEAEVYTSLEATFDRVKSIAYEKGDFDVYDLSVPEYGRNFIGGEGLLVLHN

SEQ ID NO: 43 - protein, 88 aa, Artificial Sequence
  Synthetic
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 44 - protein, 37 aa, Artificial Sequence
  Synthetic
MMLKKILKIEELDERELIDIEVSGNHLFYANDILTHN

SEQ ID NO: 45 - protein, 89 aa, Artificial Sequence
  Synthetic
CLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 46 - protein, 45 aa, Artificial Sequence
  Synthetic
MCEIFENEIDWDEIASIEYVGVEETIDINVTNDRLFFANGILTHN

SEQ ID NO: 47 - protein, 101 aa, Artificial Sequence
  Synthetic
CFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 48 - protein, 40 aa, Artificial Sequence
  Synthetic
MKFKLKEITSIETKHYKGKVHDLTVNQDHSYNVRGTVVHN

SEQ ID NO: 49 - protein, 38 aa, Artificial Sequence
  Synthetic
CFARDTEVYYENDTVPHMESIEEMYSKYASMNGELPFD

SEQ ID NO: 50 - protein, 134 aa, Artificial Sequence
  Synthetic
NGYAVPLDNVFVYTLDIASGEIKKTRASYIYREKVEKLIEIKLSSGYSLKVTPSHPVLLFRDGLQWVPAAEVKPGDVVVGVREEVLRRRIISKGELEFHEVSSVRIIDYNNWVYDLVIPETHNFIAPNGLVLHN

SEQ ID NO: 51 - protein, 4 aa, Artificial Sequence
  Synthetic
  MISC_FEATURE (4)..(4): Xaa is any amino acid
AEYX

SEQ ID NO: 52 - protein, 4 aa, Artificial Sequence
  Synthetic
  MISC_FEATURE (3)..(4): Xaa is any amino acid
SGXX

SEQ ID NO: 53 - protein, 4 aa, Artificial Sequence
  Synthetic
  MISC_FEATURE (3)..(4): Xaa is any amino acid
GSXX

SEQ ID NO: 54 - protein, 4 aa, Artificial Sequence
  Synthetic
  MISC_FEATURE (4)..(4): Xaa is any amino acid
MGGX

SEQ ID NO: 55 - protein, 4 aa, Artificial Sequence
  Synthetic
  MISC_FEATURE (3)..(4): Xaa is any amino acid
RYXX

SEQ ID NO: 56 - protein, 4 aa, Artificial Sequence
  Synthetic
  MISC_FEATURE (3)..(4): Xaa is any amino acid
TYXX

SEQ ID NO: 57 - protein, 4 aa, Artificial Sequence
  Synthetic
  MISC_FEATURE (3)..(4): Xaa is any amino acid
GKXX

SEQ ID NO: 58 - protein, 4 aa, Artificial Sequence
  Synthetic
  MISC_FEATURE (3)..(4): Xaa is any amino acid
NRXX

SEQ ID NO: 59 - protein, 4 aa, Artificial Sequence
  Synthetic
  MISC_FEATURE (4)..(4): Xaa is any amino acid
GGGX

SEQ ID NO: 60 - protein, 4 aa, Artificial Sequence
  Synthetic
  MISC_FEATURE (3)..(4): Xaa is any amino acid
DKXX

SEQ ID NO: 61 - protein, 4 aa, Artificial Sequence
  Synthetic
  MISC_FEATURE (3)..(4): Xaa is any amino acid
GYXX

SEQ ID NO: 62 - protein, 4 aa, Artificial Sequence
  Synthetic
  MISC_FEATURE (1)..(1): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (2)..(2): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (3)..(4): Xaa is any amino acid
XXXX

SEQ ID NO: 63 - protein, 4 aa, Artificial Sequence
  Synthetic
  MISC_FEATURE (1)..(1): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (2)..(2): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (3)..(3): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (4)..(4): Xaa is any amino acid
XXXX

SEQ ID NO: 64 - protein, 4 aa, Artificial Sequence
  Synthetic
  MISC_FEATURE (4)..(4): Xaa is any amino acid
CFNX

SEQ ID NO: 65 - protein, 4 aa, Artificial Sequence
  Synthetic
  MISC_FEATURE (4)..(4): Xaa is any amino acid
SVYX

SEQ ID NO: 66 - protein, 4 aa, Artificial Sequence
  Synthetic
  MISC_FEATURE (4)..(4): Xaa is any amino acid
SIEX

SEQ ID NO: 67 - protein, 4 aa, Artificial Sequence
  Synthetic
  MISC_FEATURE (4)..(4): Xaa is any amino acid
TEAX

SEQ ID NO: 68 - protein, 4 aa, Artificial Sequence
  Synthetic
  MISC_FEATURE (4)..(4): Xaa is any amino acid
TIHX

SEQ ID NO: 69 - protein, 4 aa, Artificial Sequence
  Synthetic
  MISC_FEATURE (4)..(4): Xaa is any amino acid
TVIX

SEQ ID NO: 70 - protein, 4 aa, Artificial Sequence
  Synthetic
  MISC_FEATURE (4)..(4): Xaa is any amino acid
SSSX

SEQ ID NO: 71 - protein, 4 aa, Artificial Sequence
  Synthetic
  MISC_FEATURE (4)..(4): Xaa is any amino acid
SAVX

SEQ ID NO: 72 - protein, 4 aa, Artificial Sequence
  Synthetic
  MISC_FEATURE (3)..(4): Xaa is any amino acid
SIXX

SEQ ID NO: 73 - protein, 4 aa, Artificial Sequence
  Synthetic
  MISC_FEATURE (4)..(4): Xaa is any amino acid
TQLX

SEQ ID NO: 74 - protein, 4 aa, Artificial Sequence
  Synthetic
  MISC_FEATURE (4)..(4): Xaa is any amino acid
SEIX

SEQ ID NO: 75 - protein, 4 aa, Artificial Sequence
  Synthetic
  MISC_FEATURE (4)..(4): Xaa is any amino acid
SEHX

SEQ ID NO: 76 - protein, 4 aa, Artificial Sequence
  Synthetic
  MISC_FEATURE (4)..(4): Xaa is any amino acid
SETX

SEQ ID NO: 77 - protein, 4 aa, Artificial Sequence
  Synthetic
  MISC_FEATURE (4)..(4): Xaa is any amino acid
THTX

SEQ ID NO: 78 - protein, 4 aa, Artificial Sequence
  Synthetic
  MISC_FEATURE (1)..(1): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (2)..(2): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (3)..(4): Xaa is any amino acid
XXXX

SEQ ID NO: 79 - protein, 4 aa, Artificial Sequence
  Synthetic
  MISC_FEATURE (1)..(1): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (2)..(2): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (3)..(3): Xaa is Ala, Asp, Glu, Phe, Gly, His, Ile, Lys, Leu, Met, Asn, Pro, Gln, Arg, Ser, Thr, Val, Trp, Tyr or Cys
  MISC_FEATURE (4)..(4): Xaa is any amino acid
XXXX

SEQ ID NO: 80 - protein, 125 aa, Artificial Sequence
  Synthetic
QVQLVESGGGVVQPGRSLRLSCAASGFTFSTYAMNWVRQAPGKGLEWVARIRSKYNNYATYYADSVKDRFTISRDDSKNTLYLQMNSLRAEDTAVYYCARHGNFGNSYVSWFAYWGQGTLVTVSS

SEQ ID NO: 81 - protein, 109 aa, Artificial Sequence
  Synthetic
QTVVTQEPSLTVSPGGTVTLTCRSSTGAVTTSNYANWVQQKPGQAPRGLIGGTNKRAPGVPARFSGSLLGGKAALTLSGVQPEDEAEYYCALWYSNLWVFGGGTKVEIK

SEQ ID NO: 82 - protein, 125 aa, Artificial Sequence
  Synthetic
QVQLVESGGGVVQPGRSLRLSCAASGFTFSTYAMNWVRQAPGKGLEWVARIRSKYNNYATYYADSVKDRFTISRDDSKNTLYLQMNSLRAEDTAVYYCARHGNFGNSYVSWAAYWGQGTLVTVSS

SEQ ID NO: 83 - protein, 109 aa, Artificial Sequence
  Synthetic
QTVVTQEPSLTVSPGGTVTLTCRSSTGAVTTSNYANWFQQKPGQAPRGLIGGTNKRAPGVPARFSGSLLGGKAALTLSGVQPEDEAEYYCALWYSNLWVFGGGTKVEIK

SEQ ID NO: 84 - protein, 118 aa, Artificial Sequence
  Synthetic
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSS

SEQ ID NO: 85 - protein, 107 aa, Artificial Sequence
  Synthetic
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 86 - protein, 120 aa, Artificial Sequence
  Synthetic
QVQLQQSGAELARPGASVKLSCKASGYTFTSYWMQWVKQRPGQGLEWIGTIYPGDGDTRYTQKFKGKATLTADKSSSTAYMQLSSLASEDSAVYYCARRGIPRLWYFDVWGAGTTVTVSS

SEQ ID NO: 87 - protein, 107 aa, Artificial Sequence
  Synthetic
DIQMTQTTSSLSASLGDRVTISCRASQDISNYLNWYQQKPDGTVKLLIYYTSRLHSGVPSRFSGSGSGTDYSLTIDNLEQEDIATYFCQQGNTLPPTFGGGTKLEIK

SEQ ID NO: 88 - protein, 122 aa, Artificial Sequence [sequence omitted]

SEQ ID NO: 89 - protein, 107 aa, Artificial Sequence [sequence omitted]

SEQ ID NO: 90 - protein, 120 aa, Artificial Sequence
  Synthetic
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 91 - protein, 109 aa, Artificial Sequence
  Synthetic
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 92 - protein, 120 aa, Artificial Sequence
  Synthetic
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSS

SEQ ID NO: 93 - protein, 107 aa, Artificial Sequence
  Synthetic
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 94 - protein, 115 aa, Artificial Sequence
  Synthetic
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 95 - protein, 109 aa, Artificial Sequence
  Synthetic
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 96 - protein, 113 aa, Artificial Sequence
  Synthetic
EVQLVESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 97 - protein, 107 aa, Artificial Sequence
  Synthetic
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIK

SEQ ID NO: 98 - protein, 119 aa, Artificial Sequence
  Synthetic
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 99 - protein, 112 aa, Artificial Sequence
  Synthetic
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 100 - protein, 121 aa, Artificial Sequence
  Synthetic
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 101 - protein, 112 aa, Artificial Sequence
  Synthetic
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 102 - protein, 121 aa, Artificial Sequence
  Synthetic
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 103 - protein, 108 aa, Artificial Sequence
  Synthetic
SYVLTQPPSVSVAPGQTARITCGGNNIGSKSVHWYQQPPGQAPVVVVYDDSDRPSGIPERFSGNSNGNTATLTISRVEAGDEAVYYCQVWDSSSDHVVFGGGTKLTVL

SEQ ID NO: 104 - protein, 118 aa, Artificial Sequence
  Synthetic
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 105 - protein, 108 aa, Artificial Sequence
  Synthetic
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 106 - protein, 119 aa, Artificial Sequence
  Synthetic
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSS

SEQ ID NO: 107 - protein, 113 aa, Artificial Sequence
  Synthetic
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIK

SEQ ID NO: 108 - protein, 123 aa, Artificial Sequence
  Synthetic
QVQLQESGPGLVKPSQTLSLTCTVSGGSIESGLYYWGWIRQPPGKGLEWIGSIYYSGSTYYNPSLKSRATISVDTSKNQFSLKLSSVTAADTAVYYCARDGVLALNKRSFDIWGQGTMVTVSS

SEQ ID NO: 109 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQHTVRPPLTFGGGTKVEIK

SEQ ID NO: 110 - protein, 120 aa, Artificial Sequence
  Synthetic
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 111 - protein, 107 aa, Artificial Sequence
  Synthetic
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 112 - protein, 119 aa, Artificial Sequence
  Synthetic
EVQLLESGAEVKKPGASVKVSCKASGYTFTSYYMHWVRQAPGQGLEWMGIINPSAGSTSYAQKFQGRVTMTRDTSTSTVYMELSSLRSEDTAVYYCARELMATGGFDYWGQGTLVTVSS

SEQ ID NO: 113 - protein, 110 aa, Artificial Sequence
  Synthetic
QSVLTQPASASGSPGQSITISCTGTSSDVGGYNYVSWYQQHPGKAPKLMIYDVSNRPSGVSNRFSGSKSGNTASLTISGLQAEDEANYYCSSYTSSSTNVFGTGTKVTVL

SEQ ID NO: 114 - protein, 113 aa, Artificial Sequence [sequence omitted]

SEQ ID NO: 115 - protein, 107 aa, Artificial Sequence [sequence omitted]

SEQ ID NO: 116 - protein, 120 aa, Artificial Sequence [sequence omitted]

SEQ ID NO: 117 - protein, 111 aa, Artificial Sequence [sequence omitted]

SEQ ID NO: 118 - protein, 118 aa, Artificial Sequence [sequence omitted]

SEQ ID NO: 119 - protein, 107 aa, Artificial Sequence [sequence omitted]

SEQ ID NO: 120 - protein, 123 aa, Artificial Sequence
  Synthetic
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 121 - protein, 106 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 122 - protein, 117 aa, Artificial Sequence
  Synthetic
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 123 - protein, 107 aa, Artificial Sequence
  Synthetic
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 124 - protein, 119 aa, Artificial Sequence
  Synthetic
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 125 - protein, 107 aa, Artificial Sequence
  Synthetic
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 126 - protein, 121 aa, Artificial Sequence
  Synthetic
QVQLVQSGSELKKPGASVKVSCKASGYTFTVFGMNWVRQAPGQGLEWMGWINTKTGEATYVEEFKGRFVFSLDTSVSTAYLQISSLKADDTAVYYCARWDFYDYVEAMDYWGQGTTVTVSS

SEQ ID NO: 127 - protein, 108 aa, Artificial Sequence
  Synthetic
DIQMTQSPSSLSASVGDRVTITCKASQNVGTNVAWYQQKPGKAPKLLIYSASYRYSGVPSRFSGSGSGTDFTFTISSLQPEDIATYYCHQYYTYPLFTFGQGTKVEIK

SEQ ID NO: 128 - protein, 123 aa, Artificial Sequence [sequence omitted]

SEQ ID NO: 129 - protein, 107 aa, Artificial Sequence [sequence omitted]

SEQ ID NO: 130 - protein, 121 aa, Artificial Sequence
  Synthetic
EVQLVESGGGLVQPGGSLRLSCAASGFSINGSWIFWVRQAPGKGLEWVGAIWPFGGYTHYADSVKGRFTISADTSKNTAYLQMNSLRAEDTAVYYCARWGHSTSPWAMDYWGQGTLVTVSS

SEQ ID NO: 131 - protein, 107 aa, Artificial Sequence
  Synthetic
DIQMTQSPSSLSASVGDRVTITCRASQVIRRSLAWYQQKPGKAPKLLIYAASNLASGVPSRFSGSGSGTDFTLTISSLQPEDFATYYCQQSNTSPLTFGQGTKVEIK

SEQ ID NO: 132 - protein, 120 aa, Artificial Sequence
  Synthetic
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSS

SEQ ID NO: 133 - protein, 107 aa, Artificial Sequence
  Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIK

SEQ ID NO: 134 - protein, 120 aa, Artificial Sequence
  Synthetic
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 135 - protein, 108 aa, Artificial Sequence
  Synthetic
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 136 - protein, 120 aa, Artificial Sequence
  Synthetic
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSS

SEQ ID NO: 137 - protein, 108 aa, Artificial Sequence; identical to SEQ ID NO: 135
  Synthetic

SEQ ID NO: 138 - protein, 117 aa, Artificial Sequence
  Synthetic
QVQLKQSGPGLLQPSQSLSISCTVSGFSLATYGVHWVRQSPGKGLEWLGVIWRGGSTDYSAAFMSRLSITKDNSKSQVFFKMNSLQADDTAIYFCAKQAYGHYMDYWGQGTSVTVSS

SEQ ID NO: 139 - protein, 112 aa, Artificial Sequence
  Synthetic
DVLMTQTPLSLPVSLGDQASISCRSSQNIVHSNGNTYLEWYLQKPGQSPKLLIYKVSNRFSGVPDRFSGSGSGTDFTLKITRLEAEDLGVYYCFQGSHVPWTFGGGTKLEIK

SEQ ID NO: 140 - protein, 117 aa, Artificial Sequence
  Synthetic
EVQLVESGGGLVQPGGSLRLSCAASGFSFATYGVHWVRQSPGKGLEWLGVIWRGGSTDYSAAFMSRLTISKDNSKNTVYLQMNSLRAEDTAVYFCAKQAYGHYMDYWGQGTSVTVSS

SEQ ID NO: 141 - protein, 112 aa, Artificial Sequence
  Synthetic
DVVMTQSPLSLPVTLGQPASISCRSSQNIVHSNGNTYLEWYLQRPGQSPRLLIYKVSNRFSGVPDRFSGSGSGTDFTLKISRVEAEDVGVYYCFQGSHVPWTFGQGTKVEIK

SEQ ID NO: 142 - protein, 119 aa, Artificial Sequence
  Synthetic
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 143 - protein, 112 aa, Artificial Sequence
  Synthetic
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 144 - protein, 117 aa, Artificial Sequence; identical to SEQ ID NO: 138
  Synthetic

SEQ ID NO: 145 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 146 - protein, 118 aa, Artificial Sequence
  Synthetic
QVQLQQSGAELAKPGASVKLSCKASGYTFTSSWMHWIKQRPGQGLEWLGYINPSSGYTKYNRKFRDKATLTADKSSSTAYMQLTSLTYEDSAVYYCARGGNGYYFDYWGQGTTLTVSS

SEQ ID NO: 147 - protein, 106 aa, Artificial Sequence
  Synthetic
DIVMTQSHKFMSTSVGDRVSITCKASQDVGTAVAWYQQKPGQSPKLLIYWASTRHTGVPDRFTGSRYGTDFTLTISNVQSEDLADYLCQQYSNYITFGGGTKLELK

SEQ ID NO: 148 - protein, 118 aa, Artificial Sequence
  Synthetic
QVQLQQSGAELAKPGASVKLSCKASGYTFTTYWMHWVKQRPGQGLEWIGYINPSSGYTKYNQKFKVKATLTADKSSSTAYMQLSSLTDEDSAVYYCANGNLGYFFDYWGQGTTLTVSS

SEQ ID NO: 149 - protein, 106 aa, Artificial Sequence
  Synthetic
DIVMTQSHKFMSTSVGDRVSITCKASQDVGTAVAWYQQKPGLSPKLLIYWASTRHTGVPDRFTGSGSGTDFTLTISNVQSEDLADYFCQQYSSYLTFGAGTKLEIK

SEQ ID NO: 150 - protein, 118 aa, Artificial Sequence
  Synthetic
EVQLQQSGAELVKPGASVKLSCTASGFNIKDYYMHWIKQRPGQGLEWLGYINPSSGYTKYNRKFRDKATLTADKSSSTAYMQLTSLTYEDSAVYYCARGGNGYYLDYWGQGTTLTVSS

SEQ ID NO: 151 - protein, 106 aa, Artificial Sequence
  Synthetic
DIQMTQSHKFMSTSVGDRVSITCKASQDVSTAVAWYQQKPGQSPKLLIYWASTRHTGVPDRFTGSGSGTDFTLTISNVQSEDLADYLCQQYSNYITFGAGTKLELK

SEQ ID NO: 152 - protein, 115 aa, Artificial Sequence
  Synthetic
EVQLQQSGPGLVKPTQSLSLTCSVTGYSITSGYYWTWIRQFPGNNLEWIGYISFDGTNDYNPSLKNRISITRDTSENQFFLKLNSVTTEDTATYYCARGPPCTYWGQGTLVTVSA

SEQ ID NO: 153 - protein, 106 aa, Artificial Sequence
  Synthetic
DIVLTQSPAIMSASLGEKVTMSCRATSSVNYIYWYQQKSDASPKLWIFYTSNLAPGVPPRFSGSGSGNSYSLTISSMEAEDAATYYCQQFSSSPWTFGGGTKLEIK

SEQ ID NO: 154 - protein, 122 aa, Artificial Sequence
  Synthetic
QVQLQQSGPELVKPGASVKMSCKASGYTFTHFVLHWVKQNPGQGLEWIGYIIPYNDGTKYNEKFKGKATLTSDKSSSTAYMELSSLTSEDSAVYYCARGNRYDVGSYAMDYWGQGTSVTVSS

SEQ ID NO: 155 - protein, 113 aa, Artificial Sequence
  Synthetic
DIVMTQSPSSLAVSVGEKVTMSCKSSQSLLYSSNQKNYLAWYQQKPGQSPKLLIYWASTRESGVPDRFTGSGSGTDFTLTISSVKAEDLAVYLCQQYYRFPYTFGGGTKLEIK

SEQ ID NO: 156 - protein, 119 aa, Artificial Sequence
  Synthetic
QVQLQQSGPELVKPGASVKISCKASGYTFTDNNMDWVKQSHGKGLEWIGDINTKSGGSIYNQKFKGKATLTIDKSSSTAYMELRSLTSEDTAVYYCARRRSYGYYFDYWGQGTTLTVSS

SEQ ID NO: 157 - protein, 107 aa, Artificial Sequence
  Synthetic
DIVLTQSQKFMSTSVGDRVSITCKASQNVGTAVAWYQQKPGQSPKALIYSASYRYSGVPDRFTGSGSGTDFTLTISNVQSEDLAEYFCQQYNSYPLTFGAGTKLEIK

SEQ ID NO: 158 - protein, 120 aa, Artificial Sequence [sequence omitted]

SEQ ID NO: 159 - protein, 107 aa, Artificial Sequence [sequence omitted]

SEQ ID NO: 160 - protein, 119 aa, Artificial Sequence [sequence omitted]

SEQ ID NO: 161 - protein, 107 aa, Artificial Sequence [sequence omitted]

SEQ ID NO: 162 - protein, 121 aa, Artificial Sequence
  Synthetic
EVQILETGGGLVKPGGSLRLSCATSGFNFNDYFMNWVRQAPEKGLEWVAQIRNKIYTYATFYAESLEGRVTISRDDSESSVYLQVSSLRAEDTAIYYCTRSLTGGDYFDYWGQGVMVTVSS

SEQ ID NO: 163 - protein, 110 aa, Artificial Sequence
  Synthetic
DIVLTQSPALAVSLGQRATISCRASQSVTISGYSFIHWYQQKPGQQPRLLIYRASNLASGIPARFSGSGSGTDFTLTINPVQADDIATYFCQQSRKSPWTFAGGTKLELR

SEQ ID NO: 164 - protein, 121 aa, Artificial Sequence
  Synthetic
EVQLVESGGGLVQPGGSLRLSCAASGFNFNDYFMNWVRQAPGKGLEWVAQIRNKIYTYATFYAASVKGRFTISRDNAKNSLYLQMNSLRAEDTAVYYCARSLTGGDYFDYWGQGTLVTVSS

SEQ ID NO: 165 - protein, 111 aa, Artificial Sequence
  Synthetic
EILMTQSPATLSLSPGERATLSCRASQSVTISGYSFIHWYQQKPGQAPRLLIYRASNLASGIPARFSGSGSGTDFTLTISSLEPEDFALYYCQQSRKSPWTFGQGTKVEIK

SEQ ID NO: 166 - protein, 118 aa, Artificial Sequence
  Synthetic
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 167 - protein, 112 aa, Artificial Sequence
  Synthetic
DVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIK

SEQ ID NO: 168 - protein, 5 aa, Artificial Sequence
  Synthetic
DKTHT

SEQ ID NO: 169 - protein, 6 aa, Artificial Sequence
  Synthetic
EKCCVE

SEQ ID NO: 170 - protein, 54 aa, Artificial Sequence
  Synthetic
GDTTHTCPRCPEPKSCDTPPPCPRCPEPKSCDTPPPCPRCPEPKSCDTPPPCPR

SEQ ID NO: 171 - protein, 4 aa, Artificial Sequence
  Synthetic
YGPP

SEQ ID NO: 172 - protein, 107 aa, Artificial Sequence [sequence omitted]

SEQ ID NO: 173 - protein, 106 aa, Artificial Sequence
  Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 174 - protein, 106 aa, Artificial Sequence
  Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 175 - protein, 106 aa, Artificial Sequence
  Synthetic
GQPKAAPSVTLFPPSSEELQANKATLVCLISDFYPGAVTVAWKADSSPAKAGVETTTPSKQSNNKYAASSYLSLTPEQWKSHKSYSCQVTHEGSTVEKTVAPTECS

SEQ ID NO: 176 - protein, 106 aa, Artificial Sequence
  Synthetic
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 177 - protein, 106 aa, Artificial Sequence
  Synthetic
GQPKAAPTVTLFPPSSEELQANKATLVCLISDFYPGAVKVAWKADSSPAKAGVETTTPSKQSNNKYAASSYLSLTPEQWKSHKSYSCQVTHEGSTVEKTVAPTECS

SEQ ID NO: 178 - protein, 105 aa, Artificial Sequence [sequence omitted]

SEQ ID NO: 179 - protein, 103 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 180 - protein, 94 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKV

SEQ ID NO: 181 - protein, 99 aa, Artificial Sequence
  Synthetic
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVE

SEQ ID NO: 182 - protein, 97 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKR

SEQ ID NO: 183 - protein, 115 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 184 - protein, 114 aa, Artificial Sequence
  Synthetic
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 185 - protein, 114 aa, Artificial Sequence
  Synthetic
CPPCPAPPVAGPSVFLFPPKPKDTLMISRTPEVTCVVVDVSHEAPEVQFNWYVDGVEVHNAKTKPREEQFNSTFRVVSVLTVVHQDWLNGKEYKCKVSNKGLPAPIEKTISKTK

SEQ ID NO: 186 - protein, 112 aa, Artificial Sequence
  Synthetic
CPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 187 - protein, 115 aa, Artificial Sequence
  Synthetic
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 188 - protein, 107 aa, Artificial Sequence
  Synthetic
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 107 aa, Artificial Sequence
  Synthetic
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 190 - protein, 107 aa, Artificial Sequence
  Synthetic
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 191 - protein, 107 aa, Artificial Sequence
  Synthetic
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 192 - protein, 107 aa, Artificial Sequence
  Synthetic
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 107 aa, Artificial Sequence
  Synthetic
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 194 - protein, 107 aa, Artificial Sequence
  Synthetic
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 107 aa, Artificial Sequence
  Synthetic
GQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 196 - protein, 107 aa, Artificial Sequence
  Synthetic
GQPREPQVYTLPPSRDELTKNQVSLTCLVKGFYPSDIAVEWESNGQPENNYKTTPPVLDSDGSFFLVSKLTVDKSRWQQGNVFSCSVMHEALHNHYTQKSLSLSPGK

SEQ ID NO: 197 - protein, 107 aa, Artificial Sequence
  Synthetic
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 198 - protein, 180 aa, Artificial Sequence
  Synthetic
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTF

SEQ ID NO: 199 - protein, 75 aa, Artificial Sequence
  Synthetic
EVHNLQPEKVQTLEAWVIHGGREDSRDLCQDPTIKELESIISKRNIQFSCKNIYRPDKFLQCVKNPEDSSCTSEI

SEQ ID NO: 200 - protein, 174 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIED

SEQ ID NO: 201 - protein, 63 aa, Artificial Sequence
  Synthetic
VQLADHYQQNTPIGDGPVLLPDNHYLSTQSALSKDPNEKRDHMVLLEFVTAAGITLGMDELYK

SEQ ID NO: 202 - protein, 4 aa, Artificial Sequence
  Synthetic
  MISC_FEATURE (4)..(4): Xaa is any amino acid
DKGX

SEQ ID NO: 203 - protein, 4 aa, Artificial Sequence
  Synthetic
  MISC_FEATURE (4)..(4): Xaa is any amino acid
DKTX

SEQ ID NO: 204 - protein, 4 aa, Artificial Sequence
  Synthetic
  MISC_FEATURE (3)..(4): Xaa is any amino acid
STXX

SEQ ID NO: 205 - protein, 214 aa, Artificial Sequence
  Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 448 aa, Artificial Sequence
  Synthetic
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSICPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 207 - protein, 452 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 214 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0332757 A1
(43) Pub. Date: Oct. 20, 2022

(54) SPLIT INTEIN AND PREPARATION METHOD FOR RECOMBINANT POLYPEPTIDE USING THE SAME

(71) Applicant: Wuhan YZY Biopharma Co., Ltd., Wuhan (CN)

(72) Inventors: Jing Zhang, Wuhan (CN); Fang Luo, Wuhan (CN); Cheng Gong, Wuhan (CN); Xin Wang, Wuhan (CN); Lijuan Wang, Wuhan (CN); Pengfei Zhou, Wuhan (CN)

(21) Appl. No.: 17/641,431

(22) PCT Filed: Sep. 9, 2020
(86) PCT No.: PCT/CN2020/114271

§ 371 Date: Mar. 8, 2022

(30) Foreign Application Priority Data
Sep. 9, 2019 (CN) 201910850928.1

(51) Int. Cl. C07K 5/06; C12N 15/62

(52) U.S. Cl. CPC C07K 5/06; C12N 15/62; A61K 2039/505

(57) ABSTRACT

The present disclosure relates to a pair of flanking sequences for a split intein, wherein the pair of flanking sequences includes: a flanking sequence a and a flanking sequence b; the flanking sequence a is located at the N-terminus of the split intein N-terminal protein splicing region (In), and is between the N-terminal extein (En) and the In; the flanking sequence b is located at the C-terminus of the split intein C-terminal protein splicing region (Ic), and is between the Ic and the C-terminal extein (Ec); and the split intein is selected from the group consisting of SspDnaE, SspDnaB, MxeGyrA, MjaTFIIB, PhoVMA, TVoVMA, Gp41-1, Gp41-8, IMPDH-1 and PhoRadA.

### FIELD OF THE INVENTION

The present disclosure relates to split inteins containing novel flanking sequence pairs, and recombinant polypeptides using the same, and the use of the inteins in the preparation of antibodies, in particular bispecific antibodies. The present disclosure also relates to a method of screening for the split inteins containing novel flanking sequence pairs.

### BACKGROUND OF THE INVENTION

Protein trans-splicing refers to a protein splicing reaction mediated by split inteins. During the splicing process, firstly the N-terminal fragment or N-terminal protein splicing region (In) and the C-terminal fragment or C-terminal protein splicing region (Ic) of the split intein recognize each other and are non-covalently bound; once the structure is correctly folded after binding, the split intein with a reconstructed active center completes the protein splicing reaction according to the typical protein splicing pathway, and connects the exteins at both sides (Saleh. L., Chemical Record. 6 (2006) 183-193).

In the technology of preparing recombinant proteins, a gene expressing a precursor protein can be split into two open reading frames, and a split intein consisting of two parts, N′fragment of intein (referred to as In) and C′fragment of intein (referred to as Ic), is used to catalyze the protein trans-splicing reaction, so that the two split exteins (En, Ec) that constitute the precursor protein are linked by a peptide bond, thereby obtaining a recombinant protein (Ozawa. T., Nat Biotechbol. 21 (2003) 287-93).

A bispecific antibody refers to an antibody molecule that can recognize two antigens or two epitopes, such as a bispecific or multispecific antibody capable of binding two or more antigens, which is known in the art and can be obtained in a eukaryotic expression system or in a prokaryotic expression system by a cell fusion method, chemical modification method, gene recombination method and other methods.

Currently, a wide variety of recombinant bispecific antibody formats have been developed, for example, a tetravalent bispecific antibody by fusing e.g. a IgG antibody format with a single chain domain (see e.g. Coloma, M J, et al., Nature Biotech. 15 (1997) 159-163; WO 2001077342; and Morrison, S., L., Nature Biotech. 25(2007) 1233-1234). However, due to the large difference from natural antibodies in structure, such antibodies will cause a strong immune response and a short half-life in vivo.

In addition, several other novel formats capable of binding two or more antigens have also been developed, e.g., small molecule antibodies such as minibodies, several single chain formats (scFv, bi-scFv), and the like. In these small molecule antibodies, the antibody core structure (IgA, IgD, IgE, IgG or IgM) is no longer maintained (Holliger, P., et al., Nature Biotech. 23 (2005) 1126-1136; Fischer, N, and Leger, O., Pathobiology 74 (2007) 3-14; Shen, J., et al., J. Immunol. Methods. 318 (2007) 65-74; Wu, C., et al., Nature Biotech. 25 (2007) 1290-1297).

There are obvious advantages over bispecific antibodies by linking a core binding region of an antibody to a core binding region of other antibodies via a linker, however, there are also some problems in its application as a medicament, which greatly limits its use in preparation of medicine.

In fact, in terms of immunogenicity, these foreign proteins may elicit an immune response against the linker per se, or against the linker-containing protein, or even cause an immune storm. In addition, due to the flexibility, these linkers are prone to protein degradation, which can easily lead to poor stability, easy aggregation, shortened half-life of the antibody and may further enhance immunogenicity. For example, Blinatumomab of Amgen has a half-life of only 1.25 hours in blood, resulting in a 24-hour continuous administration via a syringe pump, which greatly limits its application (Bargou, R and Leo. E., Science. 321 (2008) 974-7).

In addition, it is desirable that in the engineering of bispecific antibodies, effector functions of antibody Fc fragment are retained, for example, CDC (complement-dependent cytotoxicity), or ADCC (cytotoxicity), and prolonged half-life of antibody binding to FcRn (Fc receptor) at blood vessel endothelium. These functions must be mediated by the Fc region, therefore, the Fc region should be retained in the engineered bispecific antibody.

Therefore, there is a need to develop bispecific antibodies that are structurally very similar to those of naturally occurring antibodies (e.g., IgA, IgD, IgE, IgG, IgM), and furthermore, humanized bispecific antibodies with minimal sequence differences from human antibodies and complete human bispecific antibodies are required.

At present, attempts have been made to prepare bispecific antibodies by the trans-splicing mechanism of Npu-PCC73102 DnaE (abbreviated as NpuDnaE) intein. There is not a linker peptide in the obtained spliced product by preparing a bispecific antibody via the intein trans-splicing mechanism, however, there still exist the following problems: in the bispecific antibody thus obtained, a free sulfhydryl group introduced by the Ic flanking sequence cannot be avoided, leading to a great risk of misfolding and instability, as well as undesirable splicing efficiency (Han L, Zong H, et al., Naturally split intein Npu DnaE mediated rapid generation of bispecific IgG antibodies, Methods., Vol 154, 2019 Feb 1;154:32-37).

The efficiency of split intein-mediated protein splicing is directly related to the intein sequence and flanking sequences of the intein.

In the NEB database (http://inteins.com/), more than 600 split inteins are listed, wherein the commonly used ones are for example NpuDnaE and SspDnaE. However, based on the flanking sequences of these inteins, for example, the In flanking sequence of NpuDnaE being AEY (En-AEY-In), the Ic flanking sequence of NpuDnaE being CFNGT (Ic-CFNGT-Ec), the In flanking sequence of SspDnaE being AEY (En-AEY-In) and the Ic flanking sequence of SspDnaE being CFNKS (Ic-CFNKS-Ec), it can be seen that the protein format of En-AEY-In and Ic-CFNGT-Ec after splicing is En-AEYCFNGT-Ec, and the protein format of En-AEY-In and Ic-CFNKS-Ec after splicing is En-AEYCFNKS-Ec, both of which have a cysteine residue. Therefore, there is a free sulfhydryl group in the spliced product, which greatly increases the risk of misfolding and instability of the product.

In order to avoid the free sulfhydryl group in the spliced product, the existing flanking sequences pairs of split inteins need to be improved, and novel flanking sequences that maintain the good splicing efficiency of intein and do not contain a cystine residue are needed.

It has been reported that some split inteins have serine or threonine instead of cysteine in their Ic flanking sequences, for example, SspDnaB, TVoVMA, MxeGyrA, PhoRadA, Gp41-1, Gp41-8, Nrdj-1, IMPDH-1, etc. (Bareket Dassa, et al. Nucleic Acids Res. 2009 May; 37(8): 2560-2573). These inteins can be used to prevent generation of free sulfydryl groups at the junction of the spliced product. However, there is no report on the preparation of bispecific antibodies by using these inteins.

In addition, amino acid mutations in the flanking sequence pairs of existing split inteins will affect the efficiency of trans-splicing. Therefore, a screening method is needed to screen an intein containing a novel flanking sequence pair with excellent trans-splicing efficiency and without introducing free sulfhydryl groups at the junction into the spliced product. Furthermore, there is a need for a split intein suitable for the preparation of antibodies, especially bispecific antibodies, which has excellent trans-splicing efficiency and does not introduce free sulfhydryl groups at the junction in the spliced product and contains novel flanking sequence pairs.

### SUMMARY OF THE INVENTION

In the present disclosure, through performing regular amino acid mutations on the flanking sequences pairs of existing intein and screening for the flanking sequence pairs with excellent trans-splicing efficiency, a split intein with novel flanking sequence pairs is obtained, which has flanking sequences without cysteine residues, does not introduce free sulfhydryl groups at the junction in the spliced product, has an excellent trans-splicing efficiency, and is especially suitable for the preparation of antibodies (especially bispecific antibodies).

By using the split intein of the present disclosure, under relatively mild conditions (such as normal temperature, physiological salt concentration, neutral pH, etc.), polypeptide fragments from different proteins can be spliced together with high splicing efficiency to form a recombinant fusion polypeptide protein.

In addition, based on the screening of the above split inteins, the inventors established a method for preparing recombinant polypeptides (especially bispecific antibodies) by using the split inteins. The bispecific antibody thus prepared does not contain a non-natural domain, has a structure closely similar to that of natural antibody (IgA, IgD, IgE, IgG or IgM), and has a Fc domain. The bispecific antibody has a complete structure and good stability, and can retain or remove CDC (complement-dependent cytotoxicity) or ADCC (antibody-dependent cytotoxicity) or ADCP (antibody-dependent cellular phagocytosis) or FcRn (Fc receptor)-binding activity according to different IgG subclasses.

The bispecific antibody prepared by the method of the present disclosure has the following advantages: the bispecific antibody has a long half-life in vivo and low immunogenicity, and does not introduce any form of linkers; has an improved stability, and reduced in vivo immune response.

The bispecific antibody prepared by the method of the present disclosure can be prepared by a mammalian cell expression system, so that it has the same glycosylation modification as that of wild-type IgG, has better biological function, is more stable, and has a long half-life in vivo; the in vitro splicing method by using inteins can completely avoid the problems of heavy chain mismatch and light chain mismatch commonly found in traditional methods.

The preparation method for bispecific antibodies of the present disclosure can also be used to produce humanized bispecific antibodies and bispecific antibodies with complete human sequences. The sequence of such an antibody prepared by the method of the present disclosure is more similar to that of a human antibody, which can effectively reduce the immune response.

The preparation method for bispecific antibodies of the present disclosure is a method for constructing universal bispecific antibodies, which is not limited by antibody subclasses (IgG, IgA, IgM, IgD, IgE, and light chain κ and λ types), and does not need to design different mutations according to a specific target and can be used to construct any bispecific antibody.

The present disclosure provides the following technical solutions.

1. A flanking sequence pair for a split intein, wherein,

the flanking sequence pair comprises: a flanking sequence a and a flanking sequence b; wherein, the flanking sequence a is located at N-terminus of a split intein N-terminal protein splicing region (In), and is between a N-terminal extein (En) and the In; the flanking sequence b is located at C-terminus of a split intein C-terminal protein splicing region (Ic), and is between the Ic and a C-terminal extein (Ec);

the split intein is selected from the group consisting of SspDnaE, SspDnaB, MxeGyrA, MjaTFIIB, PhoVMA, TVoVMA, Gp41-1, Gp41-8, IMPDH-1 or PhoRadA,

(1) when the split intein is IMPDH-1,

the flanking sequence a is A₋₃A₋₂A₋₁ and the flanking sequence b is B₁B₂B₃, wherein:

A₋₃ is X or deletion, or preferably G or D; A₋₂, is X or deletion, or preferably G or K; A₋₁ is selected from G or T;

B₁ is S; B₂ is I or T or S; B₃ is X or deletion;

preferably,

the flanking sequence a is G, XG, XGG, DKG or DKT, and the flanking sequence b is SI, ST, SS, SIX, STX or SSX;

(2) when the split intein is Gp41-8,

the flanking sequence a is A₋₃A₋₂A₋₁ and the flanking sequence b is B₁B₂B₃, wherein:

A₋₃ is X or deletion; A₋₂ is selected from N or D; A₋₁ is selected from R or K;

B₁ is S or T; B₂ is A or H; B₃ is X or deletion, or preferably V, Y or T,

preferably,

the flanking sequence a is NR, XNR, DK, XDK, DR or XDR, and the flanking sequence b is SA or SAX;

(3) when the split intein is SspDnaB,

the flanking sequence a is A₋₃A₋₂A₋₁ and the flanking sequence b is B₁B₂B₃, wherein:

A₋₃ is X or deletion; A₋₂ is selected from S or D; A₋₁ is selected from G or K;

B₁ is S; B₂ is I; B₃ is X or deletion, or preferably E or T,

preferably,

the flanking sequence a is SG, XSG, DK, XDK, and the flanking sequence b is SI or SIX;

(4) when the intein is MjaTFIIB,

the flanking sequence a is A₋₃A₋₂A₋₁, and the flanking sequence b is B₁B₂B₃, wherein

A₋₃ is X or deletion; A₋₂ is selected from T or D; A₋₁ is selected from Y;

B₁ is T; B₂ is I or H; B₃ is X or deletion, or preferably H or T;

preferably,

the flanking sequence a is TY, DY, XTY or XDY, and the flanking sequence b is TI, TIX, TH or THX;

(5) when the split intein is PhoRadA,

the flanking sequence a is A₋₃A₋₂A₋₁ and the flanking sequence b is B₁B₂B₃, wherein:

A₋₃ is X or deletion; A₋₂ is selected from G or D; A₋₁ is selected from K;

B₁ is T; B₂ is Q or H; B₃ is X or deletion, or preferably L or T,

preferably,

the flanking sequence a is GK, XGK, DK or XDK, and the flanking sequence b is TQ, TH, TQX or THX;

(6) when the split intein is TVoVMA,

the flanking sequence a is A₋₃A₋₂A₋₁ and the flanking sequence b is B₁B₂B₃, wherein:

A₋₃is X or deletion; A₋₂ is selected from G or D; A₋₁ is K;

B₁ is T; B₂ is V or H; B₃ is X or deletion, or preferably I or T,

preferably,

the flanking sequence a is GK, XGK, DK or XDK, and the flanking sequence b is TV, TH, TVX or THX;

(7) when the split intein is MxeGyrA,

the flanking sequence a is A₋₃A₋₂A₋₁ and the flanking sequence b is B₁B₂B₃, wherein:

A₋₃ is X or deletion; A₋₂ is selected from R or D; A₋₁ is selected from Y, K or T;

B₁ is T; B₂ is E or H; B₃ is X or deletion, or preferably A or T,

preferably,

the flanking sequence a is RY, XRY, DK or XDK, and the flanking sequence b is TE, TH, TEX or THX;

(8) when the split intein is PhoVMA,

the flanking sequence a is A₋₃A₋₂A₋₁ and the flanking sequence b is B₁B₂B₃, wherein:

A₋₃is X or deletion; A₋₂ is selected from G or D; A₋₁ is selected from K;

B₁ is T; B₂ is V or H; B₃ is X or deletion, or preferably I or T,

preferably,

the flanking sequence a is GK, XGK, DK or XDK, and the flanking sequence b is TV, TH, TVX or THX;

(9) when the split intein is Gp41-1,

the flanking sequence a is A₋₃A₋₂A₋₁ and the flanking sequence b is B₁B₂B₃, wherein:

A₋₃ is X or deletion; A₋₂ is selected from G or D; A₋₁ is selected from Y or K;

B₁ is S or T; B₂ is S or H; B₃ is X or deletion, or preferably S or T;

preferably,

the flanking sequence a is GY, XGY, DK or XDK, and the flanking sequence b is SS, SH, SSX or SHX;

(10) when the split intein is SspDnaE,

the flanking sequence a is A₋₃A₋₂A₋₁ and the flanking sequence b is B₁B₂B₃, wherein:

A₋₃is X or deletion; A₋₂ is selected from G or D; A₋₁ is selected from G, S or K;

B₁ is T or S; B₂ is E or H; B₃ is X or deletion, or preferably T;

preferably,

the flanking sequence a is GG, XGG, GK, XGK, DK or XDK, and the flanking sequence b is SE, TH, SEX or THX;

wherein the X is any amino acid selected from the group consisting of G, A, V, L, M, I, S, T, P, N, Q, F, Y, W, K, R, H, D, E, C.

2. The flanking sequence pair for a split intein according to item 1, wherein the split intein together with the flanking sequence pair are used for trans-splicing,

wherein,

the SspDnaE is composed of the In of sequence as SEQ ID NO:31 and the Ic of sequence as SEQ ID NO:32,

the SspDnaB is composed of the In of sequence as SEQ ID NO:33 and the Ic of sequence as SEQ ID NO:34,

the MxeGyrA is composed of the In of sequence as SEQ ID NO:35 and the Ic of sequence as SEQ ID NO:36,

the MjaTFIIB is composed of the In of sequence as SEQ ID NO:37 and the Ic of sequence as SEQ ID NO:38,

the PhoVMA is composed of the In of sequence as SEQ ID NO:39 and the Ic of sequence as SEQ ID NO:40,

the TvoVMA is composed of the In of sequence as SEQ ID NO:41 and the Ic of sequence as SEQ ID NO:42,

the Gp41-1 is composed of the In of sequence as SEQ ID NO:43 and the Ic of sequence as SEQ ID NO:44,

the Gp41-8 is composed of the In of sequence as SEQ ID NO:45 and the Ic of sequence as SEQ ID NO:46,

the IMPDH-1 is composed of the In of sequence as SEQ ID NO:47 and the Ic of sequence as SEQ ID NO:48,

the PhoRadA is composed of the In of sequence as SEQ ID NO:49 and the Ic of sequence as SEQ ID NO:50,

preferably,

(1) when the split intein is IMPDH-1, the flanking sequence a is XGG and the flanking sequence b is SI, ST, SS; or the flanking sequence a is DKG and the flanking sequence b is SI, ST, SS; or the flanking sequence a is DKT and the flanking sequence b is SI, ST, SS;

(2) when the split intein is Gp41-8, the flanking sequence a is NR and the flanking sequence b is SAV; or the flanking sequence a is DK and the flanking sequence b is SAV; the flanking sequence a is NR and the flanking sequence b is SAT; or the flanking sequence a is DK and the flanking sequence b is SAT;

(3) when the split intein is SspDnaB, the flanking sequence a is SG and the flanking sequence b is SIE;

(4) when the split intein is PhoRadA, the flanking sequence a is GK and the flanking sequence b is TQL or THT; or the flanking sequence a is DK and the flanking sequence b is TQL or THT;

(5) when the split intein is TVoVMA, the flanking sequence a is GK and the flanking sequence b is TVI or THT; or the flanking sequence a is DK and the flanking sequence b is TVI or THT;

(6) when the split intein is MxeGyrA, the flanking sequence a is RY and the flanking sequence b is TEA or THT; or the flanking sequence a is DK and the flanking sequence b is TEA or THT;

(7) when the split intein is MjaTFIIB, the flanking sequence a is TY and the flanking sequence b is TIH; or the flanking sequence a is TY and the flanking sequence b is THT;

(8) when the split intein is PhoVMA, the flanking sequence a is GK and the flanking sequence b is TVI or THT; or the flanking sequence a is DK and the flanking sequence b is TVI or THT;

(9) when the split intein is Gp41-1, the flanking sequence a is GY and the flanking sequence b is SSS or SHT; or the flanking sequence a is DK and the flanking sequence b is SSS or SHT;

(10) when the split intein is SspDnaE, the flanking sequence a is GG and the flanking sequence b is SET or THT; or the flanking sequence a is GK and the flanking sequence b is SET or THT; or the flanking sequence a is DK and the flanking sequence b is SET or THT;

wherein the X is any amino acid selected from the group consisting of G, A, V, L, M, I, S, T, P, N, Q, F, Y, W, K, R, H, D, E, C.

3. A recombinant polypeptide obtained by trans-splicing via the flanking sequence pair for a split intein according to item 1 or 2.

4. The recombinant polypeptide according to item 3, wherein the recombinant polypeptide is obtained by a component A and a component B through trans-splicing;

in the component A, the N-terminus of the flanking sequence a is connected to the C-terminus of the En, and the C-terminus of the flanking sequence a is connected to the In, optionally a tag protein is connected to the C-terminus of the In;

in the component B, the C-terminus of the flanking sequence b is connected to the N-terminus of the Ec, and the N-terminus of the flanking sequence b is connected to the Ic, optionally a tag protein is connected to the N-terminus of the Ic;

wherein, coding sequences of the En and the Ec are respectively derived from a N-terminal part and a C-terminal part of the same protein,

preferably, the tag protein is selected from SEQ ID NO: 24, 25, 26, 27, 28, 29 or 30.

5. The recombinant polypeptide according to item 3, wherein the recombinant polypeptide is obtained by a component A and a component B through trans-splicing;

in the component A, the N-terminus of the flanking sequence a is connected to the C-terminus of the En, and the C-terminus of the flanking sequence a is connected to the In, optionally a tag protein is connected to the C-terminus of the In;

in the component B, the C-terminus of the flanking sequence b is connected to the N-terminus of the Ec, and the N-terminus of the flanking sequence b is connected to the Ic, optionally a tag protein is connected to the N-terminus of the Ic;

wherein, coding sequences of the En and the Ec are derived from different proteins.

6. The recombinant polypeptide according to item 4 or 5, wherein the recombinant polypeptide is a fluorescent protein, protease, signal peptide, antimicrobial peptide, antibody, or a polypeptide with biological toxicity.

7. The recombinant polypeptide according to item 4 or 5, wherein the same protein, or one or more of the different proteins is an antibody.

8. The recombinant polypeptide according to item 7, wherein the antibody is a natural immunoglobulin class IgG, IgM, IgA, IgD or IgE, or an immunoglobulin subclass: IgG1, IgG2, IgG3, IgG4, IgG5, or with light chains of different classes: kappa, lambda; or a single domain antibody; or

the antibody is a full-length antibody or a functional fragment of an antibody.

9. The recombinant polypeptide according to item 8, wherein the functional fragment of an antibody is selected from one or more of the group consisting of: antibody heavy chain variable region VH, antibody light chain variable region VL, antibody heavy chain constant region fragment Fc, antibody heavy chain constant region 1 CH1, antibody heavy chain constant region 2 CH2, antibody heavy chain constant region 3 CH3, antibody light chain constant region CL or single domain antibody variable region VHH.

10. The recombinant polypeptide according to item 7, wherein, the same protein or one or more of the different proteins is specific to an antigen or epitope A,

the antigen A comprises: tumor cell surface antigen, immune cell surface antigen, cytokine, cytokine receptor, transcription factor, membrane protein, actin, virus, bacteria, endotoxin, FIXa, FX, CD3, SLAMF7, CD38, BCMA, CD20, CD16, CEA, PD-L1, PD-1, CTLA-4, TIGIT, LAG-3, VEGF, B7-H3, Claudin18.2, TGF-β, Her2, IL-10, Siglec-15, Ras, C-myc, and the epitope A is an immunogenic epitope of the antigen A.

11. The recombinant polypeptide according to item 10, wherein, the same protein or one or more of the different proteins is specific to an antigen or epitope B different from the antigen or epitope A,

the antigen B comprises: tumor cell surface antigen, immune cell surface antigen, cytokine, cytokine receptor, transcription factor, membrane protein, actin, virus, bacteria, endotoxin, FIXa, FX, CD3, SLAMF7, CD38, BCMA, CD20, CD16, CEA, PD-L1, PD-1, CTLA-4, TIGIT, LAG-3, VEGF, B7-H3, Claudin18.2, TGF-β, Her2, IL-10, Siglec-15, Ras, C-myc, and the epitope B is the immunogenic epitope of the antigen B.

12. The recombinant polypeptide according to item 11, which is a bispecific antibody that can simultaneously bind to both the antigen or epitope A and the antigen or epitope B, preferably a humanized bispecific antibody or a bispecific antibody of complete human sequence.

13. The recombinant polypeptide according to any one of items 7 to 11, wherein,

the component A comprises: a light chain of the antibody, a VH+CH1 chain of the antibody fused with the In at the C-terminus, or a single-domain antibody variable region VHHa fused with the In at the C-terminus, optionally a tag protein is linked to the C-terminus of the In,

the component B comprises: a light chain of the antibody, a complete heavy chain of the antibody, and a Fc chain fused with the Ic at the N-terminus, or a single-domain antibody variable region VHHb fused with the Ic at the N-terminus, optionally a tag protein is linked to the N-terminus of the Ic, and the VHHa and the VHHb can be the same or different.

14. The recombinant polypeptide according to any one of items 3 to 13, wherein,

the tag protein is selected from the group consisting of Fc, His-tag, Strep-tag, Flag, HA and Maltose Binding Protein MBP.

15. A composition comprising the recombinant polypeptide according to any one of items 3 to 14.

16. A composition further comprising, in addition to the recombinant polypeptide according to any one of items 3 to 14, a carrier.

17. The composition according to item 16, which is a pharmaceutical composition, and the carrier is a pharmaceutically acceptable carrier.

18. A carrier, which is connected with the recombinant polypeptide according to any one of items 3 to 14, preferably for purification including chromatography.

19. A kit comprising the recombinant polypeptide according to any one of items 3 to 14, for the detection of the presence of the antigen or epitope A and/or the antigen or epitope B in a sample, wherein preferably the recombinant polypeptide is stored in a liquid or in a form of lyophilized powder, optionally can be present separately or in a state of being fixed to a carrier by linking, complexing, associating or chelating.

20. An expression vector, which is an expression vector for preparing the recombinant polypeptide according to any one of items 3 to 14.

21. A method for preparing recombinant polypeptides, comprising:

(1) providing a component A and a component B, wherein, the component A comprises a flanking sequence a, an N-terminal extein En and an In; the N-terminus of the flanking sequence a is connected to the C-terminus of the N-terminal extein En, and the C-terminus of the flanking sequence a is connected to the In, optionally a tag protein is further connected to the C-terminus of In;

the component B comprises a flanking sequence b, a C-terminal extein Ec and an Ic; the C-terminus of the flanking sequence b is connected to the N-terminus of the C-terminal extein Ec, and the N-terminus of the flanking sequence b is connected to the Ic, optionally a tag protein is connected to the N-terminus of Ic;

wherein, the flanking sequence a and the flanking sequence b are as described in items 1 or 2, and the coding sequences of the N-terminal extein En and the C-terminal extein Ec are derived from the same protein or different proteins; and

(2) performing an in vitro trans-splicing on the component A and the component B to obtain a recombinant polypeptide;

preferably, the step (1) comprises expressing the component A and the component B by a cell containing nucleic acid sequences encoding the component A and the component B; preferably, the N-terminal extein En and the C-terminal extein Ec can be different domains of an antibody.

22. The method for preparing recombinant polypeptides according to item 21, further comprising:

a first purification step of performing a chromatography on the component A and the component B before trans-splicing;

a second purification step of performing a chromatography on the recombinant polypeptide obtained by trans-splicing;

preferably, the chromatography method in the first purification step is selected from the group consisting of proteinA, proteinG, nickel column, Strep-Tactin affinity chromatography, anti-Flag antibody affinity chromatography, anti-HA antibody affinity chromatography and cross-linked starch affinity chromatography, and

preferably, the chromatography method in the second purification step is selected from an affinity chromatography method corresponding to the tag protein to remove unspliced components, or the unspliced components are removed by ion exchange, hydrophobic chromatography, or molecular sieve.

23. The method for preparing recombinant polypeptides according to item 21, wherein the recombinant polypeptide is a bispecific antibody, and the coding sequences of the bispecific antibody are derived from two different antibodies P and R, respectively;

1) splitting the antibody P into a En_{P} and a Ec_{P}, and designing the sequences of component A and component B; splitting the antibody R into a En_{R} and a Ec_{R}, and designing the component A′ and the component B′; wherein,

the component A comprises the flanking sequence a, the En_{P} and the In; the N-terminus of the flanking sequence a is connected to the C-terminus of the En_{P}, and the C-terminus of the flanking sequence a is connected to the In, optionally a tag protein is further connected to the C-terminus of In; the component B comprises the flanking sequence b, the Ec_{P} and the Ic; the C-terminus of the flanking sequence b is connected with the N-terminus of Ec_{P}, and the N-terminus of the flanking sequence b is connected with the Ic, optionally a tag protein is connected to the N-terminus of Ic;

the component A′ comprises the flanking sequence a, the En_{R} and the In; the N-terminus of the flanking sequence a is connected to the C-terminus of Ra, and the C-terminus of the flanking sequence a is connected to the In, optionally a tag protein is further connected to the C-terminus of In; the component B′ comprises the flanking sequence b, the Ec_{R} and the Ic; the C-terminus of the flanking sequence b is connected to the N-terminus of Ec_{R}, and the N-terminus of the flanking sequence b is connected to the Ic, optionally a tag protein is connected to the N-terminus of Ic;

2) performing a trans-splicing on the component A and the component B′, and/or the component A′ and the component B, to obtain the bispecific antibody.

24. A method of screening for a flanking sequence pair for a split intein, comprising:

1) splitting the amino acid sequence of protein P;

2) a flanking sequence a is an independently designed combination of 2-3 amino acids, denoted as flanking sequence a1-an, and a flanking sequence b is an independently designed combination of 2-3 amino acids, denoted as flanking sequence b1-bn; wherein, the amino acid is any amino acid selected from the group consisting of G, A, V, L, M, I, S, T, P, N, Q, F, Y, W, K, R, H, D, E, C;

3) for the split intein, expression sequences of components A1-An and components B1-Bn that contain the sequences split from protein P are designed by using the flanking sequences a1-an and b1-bn designed in step 2);

4) the expression sequences are linked to a vector respectively, and the components A and B are co-transfected in a manner of one-to-one correspondence and then intracellularly trans-spliced to obtain spliced products F1 to Fn;

5) detecting the spliced products F1 to Fn, and selecting the flanking sequence pair with a splicing efficiency more than 20%;

6) the flanking sequence pairs selected in 5) are analyzed, and the flanking sequences that can lead to free sulfhydryl group after splicing are removed to optimize the flanking sequence pair selected in 5);

7) the steps 1) to 5) are repeated to select the flanking sequence pairs 1 to m that have a splicing efficiency of top 20% in all candidate sequence pairs, and do not have free sulfhydryl groups in the recombinant polypeptide as the spliced product,

wherein, n is 2 or 3 and m is a positive integer.

25. The method of screening for a flanking sequence pair for a split intein according to item 24, further comprising:

1) splitting a protein R which is different from the protein P;

2) expression sequences of components A′1 to A′m and components B′1 to B′m are designed by using the flanking sequence pairs 1 to m;

3) the expression sequences are linked to a vector, and then a transfection, expression and purification are performed to obtain components A′1 to A′m and components B′1 to B′m,

4) the components A1-Am and the components B′1-B′m, and/or the components A′1-A′m and the components B1-Bm obtained by the flanking sequence pairs 1˜m are in vitro trans-spliced respectively in a manner of one-to-one correspondence; the spliced products are detected and multiple flanking sequence pairs with a splicing efficiency of more than 50% are selected.

26. A method for producing a recombinant polypeptide, characterized by performing a trans-splicing by using the flanking sequence pair for a split intein according to item 1 or 2.

27. Use of the flanking sequence pair for a split intein according to item 1 or 2 for the preparation of a recombinant polypeptide, preferably for the trans-splicing together with the split intein.

The advantages of recombinant polypeptides (such as, bispecific antibodies) prepared by the flanking sequences pair for a split intein of the present disclosure include: (1) no free sulfhydryl groups; (2) high-throughput and high-efficiency; and (3) the target product and impurities are easy to be distinguished and identified.

Definitions

It should be noted that the term “a” or “an” entity refers to one or more of that entity (entities); for example, “bispecific antibody” shall be understood to refer to one or more of bispecific antibody (antibodies). Likewise, the terms “a” (or “an”), “one or more” and “at least one” can be used interchangeably herein.

The term “polypeptide” as used herein includes the singular “polypeptide” as well as plural “polypeptides”, and also refers to a molecule composed of monomers (amino acids) linearly linked by amide bonds (also known as peptide bonds). A polypeptide may be derived from a natural biological source or may be produced by recombinant technology, but is not necessarily translated from a specified nucleic acid sequence. It may be generated in any manner, including by chemical synthesis.

As used herein, the term “recombinant” as it pertains to polypeptides or polynucleotides refers to a form of the polypeptide or polynucleotide that does not exist naturally, a non-limiting example of which can be achieved by combining polynucleotides or polypeptides that would not normally occur together.

“Homology” or “identity” or “similarity” refers to sequence similarity between two peptides or between two nucleic acid molecules. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to another sequence means that, when aligned, such percentage of bases (or amino acids) are the same in comparing the two sequences.

Biologically equivalent polynucleotides are polynucleotides that have the above-mentioned specified percentage of homology and encode polypeptides with the same or similar biologically activity.

The term “split intein” refers to a split intein consisting of two parts: an N-terminal protein splicing region or N-terminal fragment (i.e., In, or N′ fragment of intein) and a C-terminal protein splicing region or C-terminal fragment (i.e., Ic, or C fragment of intein). A gene expressing a precursor protein is split into two open reading frames, and the splitting site is internal to the intein sequence.

“N-terminal precursor protein” refers to a fusion protein translated by a fusion gene formed by a N-terminal extein (En)-encoding gene and a N-terminal fragment (In)-encoding gene.

“C-terminal precursor protein” refers to a fusion protein translated by a fusion gene formed by a C-terminal fragment (Ic)-encoding gene and a C-terminal extein (Ec)-encoding gene.

The N-terminal fragment (In) or the C-terminal fragment (Ic) of the split intein alone does not have a protein splicing function. After protein translation, the In in the N-terminal precursor protein and the Ic in the C-terminal precursor protein bind to each other by a non-covalent bond to form a functional intein, which can catalyze protein trans-splicing reaction, thus two separate protein exons are connected by peptide bonds (the N-terminal protein exon or N-terminal extein can be referred to as En, and the C-terminal protein exon or C-terminal extein can be referred to as Ec) (Ozawa. T. Nat Biotechbol. 21 (2003) 287 93).

Protein trans-splicing refers to a protein splicing reaction mediated by split inteins. During the trans-splicing process, firstly, the N-terminal fragment (In) and the C-terminal fragment (Ic) of the split intein recognize each other and are non-covalently bound. Once bound, the structure is correctly folded and the split intein has a reconstructed active center, and then the protein splicing reaction is completed according to the typical protein splicing pathway, thereby linking the exteins at both sides.

The term “In” refers to a separate N-terminal portion of the split-intein, and also can be referred to herein as the N-terminal fragment or N-terminal protein splicing region of the split-intein.

The term “Ic” refers to a separate C-terminal portion of the split intein, and also can be referred to herein as the C-terminal fragment or C-terminal protein splicing region of the split intein.

The term “flanking sequence a” refers to an amino acid sequence flanking both the N-terminus of In and the C-terminus of En and linking the In and the En. As shown in FIG. 5, the first amino acid next to the N-terminus of the In is defined as position −1, the second amino acid residue next to the N-terminus of the In is defined as position −2, and the third amino acid residue next to the N-terminus of the In is defined as position −3, and so on until reaching the En. Generally speaking, the core sequences of the flanking sequence a are at positions −1 and −2, which are directly related to splicing efficiency.

The term “flanking sequence b” refers to an amino acid sequence flanking both the C-terminus of Ic and the N-terminus of Ec and linking the Ic and the Ec. As shown in FIG. 5, the first amino acid residue next to the C-terminus of Ic is defined as position +1, the second amino acid residue next to the C-terminus of Ic is defined as position +2, and the third amino acid residue next to the C-terminus of Ic is defined as position +3, and so on until reaching the Ec. In general, the core sequences of the flanking sequence b are at positions +1 and +2, which are directly related to splicing efficiency.

During the split intein-mediated trans-splicing, for example as shown in FIG. 5, the In and the flanking sequence a are separated, and the Ic and the flanking sequence b are separated, and then the flanking sequence a and the flanking sequence b are linked, whereby the En and the Ec linked to corresponding flanking sequence are connected. As a result, the amino acid residue at position −1 of the flanking sequence a and the amino acid residue at position +1 of the flanking sequence b are directly linked by a peptide-bond, and the amino acid at position −1 is located at the N-terminal of the amino acid at position +1.

In the present disclosure, 20 common amino acids (hereinafter referred to as 20 amino acids) are used for the screening of flanking sequences, that is, glycine (G), alanine (A), valine (V), leucine (L), methionine (M), isoleucine (I), serine (S), threonine (T), proline (P), asparagine (N), glutamine (Q), phenylalanine (F), tyrosine (Y), tryptophan (W), lysine (K), arginine (R), histidine (H), aspartic acid (D), glutamic acid (E) and cysteine (C).

As used herein, an “antibody” or “antigen-binding polypeptide” refers to a polypeptide or a polypeptide complex that specifically recognizes and binds to an antigen or immunogenic epitope.

An antibody can be an intact antibody and any antigen binding fragment or a single chain thereof. Thus the term “antibody” includes any protein or peptide containing a specific molecule, wherein the specific molecule comprises at least a portion of an immunoglobulin molecule having biological activity of binding to an antigen or immunogenic epitope. Examples of such include, but are not limited to a complementary determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The term “antibody fragment” or “antigen-binding fragment”, as used herein, refers a portion of an antibody. The term “antibody fragment” includes aptamers, spiegelmers, and diabodies. The term “antibody fragment” also includes any synthetic or genetically engineered protein that acts like an antibody by binding to a specific antigen or immunogenic epitope to form a complex.

A “single-chain variable fragment” or “scFv” refers to a fusion protein of the variable regions of the heavy (VH) and light chains (VL) of immunoglobulins.

The term “antibody” encompasses a wide variety of polypeptides that can be biochemically recognized. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG or IgE, respectively. The immunoglobulin subclasses (isotypes) e.g., IgG1, IgG2, IgG3, IgG4, IgG5, etc. are well characterized and functionally specific. Modified versions of each of these classes and isotypes are readily discernible to those skilled in the art in view of the present disclosure and, accordingly, are within the scope of the present disclosure.

All immunoglobulin classes are clearly within the scope of the present disclosure, the following discussion will generally be directed to the IgG class of immunoglobulin molecules.

With regard to IgG, a standard immunoglobulin molecule comprises two identical light chain polypeptides with a molecular weight of approximately 23,000 Daltons, and two identical heavy chain polypeptides with a molecular weight of 53,000-70,000 joined by disulfide bonds in a “Y” configuration.

Antibodies, antigen-binding polypeptides, variants or derivatives thereof in the present disclosure include, but are not limited to, polyclonal, monoclonal, multispecific, human, humanized, primatized, or chimeric antibodies, single chain antibodies, antigen-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies. Immunoglobulin or antibody molecules of the disclosure can be of any type (e.g., IgG, IgE, IgM, IgD, IgA, and IgY), any class (e.g., IgG1, IgG2, IgG3, IgG4, IgA1 and IgA2) or any subclass of immunoglobulin molecule.

In some examples, for example, certain immunoglobulins derived from camelid species or engineered based on camelid immunoglobulins, an intact immunoglobulin molecule thereof may consist of only heavy chains without light chains. See, for example, Hamers-Casterman et al., Nature 363:446-448 (1993).

Both the light and heavy chains are divided into structural regions and functional homology regions. The terms “constant” and “variable” are used functionally. In this regard, it will be appreciated that the variable domains of both the light (VL) and heavy (VH) chain determine the antigen recognition and specificity. Generally, the number of the constant region domains increases as they become more distal from the antigen-binding site or amino-terminus of the antibody. The N-terminal portion is a variable region and the C-terminal portion is a constant region; the CH3 and CL domains actually comprise the carboxy-terminus of the heavy and light chain, respectively.

Regarding the antigen-binding site, those skilled in the art can easily identify the amino acids of the CDR and framework regions for any given heavy chain or light chain variable region since they have been clearly defined (see, “Sequences of Proteins of Immunological Interest,” Kabat, E., et al., U.S. Department of Health and Human Services, (1983); Chothia and Lesk, J. MoI. Biol., 196:901-917 (1987), the full text of which is incorporated herein by reference).

In the case where there are two or more definitions of a term that are used and/or accepted within the art, the definitions of the term as used herein are intended to include all meanings, unless explicitly stated to the contrary.

The term “complementarity determining region” (“CDR”) refers to the non-contiguous antigen binding sites present in the variable regions of both heavy chain and light chain polypeptides. This specific region has been described by Kabat et al., U.S. Department of Health and Human Services, “Sequences of Proteins of Immunological Interest” (1983) and by Chothia et al., J. MoI. Biol. 196:901-917 (1987), the full text of which is incorporated herein by reference. Those skilled in the art can routinely determine which residues comprise a particular CDR if the amino acid sequence of the variable region of the antibody is provided.

The “Kabat numbering” as used herein refers to the numbering system described by Kabat et al., U.S. Department of Health and Human Services, “Sequence of Proteins of Immunological Interest” (1983).

The term “heavy chain constant region” as used herein includes amino acid sequences derived from immunoglobulin heavy chains. A polypeptide comprising a heavy chain constant region comprises at least one of the following: a CH1 domain, a hinge (for example, upper hinge region, middle hinge region, and/or lower hinge region) domain, a CH2 domain, a CH3 domain, or a variant or fragment thereof. For example, the antigen-binding polypeptide for use in the present disclosure may comprise a polypeptide chain comprising a CH1 domain; a polypeptide comprising a CH1 domain, at least a portion of a hinge domain and a CH2 domain; a polypeptide chain comprising a CH1 domain and a CH3 domain; a polypeptide chain comprising a CH1 domain, at least a portion of a hinge domain and a CH3 domain, or a polypeptide chain comprising a CH1 domain, at least a portion of a hinge domain, a CH2 domain, and a CH3 domain. In another embodiment, the polypeptide of the present disclosure comprises a polypeptide chain comprising a CH3 domain. In addition, the antibodies used in the present disclosure may lack at least a portion of a CH2 domain (for example, all or a portion of the CH2 domain). As set forth above, it will be understood by those skilled in the art that the heavy chain constant regions may be modified so that they differ in amino acid sequence from naturally occurring immunoglobulin molecules.

The heavy chain constant regions of the antibody disclosed herein can be derived from different immunoglobulin molecules. For example, the heavy chain constant region of a polypeptide may include a CH1 domain derived from an IgG1 molecule and a hinge region derived from an IgG3 molecule. In another example, the heavy chain constant region may include a hinge region that is partly derived from an IgG1 molecule and partly from an IgG3 molecule. In another example, the heavy chain portion may comprise a chimeric hinge that is partly derived from an IgG1 molecule and partly derived from an IgG4 molecule.

The term “light chain constant region” as used herein includes an amino acid sequence derived from the light chain of an antibody. Preferably, the light chain constant region includes at least one of a constant kappa domain and a constant lambda domain.

The term “VH domain” includes the amino-terminal variable domain of an immunoglobulin heavy chain, and the term “CH1 domain” includes a first (mostly amino-terminal) constant region of an immunoglobulin heavy chain. The CH1 domain is adjacent to the VH domain and is the amino terminus of the hinge region of the immunoglobulin heavy chain molecule.

The term “CH2 domain” as used herein includes a portion of a heavy chain molecule that ranges, for example, from a residue at about position 244 to a residue at position 360 of an antibody according to a conventional numbering system (residues at position 244 to 360, according to Kabat numbering system; and residues at position 231-340, according to EU numbering system; see Kabat et al., U.S. Department of Health and Human Services, “Sequences of Proteins of Immunological Interest” (1983). The CH2 domain is unique because it does not pair with another domain tightly. On the contrary, two N-linked branched carbohydrate chains are inserted between the two CH2 domains of an intact natural IgG molecule. It is documented that the CH3 domain extends from the CH2 domain to the C-terminus of the IgG molecule, and comprises about 108 residues.

By “specifically binding” or “specific to”, it generally means that when the antibody binds to the antigen epitope, the binding via the antigen-binding domain is easier than that via binding to a random, unrelated antigen epitope. The term “specificity” is used herein to determine the affinity of a certain antibody to bind to a particular antigen epitope.

The term “treating” (“treat” or “treatment”) as used herein refers to both therapeutic treatment and prophylactic or preventive measures, wherein the object is to prevent or slow down (lessen) an undesired physiological change or disorder, such as cancer progression. Beneficial or desired clinical outcomes include, but are not limited to, alleviating symptoms, diminishing the degree of disease, stabilizing (for example, preventing it from worsening) disease state, delaying or slowing the disease progression, alleviating or palliating the disease state, and alleviating (whether partial or total), regardless of whether detectable or undetectable. “Treatment” can also mean prolonging survival as compared to expected survival without receiving treatment.

Any of the aforementioned antibodies or polypeptides may further include additional polypeptides, for example, an encoded polypeptide as described herein, a signal peptide at the N-terminus of the antibody used to direct secretion, or other heterologous polypeptides as described herein.

In other embodiments, the polypeptide of the present disclosure may comprise conservative amino acid substitutions.

A “conservative amino acid substitution” is one in which an amino acid residue is substituted by an amino acid residue having a similar side chain Families of amino acid residues having similar side chains have been defined in the art, including basic side chains (e.g., lysine, arginine, histidine), acidic side chains (e.g., aspartic acid, glutamic acid), uncharged polar side chains (for example, glycine, asparagine, glutamine, serine, threonine, tyrosine, cysteine), non-polar side chains (for example, alanine, valine, leucine, isoleucine, proline, phenylalanine, methionine, tryptophan), β-branched side chains (for example, threonine, valine, isoleucine) and aromatic side chains (e.g. tyrosine, phenylalanine, tryptophan, histidine). Therefore, non-essential amino acid residues of immunoglobulin polypeptides are preferably substituted by other amino acid residues from the same side chain family. In another embodiment, a string of amino acids may be substituted by a structurally similar string of amino acids that differs in sequence and/or composition of the side chain family

Transient transfection is a technical means of introducing DNA into eukaryotic cells. In transient transfection, recombinant DNA is introduced into a highly infectious cell line to obtain transient but high-level expression of the gene of interest. The transfected DNA does not have to be integrated into the host chromosome, and the transfected cells can be harvested in a shorter time than stable transfection, and the target product in the expression supernatant can be detected.

### BRIEF DESCRIPTION OF THE DRAWINGS

FIG. 1 is a schematic diagram (A) of split intein-mediated splicing of homologous polypeptide fragments and a schematic diagram (B) of the protein primary structure of each component. (Pa, N-terminal fragment of the split protein P; In, N-terminal fragment of the split intein; Pb, C-terminal fragment of the split protein P; Ic, C-terminal fragment of the split intein; TAG, tag protein; FS, flanking sequence).

FIG. 2 is a schematic diagram (A) of split intein-mediated splicing of heterologous polypeptide fragments and a schematic diagram (B) of the protein primary structure of each component. (Pa, N-terminal fragment of the split protein P; Ra, N-terminal fragment of the split protein R; In, N-terminal fragment of the split intein; Pb, C-terminal fragment of the split protein P; Rb, C-terminal fragment of the split protein R; TAG, tag protein; Ic, C-terminal fragment of the split intein; FS, flanking sequence).

FIG. 3 is a schematic diagram (A) of split intein-mediated antibody splicing in vitro and a schematic diagram (B) of the protein primary structure of each component, wherein the spliced product is a bispecific antibody. (C) is an exemplary schematic diagram of the amino acid sequence near the split intein-mediated antibody splicing site, “X” indicates that the amino acid at that position is any amino acid or deletion. (LC, light chain; HC, heavy chain; TAG, tag protein; FS, flanking sequence).

FIG. 4 is a schematic diagram (A) for the construction of an expression plasmid for the component A of bispecific antibody, and a schematic diagram (B) for the construction of an expression plasmid for the component B.

FIG. 5 is a schematic diagram of flanking sequence numbering. (TAG, tag protein; FS, flanking sequence).

FIG. 6 shows the detection results of reducing SDS-PAGE and coomassie brilliant blue staining of the expression supernatants of 293E cells after proteinA affinity purification, wherein the 293E cells are co-transfected with expression plasmids corresponding to different inteins and different flanking sequences. FIG. 6 (A) to (E) shows the detection results after purification of cell supernatants of component A and component B co-transfected with different inteins based on different flanking sequences, respectively.(MW, molecular weight)

FIG. 7 shows the results of non-reducing SDS-PAGE and coomassie brilliant blue staining of the purified products of component A and component B′ with different inteins expressed by 293E cells, respectively. (A) Detection results of purified products of Fab5, Fab9 and Fab11; (B) Detection results of purified products of HAb5, HAb9 and HAb11.

FIG. 8 shows the detection of non-reducing SDS-PAGE and coomassie brilliant blue staining of the spliced products of component A and component B′ with different inteins, wherein (A) the intein is IMPDH-1, the flanking sequence a is GGG, and the flanking sequence b is SI; (B) the intein is PhoRadA, the flanking sequence a is GK, and the flanking sequence b is THT. In FIGS. 8(A) and (B), the spliced product 1 means that the DTT is added before mixing the components A and B; the spliced product 2 means that the DTT is added after mixing the components A and B′; the reduced (ie., RD) means that the DTT is added; the non-reduced (ie., NON-RD) means that no DTT is added; the “non-splicing” indicates that components A and B′ are mixed without adding DTT. In FIG. 8(C), the intein is PhoRadA, the flanking sequence a is GK, the flanking sequence b is THT. “SPLICING 1” and “NON-SPLICING 1” refer to reaction systems containing the component A and component B′ at concentrations of 5 μM and 4 μM, respectively, as well as 2 mM DTT; “SPLICING 2” and “NON-SPLICING 2” refer to reaction systems containing the component A and component B′ with concentrations of 10 μM and 1 μM, respectively, as well as 2 mM DTT; “SPLICING 3” and “NON-SPLICING 3” refer to reaction systems containing the component A and component B′ with concentrations of 5 μM and 1 μM, respectively, as well as 2 mM DTT; wherein “SPLICING 1” to “SPLICING 3” are incubated overnight at 37° C., and “NON-SPLICING 1” to “NON-SPLICING 3” are incubated at 4° C. overnight; the control bands are Fab11 (non-reduced) for component A, and HAb11 (non-reduced) for component B′, and mAb. (RD, reduced; MW, molecular weight; mAb, monoclonal antibody)

FIG. 9 shows the detection result of spliced product by double antigen sandwich ELISA in which the intein is IMPDH-1, the flanking sequence a is GGG, and the flanking sequence b is SI; wherein, the coating antigen is CD38, and the detection antigen is horseradish peroxidase (HRP)-labeled PD-L1.

FIG. 10 shows the base peak ion (BPI) map of Fab5+HAb5 (spliced product 1) after digestion. (A) BPI map of Fab5+HAb5 (spliced product 1) after trypsin digestion; (B) BPI map of Fab5+HAb5 (spliced product 1) after chymotrypsin digestion; (C) BPI map of Fab5+HAb5 (spliced product 1) after Glu-C digestion.

FIG. 11 shows the SDS-PAGE and coomassie staining detection after co-transfection expression and affinity purification of component A and component B by applying intein PhoRadA and IMPDH-1 to human IgG2, IgG3 or IgG4 subclasses. (RD, reduced; MW, molecular weight)

### DETAILED DESCRIPTION OF THE INVENTION

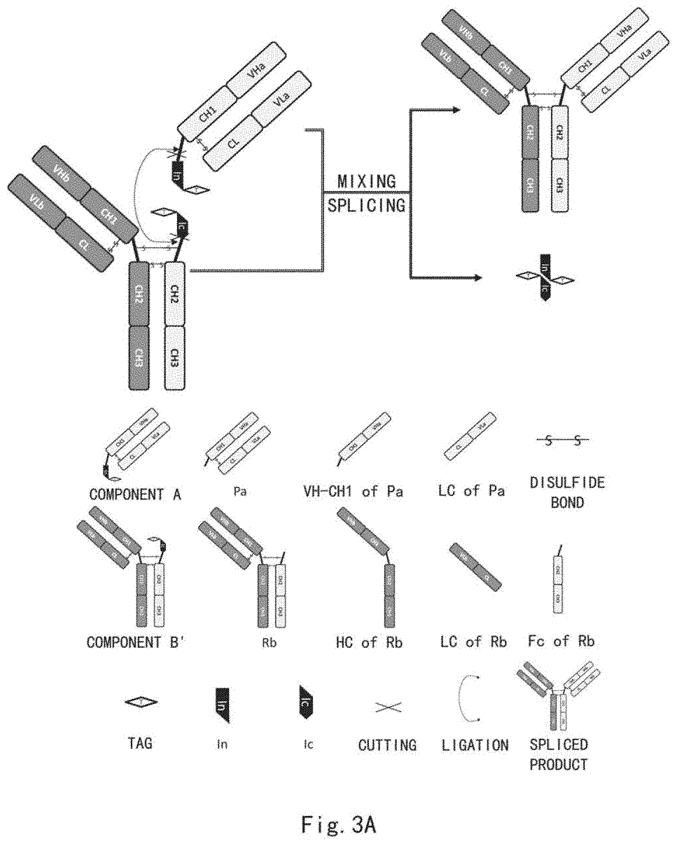

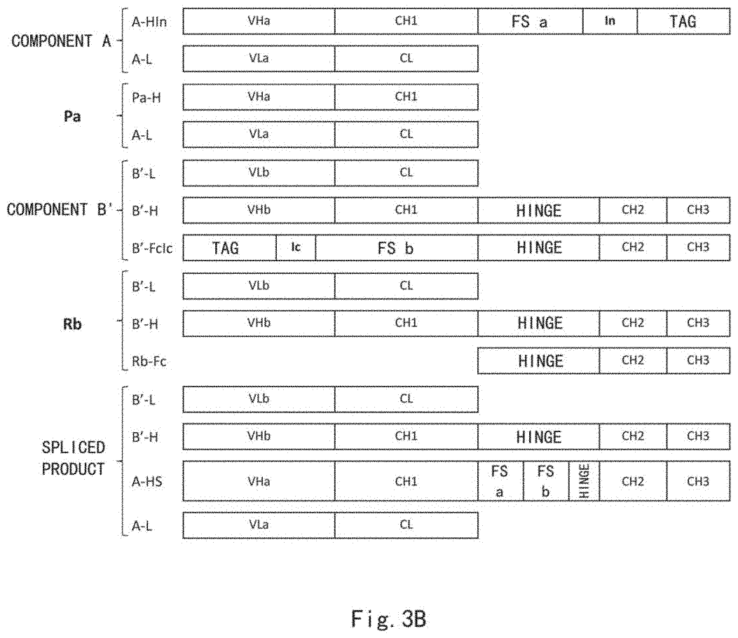

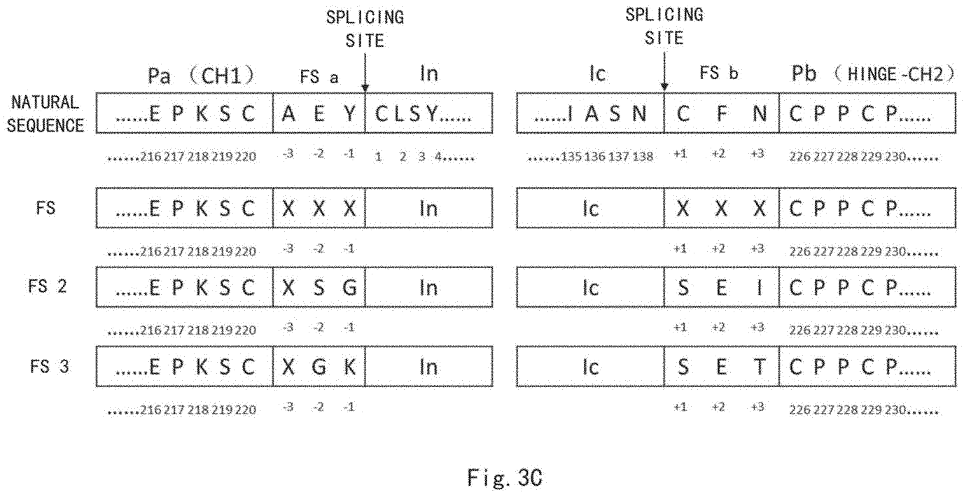

The present disclosure relates to a preparation method of a bispecific antibody, which includes: splitting the DNA sequence of the target antibody, constructing a mammalian cell expression vector through whole gene synthesis, purifying the vector, and then the purified vector can be transiently transfected or stably transfected into mammalian cells such as HEK293 or CHO, respectively. The fermentation broth is collected separately, and the component A and the component B are purified by methods such as protein A, protein L, nickel column, Strep-Tactin affinity chromatography, anti-Flag antibody affinity chromatography, anti-HA antibody affinity chromatography or cross-linked starch affinity chromatography; the purified component A and component B are subjected to in vitro trans-splicing, and the spliced product is subjected to affinity chromatography for tag proteins such as nickel column to obtain a bispecific antibody with high-purity. The process flow is shown in FIG. 3A.

The antibodies described herein can be from any animal origin, including birds and mammals. Preferably, the antibodies are human, murine, donkey, rabbit, goat, guinea pig, camel, llama, horse or chicken antibodies. In another embodiment, the variable region may be derived from a condricthoid (e.g., from a shark).

In some embodiments, the antibody may be conjugated to therapeutic agents, prodrugs, peptides, proteins, enzymes, viruses, lipids, biological response modifiers, pharmaceutical agents, or PEG.

The antibody may be linked or fused to a therapeutic agent, which may include detectable labels, such as radioactive labels, immunomodulators, hormones, enzymes, oligonucleotides, photoactive therapeutic or diagnostic agents, cytotoxicity agents, which can be drugs or toxins, ultrasound enhancers, non-radioactive labels, a combination thereof and other such components known in the art.

The antibody can be detectably labeled by coupling it to chemiluminescent compounds. Then, the presence of the chemiluminescent-labeled antigen-binding polypeptide is determined by detecting the luminescence produced during the chemical reaction. Examples of particularly useful chemiluminescent labeling compounds are luminol, isoluminol, theromatic acridinium ester, imidazole, acridinium salt and oxalate ester.

The antibodies can also be detectably labeled by using fluorescence emitting metals such as 152Eu, or other lanthanide labels. These metals can be attached to the antibody by using the following metal chelating groups, such as diethylenetriaminepentaacetic acid (DTPA) or ethylenediaminetetraacetic acid (EDTA).

The binding specificity of the antigen-binding polypeptides of the present disclosure can be measured by in vitro experiments, such as immunoprecipitation, radioimmunoassay (RIA) or enzyme-linked immunosorbent assay (ELISA).

Cell lines for production of recombinant polypeptides can be selected and cultured by using techniques well known to those skilled in the art.

Standard techniques known to those skilled in the art can be used to introduce mutations into the nucleotide sequences encoding the antibodies of the present disclosure, including, but not limited to, site-directed mutagenesis and PCR-mediated mutations which result in amino acid substitutions. Preferably, the variants (including derivatives), relative to the reference variable heavy chain region, CDR-H1, CDR-H2, CDR-H3, light chain variable region, CDR-L1, CDR-L2 or CDR-L3, encode less than 50 amino acid substitutions, less than 40 amino acid substitutions, less than 30 amino acid substitutions, less than 25 amino acid substitutions, less than 20 amino acid substitutions, less than 15 amino acid substitutions, and less than 10 amino acid substitutions, less than 5 amino acid substitutions, less than 4 amino acid substitutions, less than 3 amino acid substitutions, or less than 2 amino acid substitutions. Alternatively, mutations can be randomly introduced along all or part of the encoding sequence, for example, by saturation mutagenesis, and the resulting mutants can be screened for biological activity to identify mutations that retain activity.

The tag protein used in the present disclosure may be Fc, oligo-histidine (His-tag), Strep-tag, Flag, HA, or maltose-binding protein (MBP) or the like.

The transfection used in the present disclosure may be transient transfection or stable transfection.

Mammalian cells such as HEK293 or CHO are used in the present disclosure, but are not limited thereto.

Liquids containing expression products from mammalian cells, such as fermentation broth and culture medium supernatant, can be purified by methods such as protein A, protein G, nickel column, Strep-Tactin affinity chromatography, anti-Flag antibody affinity chromatography, anti-HA antibody affinity chromatography or cross-linked starch affinity chromatography.

The spliced product can be subjected to affinity chromatography for the tag protein to remove unspliced components.

The gene fragment used for constructing the vector of the present disclosure can be constructed by whole gene synthesis, but is not limited thereto.

The vector used in the present disclosure is pcDNA3.1 or pCHO1.0, but is not limited thereto.

The restriction enzymes used in the present disclosure include, but are not limited to, NotI, NruI, or BamHI-HF, for example.

BLAST is an alignment program that uses default parameters. Specifically, the programs are BLASTN and BLASTP. Detailed information of these programs is available at the following Internet address: http://www.ncbi.nlm.nih.gov/blast/Blast.cgi.

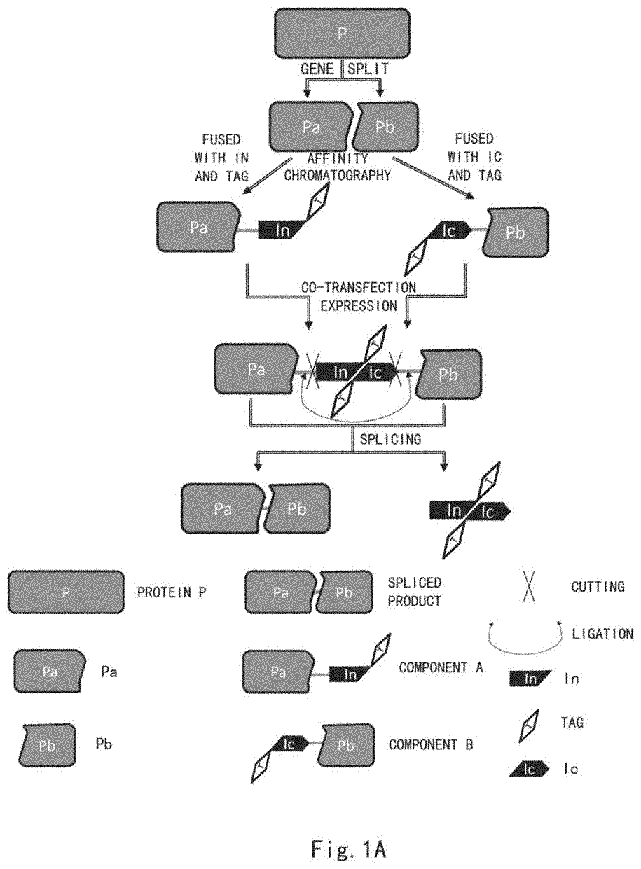

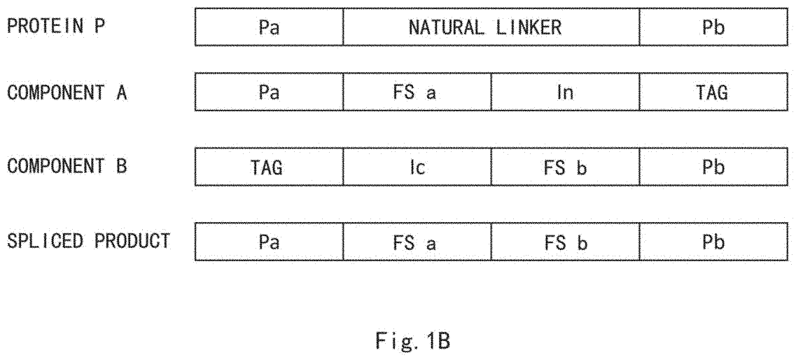

In a specific embodiment of the present disclosure, as shown in FIGS. 1, 2, and 3, a component A expression plasmid (pPa-FSa-In-Tag) and a component B expression plasmid (pTag-Ic-FSb-Pb) or component A′ expression plasmid (pRa-FSa-In-Tag) and component B′ expression plasmid (pTag-Ic-FSb-Rb) can be constructed.

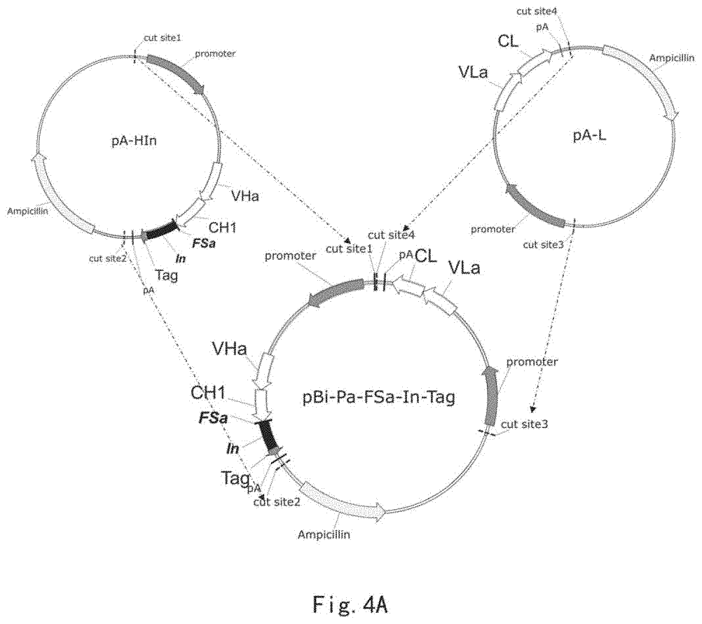

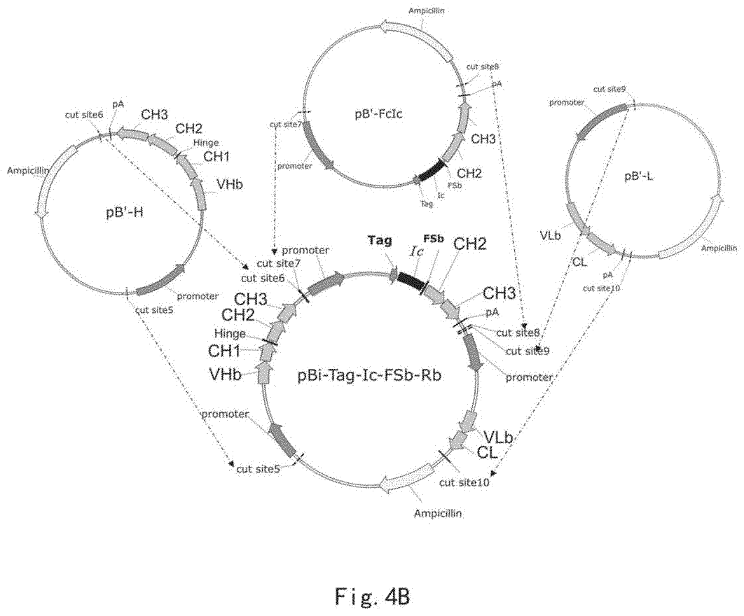

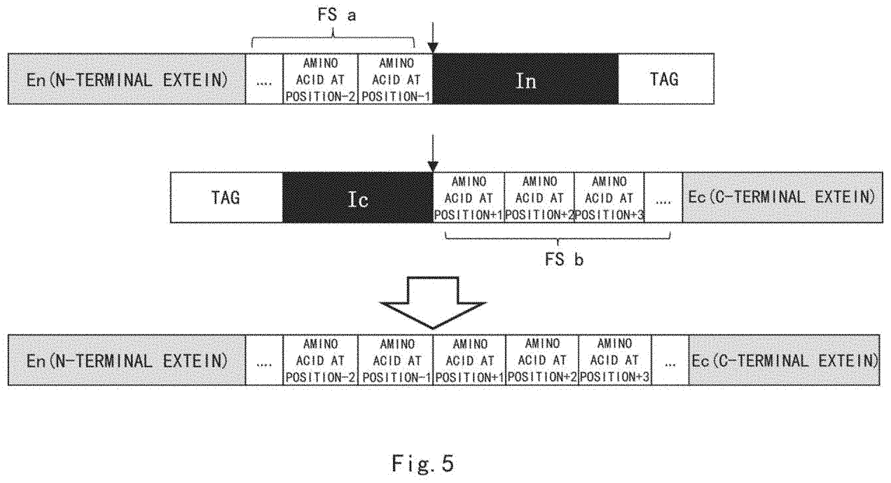

In another specific embodiment of the present disclosure, as shown in FIGS. 4A and 4B, the Pa-HIn and Pa-L can be constructed into the same plasmid, namely component A expression plasmid (pBi-Pa-FSa-In-Tag); or the pB′-L, pB′-H and pB′-FcIc can be constructed into the same plasmid, namely component B′ expression plasmid (pBi-Tag-Ic-FSb-Rb) by molecular cloning methods such as enzyme cleavage and enzyme ligation.

In another specific embodiment of the present disclosure, the component B expression plasmids may include three types of expression plasmids, pB-L, pB-H, and pB-FcIc.

In the present disclosure, Pa also refers to the N-terminal protein exon or N-terminal extein of protein P, also referred to as Enp; Pb also refers to the C-terminal protein exon or C-terminal extein of protein P, also referred to as Ecp. Ra also refers to the N-terminal protein exon or N-terminal extein of protein R, also referred to as En_{R}; Rb also refers to the C-terminal protein exon or C-terminal extein of protein R, also referred to as Ec_{R}.

TABLE 1
Amino acid sequences of some polypeptides involved in the present disclosure

| SEQ | | |
|---|---|---|
| ID | | |
| NO | Gene name(Source) | Amino acid sequence |
| 1 | Human CD38 | VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQ |
| | (Source: UniProtKB-P28907) | RDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQ |
| | | TLEAWVIHGGREDSRDLCQDPTIKELESIISKRNIQFSCKNIYRPDKFLQCVKNPEDSSCTSEI |
| 2 | Human BCMA | MLQMAGQCSQNEYFDSLLHACIPCQLRCSSNTPPLTCQRYCNASVTNSVKGTNA |
| | (Source: UniProtKB-Q02223) | |
| 3 | Human CTLA-4 | MHVAQPAVVLASSRGIASFVCEYASPGKATEVRVTVLRQADSQVTEVCAATYMMGNELTFLDDSICTGTSSGNQVNLTIQGLRAMDTGLYICKVELM |
| | (Source: UniProtKB-P16410) | YPPPYYLGIGNGTQIYVIDPEPCPDSD |
| 4 | Human LAG-3 | VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDER |
| | (Source: UniProtKB-P18627) | GRQRGDFSLWLRPARRADAGEYRAAVHLRDRALSCRLRLRLGQASMTASPPGSLRASDWVILNCSFSRPDRPASVHWFRNRGQGRVPVRESPHHHLA |
| | | ESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLR |
| | | LEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQ |
| | | GERLLGAAVYFTELSSPGAQRSGRAPGALPAGHL |
| 5 | Human TIGIT | MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTY |
| | (Source: UniProtKB-Q495A1) | TGRIFLEVLESSVAEHGARFQIP |
| 6 | Human PD-1 | PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRND |
| | (Source: UniProtKB-Q15116) | SGTYLCGAISLAPKAQIKESLRAELRVTERRAEVPTAHPSPSPRPAGQFQTLV |
| 7 | Human PD-L1 | FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCM |
| | (Source: UniProtKB-Q9NZQ7) | ISYGGADYKRITVKVNAPYNKINQRILVVDPVTSEHELTCQAEGYPKAEVIWTSSDHQVLSGKTTTTNSKREEKLFNVTSTLRINTTTNEIFYCTFR |
| | | RLDPEENHTAELVIPELPLAHPPNER |
| 8 | Human SLAMF7 | SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQE |
| | (Source: UniProtKB-Q9NQ25) | YVLHVYEHLSKPKVTMGLQSNKNGTCVTNLTCCMEHGEEDVIYTWKALGQAANESHNGSILPISWRWGESDMTFICVARNPVSRNFSSPILARKLCE |
| | | GAADDPDSSM |
| 9 | Human CEA | KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVN |
| | (Source: UniProtKB-P06731) | EEATGQFRVYPELPKPSISSNNSKPVEDKDAVAFTCEPETQDATYLWWVNNQSLPVSPRLQLSNGNRTLTLFNVTRNDTASYKCETQNPVSARRSDS |
| | | VILNVLYGPDAPTISPLNTSYRSGENLNLSCHAASNPPAQYSWFVNGTFQQSTQELFIPNITVNNSGSYTCQAHNSDTGLNRTTVTTITVYAEPPKP |
| | | FITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISP |
| | | SYTYYRPGVNLSLSCHAASNPPAQYSWLIDGNIQQHTQELFISNITEKNSGLYTCQANNSASGHSRTTVKTITVSAELPKPSISSNNSKPVEDKDAV |
| | | AFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCH |
| | | SASNPSPQYSWRINGIPQQHTQVLFIAKITPNNNGTYACFVSNLATGRNNSIVKSITVSASGTSPGLSA |
| 10 | Human CD3ϵ | DGNEEMGGITQTPYKVSISGTTVILTCPQYPGSEILWQHNDKNIGGDEDDKNIGSDEDHLSLKEFSELEQSGYYVCYPRGSKPEDANFYLYLRARVC |
| | (Source: UniProtKB-P07766) | ENCMEMD |
| 11 | Human CD16A | GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQA |
| | (Source: UniProtKB-P08637) | PRWVFKEEDPIHLRCHSWKNTALHKVTYLQNGKGRKYFHHNSDFYIPKATLKDSGSYFCRGLFGSKNVSSETVNITITQGLAVSTISSFFPPGYQ |
| 12 | Human TGF-β1 | ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPK |
| | (Source: UniProtKB-P01137) | VEQLSNMIVRSCKCS |
| 13 | Human TGF-β2 | ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPK |
| | (Source: UniProtKB-P61812) | IEQLSNMIVKSCKCS |
| 14 | Human TGF-β3 | ALDTNYCFRNLEENCCVRPLYIDFRQDLGWKWVHEPKGYYANFCSGPCPYLRSADTTHSTVLGLYNTLNPEASASPCCVPQDLEPLTILYYVGRTPK |
| | (Source: UniProtKB-P10600) | VEQLSNMVVKSCKCS |
| 15 | Human VEGFA | APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFL |
| | (Source: UniProtKB-P15692) | QHNKCECRPKKDRARQEKKSVRGKGKGQKRKRKKSRYKSWSVYVGARCCLMPWSLPGPHPCGPCSERRKHLFVQDPQTCKCSCKNTDSRCKARQLEL |
| | | NERTCRCDKPRR |
| 16 | Human IL-10 | PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENL |
| | (Source: UniProtKB-P22301) | KTLRLRLRRCHRFLPCENKSKAVEQVKNAFNKLQEKGIYKAMSEFDIFINYIEAYMTMKIRN |
| 17 | Human CD20 (Source: UniProtKB- | MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIIS |
| | P11836) | GSLLAATEKNSRKCLVKGKMIMNSLSLFAAISGMILSIMDILNIKISHFLKMESLNFIRAHTPYINIYNCEPANPSEKNSPSTQYCYSIQSLFLGIL |
| | | SVMLIFAFFQELVIAGIVENEWKRTCSRPKSNIVLLSAEEKKEQTIEIKEEVVGLTETSSQPKNEEDIEIIPIQEEEEEETETNFPEPPQDQESSPI |
| | | ENDSSP |
| 18 | Human Claudin18.2 | MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVS |
| | (Source: UniProtKB-P56856) | IFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCI |
| | | ACRGLAPEETNYKAVSYHASGHSVAYKPGGFKASTGFGSNTKNKKIYDGGARTEDEVQSYPSKHDYV |
| 19 | Human FIXa (Source: UniProtKB- | YNSGKLEEFVQGNLERECMEEKCSFEEAREVFENTERTTEFWKQYVDGDQCESNPCLNGGSCKDDINSYECWCPFGFEGKNCELDVTCNIKNGRCEQ |
| | P00740) | FCKNSADNKVVCSCTEGYRLAENQKSCEPAVPFPCGRVSVSQTSKLTRAETVFPDVDYVNSTEAETILDNITQSTQSFNDFTRVVGGEDAKPGQFPW |
| | | QVVLNGKVDAFCGGSIVNEKWIVTAAHCVETGVKITVVAGEHNIEETEHTEQKRNVIRIIPHHNYNAAINKYNHDIALLELDEPLVLNSYVTPICIA |
| | | DKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEE |
| | | CAMKGKYGIYTKVSRYVNWIKEKTKLT |
| 20 | Human FX (Source: UniProtKB- | ANSFLEEMKKGHLERECMEETCSYEEAREVFEDSDKTNEFWNKYKDGDQCETSPCQNQGKCKDGLGEYTCTCLEGFEGKNCELFTRKLCSLDNGDCD |
| | P00742) | QFCHEEQNSVVCSCARGYTLADNGKACIPTGPYPCGKQTLERRKRSVAQATSSSGEAPDSITWKPYDAADLDPTENPFDLLDFNQTQPERGDNNLTR |
| | | IVGGQECKDGECPWQALLINEENEGFCGGTILSEFYILTAAHCLYQAKRFKVRVGDRNTEQEEGGEAVHEVEVVIKHNRFTKETYDFDIAVLRLKTP |
| | | ITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFK |
| | | DTYFVTGIVSWGEGCARKGKYGIYTKVTAFLKWIDRSMKTRGLPKAKSHAPEVITSSPLK |
| 21 | Human HER2 (Source: UniProtKB- | TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDN |
| | P04626) | GDPLNNTTPVTGASPGGLRELQLRSLTEILKGGVLIQRNPQLCYQDTILWKDIFHKNNQLALTLIDTNRSRACHPCSPMCKGSRCWGESSEDCQSLT |
| | | RTVCAGGCARCKGPLPTDCCHEQCAAGCTGPKHSDCLACLHFNHSGICELHCPALVTYNTDTFESMPNPEGRYTFGASCVTACPYNYLSTDVGSCTL |
| | | VCPLHNQEVTAEDGTQRCEKCSKPCARVCYGLGMEHLREVRAVTSANIQEFAGCKKIFGSLAFLPESFDGDPASNTAPLQPEQLQVFETLEEITGYL |
| | | YISAWPDSLPDLSVFQNLQVIRGRILHNGAYSLTLQGLGISWLGLRSLRELGSGLALIHHNTHLCFVHTVPWDQLFRNPHQALLHTANRPEDECVGE |
| | | GLACHQLCARGHCWGPGPTQCVNCSQFLRGQECVEECRVLQGLPREYVNARHCLPCHPECQPQNGSVTCFGPEADQCVACAHYKDPPFCVARCPSGV |
| | | KPDLSYMPIWKFPDEEGACQPCPINCTHSCVDLDDKGCPAEQRASPLT |
| 22 | Human IL-10R | HGTELPSPPSVWFEAEFFHHILHWTPIPNQSESTCYEVALLRYGIESWNSISNCSQTLSYDLTAVTLDLYHSNGYRARVRAVDGSRHSNWTVTNTRF |
| | (Source: UniProtKB-Q13651) | SVDEVTLTVGSVNLEIHNGFILGKIQLPRPKMAPANDTYESIFSHFREYEIAIRKVPGNFTFTHKKVKHENFSLLTSGEVGEFCVQVKPSVASRSNK |
| | | GMWSKEECISLTRQYFTVTN |
| 23 | EGFP (Source: UniProtKB- | MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQER |
| | A0A076FL24) | TIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPV |
| | | LLPDNHYLSTQSALSKDPNEKRDHMVLLEFVTAAGITLGMDELYK |

TABLE 2
Amino acid sequences of some tag proteins

| SEQ ID | | |
|---|---|---|
| NO | Tag protein name | Amino acid sequence |
| 24 | His-tag | HHHHHHH |
| | (Oligo-histidine) | |
| 25 | Flag | DYKDDDDK |
| 26 | HA | YPYDVPDYA |
| 27 | C-MYC | EQKLISEEDL |
| 28 | St rep-tag | WSHPQFEK |
| 29 | Avi-tag | GLNDIFEAQKIEWHE |
| 30 | Fc | PCPAPELLGGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |

TABLE 3
In and 1c sequences of some split inteins

| SEQ | | | SEQ | | |
|---|---|---|---|---|---|
| ID | Intein | | ID | Intein | |
| NO | name | In | NO | name | Ic |
| 31 | SspDnaE | CLSFGTEILTVEYGPLPIGKIVSEEINCSVYSV | 32 | SspDnaE | MVKVIGRRSLGVQRIFDIGLPQDHNFLLANGAIAAN |
| | | DPEGRVYTQAIAQWHDRGEQEVLEYELEDGSVI | | | |
| | | RATSDHRFLTTDYQLLAIEEIFARQLDLLTLEN | | | |
| | | IKQTEEALDNHRLPFPLLDAGTIK | | | |
| 33 | SspDnaB | CISGDSLISLASTGKRVSIKDLLDEKDFEIWAI | 34 | SspDnaB | SPEIEKLSQSDIYWDSIVSITETGVEEVFDLTVPGPHNFVA |
| | | NEQTMKLESAKVSRVFCTGKKLVYILKTRLGRT | | | NDIIVHN |
| | | IKATANHRFLTIDGWKRLDELSLKEHIALPRKL | | | |
| | | ESSSLQ | | | |
| 35 | MxeGyrA | CITGDALVALPEGESVRIADIVPGARPNSDNAI | 36 | MxeGyrA | GKPEFAPTTYTVGVPGLVRFLEAHHRDPDAQAIADELTDGR |
| | | DLKVLDRHGNPVLADRLFHSGEHPVYTVRTVEG | | | FYYAKVASVTDAGVQPVYSLRVDTADHAFITNGFVSHN |
| | | LRVTGTANHPLLCLVDVAGVPTLLWKLIDEIKP | | | |
| | | GDYAVIQRSAFSVDCAGFAR | | | |
| 37 | MjaTFIIB | SVDYNEPIIIKENGEIKVVKIGELIDKIIENSE | 38 | MjaTFIIB | NSDFIFLKIKEINKVEPTSGYAYDLTVPNAENFVAGFGGFV |
| | | NIRREGILEIAKCKGIEVIAFNSNYKFKFMPVS | | | LHN |
| | | EVSRHPVSEMFEIVVEGNKKVRVTRSHSVFTIR | | | |
| | | DNEVVPIRVDELKVGDILVLAK | | | |
| 39 | PhoVMA | CVSGDTPVLLDAGERRIGDLFMEAIRPKERGEI | 40 | PhoVMA | MHISGVFDVYDLMVPDYGYNFIGGNGLIVLHN |
| | | GQNEEIVRLHDSWRIYSMVGSEIVETVSHAIYH | | | |
| | | GKSNAIVNVRTENGREVRVTPVHKLFVKIGNSV | | | |
| | | IERPASEVNEGDEIAWPSVSENGDSQTVTTTLV | | | |
| | | LTFDRVVSKE | | | |
| 41 | TvoVMA | CVSGETPVYLA | 42 | TvoVMA | DGKTIKIKDLYSSERKKEDNIVEAGSGEEIIHLKDPIQIYS |
| | | | | | YVDGTIVRSRSRLLYKGKSSYLVRIETIGGRSVSVTPVHKL |
| | | | | | FVLTEKGIEEVMASNLKVGDMIAAVAESESEARDCGMSEEC |
| | | | | | VMEAEVYTSLEATFDRVKSIAYEKGDFDVYDLSVPEYGRNF |
| | | | | | IGGEGLLVLHN |
| 43 | Gp41-1 | CLDLKTQVQTPQGMKEISNIQVGDLVLSNTGYN | 44 | Gp41-1 | MMLKKILKIEELDERELIDIEVSGNHLFYANDILTHN |
| | | EVLNVFPKSKKKSYKITLEDGKEIICSEEHLFP | | | |
| | | TQTGEMNISGGLKEGMCLYVKE | | | |
| 45 | Gp41-8 | CLSLDTMVVTNGKAIEIRDVKVGDWLESECGPV | 46 | Gp41-8 | MCEIFENEIDWDEIASIEYVGVEETIDINVTNDRLFFANGI |
| | | QVTEVLPIIKQPVFEIVLKSGKKIRVSANHKFP | | | LTHN |
| | | TKDGLKTINSGLKVGDFLRSRAK | | | |
| 47 | IMPDH-1 | CFVPGTLVNTENGLKKIEEIKVGDKVFSHTGKLQ | 48 | IMPDH-1 | MKFKLKEITSIETKHYKGKVHDLTVNQDHSYNVRGTVVHN |
| | | EVVDTLIFDRDEEIISINGIDCTKNHEFYVIDKE | | | |
| | | NANRVNEDNIHLFARWVHAEELDMKKHLLIELE | | | |
| 49 | PhoRadA | CFARDTEVYYENDTVPHMESIEEMYSKYASMNGE | 50 | PhoRadA | NGYAVPLDNVFVYTLDIASGEIKKTRASYIYREKVEKLIEI |
| | | LPFD | | | KLSSGYSLKVTPSHPVLLFRDGLQWVPAAEVKPGDVVVGVR |
| | | | | | EEVLRRRIISKGELEFHEVSSVRIIDYNNWVYDLVIPETHN |
| | | | | | FIAPNGLVLHN |

TABLE 4
Flanking sequences a of some split inteins

| SEQ | | Amino acid sequences of |
|---|---|---|
| ID NO | No. | flanking sequence a |
| 51 | FSa1 | AEY |
| 52 | FSa2 | SG |
| 53 | FSa3 | GS |
| 54 | FSa4 | MGG |
| 55 | FSa5 | RY |
| 56 | FSa6 | TY |
| 57 | FSa7 | GK |
| 58 | FSa8 | NR |
| 59 | FSa9 | GGG |
| 60 | FSa10 | DK |
| 61 | FSa11 | GY |
| 62 | FSa12 | XX* |
| 63 | FSa13 | XXX* |
| 202 | FSa14 | DKG |
| 203 | FSa15 | DKT |

*X represents any amino acid selected from the 20 amino acids (A, D, E, F, G, H, I, K, L, M, N, P, Q, R, S, T, V, W, Y, C) defined in the present disclosure.

TABLE 5
Flanking sequences b of some split inteins

| SEQ | | Amino acid sequences of |
|---|---|---|
| ID NO | No. | flanking sequence b |
| 64 | FSb1 | CFN |
| 65 | FSb2 | SVY |
| 66 | FSb3 | SIE |
| 67 | FSb4 | TEA |
| 68 | FSb5 | TIH |
| 69 | FSb6 | TVI |
| 70 | FSb7 | SSS |
| 71 | FSb8 | SAV |
| 72 | FSb9 | SI |
| 73 | FSb10 | TQL |
| 74 | FSb11 | SEI |
| 75 | FSb12 | SEH |
| 76 | FSb13 | SET |
| 77 | FSb14 | THT |
| 78 | FSb15 | XX* |
| 79 | FSb16 | XXX* |
| 204 | FSb17 | ST |

*X represents any amino acid selected from the 20 amino acids (A, D, E, F, G, H, I, K, L, M, N, P, Q, R, S, T, V, W, Y, C) defined in the present disclosure.

TABLE 6
Some amino acid sequences and sequence No. of the En domains involved in the construction of component A or A′

| SEQ | | | |
|---|---|---|---|
| ID | | | |
| NO | Domain | Code | Amino acid sequences |
| 168 | Hinge | Hin1 | DKTHT |
| 169 | Hinge | Hin2 | EKCCVE |
| 170 | Hinge | Hin3 | GDTTHTCPRCPEPKSCDTPPPCPRCPEPKSCDTPPPCPRCPEPKSCDTPPPCPR |
| 171 | Hinge | Hin4 | YGPP |
| 172 | CL | Lc1 | RTVAAPSVFIFPPSDEQLKSGTASVVCLLNNFYPREAKVQWKVDNALQSGNSQESVTEQDSKDSTYSLSSTLTLSKADYEKHKVYACEVT |
| | | | HQGLSSPVTKSFNRGEC |
| 173 | CL | Lc2 | GQPKANPTVTLFPPSSEELQANKATLVCLISDFYPGAVTVAWKADGSPVKAGVETTKPSKQSNNKYAASSYLSLTPEQWK |
| | | | SHRSYSCQVTHEGSTVEKTVAPTECS |
| 174 | CL | Lc3 | GQPKAAPSVTLFPPSSEELQANKATLVCLISDFYPGAVTVAWKADSSPVKAGVETTTPSKQSNNKYAASSYLSLTPEQWKS |
| | | | HRSYSCQVTHEGSTVEKTVAPTECS |
| 175 | CL | Lc4 | GQPKAAPSVTLFPPSSEELQANKATLVCLISDFYPGAVTVAWKADSSPAKAGVETTTPSKQSNNKYAASSYLSLTPEQWKS |
| | | | HRSYSCQVTHEGSTVEKTVAPTECS |
| 176 | CL | Lc5 | GQPKAAPSVTLFPPSSEELQANKATLVCLISDFYPGAVKVAWKADGSPVNTGVETTTPSKQSNNKYAASSYLSLTPEQWKS |
| | | | HRSYSCQVTHEGSTVEKTVAPAECS |
| 177 | CL | Lc6 | GQPKAAPTVTLFPPSSEELQANKATLVCLISDFYPGAVKVAWKADSSPAKAGVETTTPSKQSNNKYAASSYLSLTPEQWKS |
| | | | HRSYSCQVTHEGSTVEKTVAPTECS |
| 178 | CL | Lc7 | VAAPSVFIFPPSDEQLKSGTASVVCLLNNFYPREAKVQWKVDNALQSGNSQESVTEQDSKDSTYSLSSTLTLSKADYEKHK |
| | | | VYACEVTHQGLSSPVTKSFNRGEC |
| 179 | CH1 | G1CH1 | ASTKGPSVFPLAPSSKSTSGGTAALGCLVKDYFPEPVTVSWNSGALTSGVHTFPAVLQSSGLYSLSSVVTVPSSSLGTQTYICNVNHKPSN |
| | | | TKVDKKVEPKSC |
| 180 | CH1 | G2CH1 | ASTKGPSVFPLAPCSRSTSESTAALGCLVKDYFPEPVTVSWNSGALTSGVHTFPAVLQSSGLYSLSSVVTVPSSNFGTQ |
| | | | TYTCNVDHKPSNTKV |
| 181 | CH1 | G3CH1 | ASTKGPSVFPLAPCSRSTSGGTAALGCLVKDYFPEPVTVSWNSGALTSGVHTFPAVLQSSGLYSLSSVVTVPSSSLGTQ |
| | | | TYTCNVNHKPSNTKVDKRVE |
| 182 | CH1 | G4CH1 | ASTKGPSVFPLAPCSRSTSESTAALGCLVKDYFPEPVTVSWNSGALTSGVHTFPAVLQSSGLYSLSSVVTVPSSSLGTKT |
| | | | YTCNVDHKPSNTKVDKR |
| 198 | Pa | CD38-Pa | VPRWRQQWSGPGTTKRFPETVLARCVKYTEIHPEMRHVDCQSVWDAFKGAFISKHPCNITEEDYQPLMKLGTQTVPCNKILLWSRIKDL |
| | | | AHQFTQVQRDMFTLEDTLLGYLADDLTWCGEFNTSKINYQSCPDWRKDCSNNPVSVFWKTVSRRFAEAACDVVHVMLNGSRSKIFDKN |
| | | | STF |
| 200 | Pa | GFP-Pa | MVSKGEELFTGVVPILVELDGDVNGHKFSVSGEGEGDATYGKLTLKFICTTGKLPVPWPTLVTTLTYGVQCFSRYPDHMKQHDFFKSAM |
| | | | PEGYVQERTIFFKDDGNYKTRAEVKFEGDTLVNRIELKGIDFKEDGNILGHKLEYNYNSHNVYIMADKQKNGIKVNFKIRHNIED |

TABLE 7
Some amino acid sequences and sequence numbers of the Ec domains involved in the construction of
component B or B′

| SEQ | | | |
|---|---|---|---|
| ID | | | |
| NO | Domain | Code | Amino acid sequences |
| 183 | CH2 | G1CH2 | CPPCPAPELLGGPSVFLFPPKPKDTLMISRTPEVTCVVVDVSHEDPEVKFNWYVDGVEVHNAKTKPREEQYNSTYR |
| | | | VVSVLTVLHQDWLNGKEYKCKVSNKALPAPIEKTISKAK |
| 184 | CH2 | G2CH2 | CPPCPAPPVAGPSVFLFPPKPKDTLMISRTPEVTCVVVDVSHEDPEVQFNWYVDGVEVHNAKTKPREEQFNSTFRV |
| | | | VSVLTVVHQDWLNGKEYKCKVSNKGLPAPIEKTISKTK |
| 185 | CH2 | G2DCH2 | CPPCPAPPVAGPSVFLFPPKPKDTLMISRTPEVTCVVVDVSHEAPEVQFNWYVDGVEVHNAKTKPREEQFNSTFRV |
| | | | VSVLTVVHQDWLNGKEYKCKVSNKGLPAPIEKTISKTK |
| 186 | CH2 | G3CH2 | CPAPELLGGPSVFLFPPKPKDTLMISRTPEVTCVVVDVSHEDPEVQFKWYVDGVEVHNAKTKPREEQYNSTFRVV |
| | | | SVLTVLHQDWLNGKEYKCKVSNKALPAPIEKTISKTK |
| 187 | CH2 | G4CH2 | CPSCPAPEFLGGPSVFLFPPKPKDTLMISRTPEVTCVVVDVSQEDPEVQFNWYVDGVEVHNAKTKPREEQFNSTYR |
| | | | VVSVLTVLHQDWLNGKEYKCKVSNKGLPSSIEKTISKAK |
| 188 | CH3 | G1CH3 | GQPREPQVYTLPPSRDELTKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGK |
| 189 | CH3 | G2CH3 | GQPREPQVYTLPPSREEMTKNQVSLTCLVKGFYPSDISVEWESNGQPENNYKTTPPMLDSDGSFFLYSKLTVDKSR |
| | | | WQQGNVFSCSVMHEALHNHYTQKSLSLSPGK |
| 190 | CH3 | G3CH3 | GQPREPQVYTLPPSREEMTKNQVSLTCLVKGFYPSDIAVEWESSGQPENNYNTTPPMLDSDGSFFLYSKLTVDKSR |
| | | | WQQGNIFSCSVMHEALHNRFTQKSLSLSPGK |
| 191 | CH3 | G4CH3 | GQPREPQVYTLPPSQEEMTKNQVSLTCLVKGFYPSDIAVEWESNGQPENNYKTTPPVLDSDGSFFLYSRLTVDKSR |
| | | | WQEGNVFSCSVMHEALHNHYTQKSLSLSLGK |
| 192 | CH3 | G1CH3- | GQPREPQVYTLPPCRDELTKNQVSLWCLVKGFYPSDIAVEWESNGQPENNYKTTPPVLDSDGSFFLYSKLTVDKSR |
| | | CW | WQQGNVFSCSVMHEALHNHYTQKSLSLSPGK |
| 193 | CH3 | G1CH3- | GQPREPQVCTLPPSRDELTKNQVSLSCAVKGFYPSDIAVEWESNGQPENNYKTTPPVLDSDGSFFLVSKLTVDKSR |
| | | CSAV | WQQGNVFSCSVMHEALHNHYTQKSLSLSPGK |
| 194 | CH3 | G1CH3-W | GQPREPQVYTLPPSRDELTKNQVSLWCLVKGFYPSDIAVEWESNGQPENNYKTTPPVLDSDGSFFLYSKLTVDKSR |
| | | | WQQGNVFSCSVMHEALHNHYTQKSLSLSPGK |
| 195 | CH3 | G1CH3- | GQPREPQVYTLPPSRDELTKNQVSLSCAVKGFYPSDIAVEWESNGQPENNYKTTPPVLDSDGSFFLVSKLTVDKSR |
| | | SAV | WQQGNVFSCSVMHEALHNHYTQKSLSLSPGK |
| 196 | CH3 | G1CH3-V | GQPREPQVYTLPPSRDELTKNQVSLTCLVKGFYPSDIAVEWESNGQPENNYKTTPPVLDSDGSFFLVSKLTVDKSR |
| | | | WQQGNVFSCSVMHEALHNHYTQKSLSLSPGK |
| 197 | CH3 | G1CH3-RF | GQPREPQVYTLPPSRDELTKNQVSLTCLVKGFYPSDIAVEWESNGQPENNYKTTPPVLDSDGSFFLYSKLTVDKSR |
| | | | WQQGNVFSCSVMHEALHNRFTQKSLSLSPGK |
| 199 | Pb | CD38-Pb | EVHNLQPEKVQTLEAWVIHGGREDSRDLCQDPTIKELESIISKRNIQFSCKNIYRPDKFLQCVKNPEDSSCTSEI |
| 201 | Pb | EGFP-Pb | VQLADHYQQNTPIGDGPVLLPDNHYLSTQSALSKDPNEKRDHMVLLEFVTAAGITLGMDELYK |

TABLE 8
Variable region sequences of anti-CD3 antibody

| | Amino acid sequences of anti-CD3 antibody variable region (Bold and | | | |
|---|---|---|---|---|
| | underlined amino acids are CDR regions) | | | |
| Anti- | | SEQ | | SEQ |
| body | | ID | | ID |
| code | VH | NO | VL | NO |
| 2a5 | QVQLVESGGGVVQPGRSLRLSCAASGFTFSTYAMNWV | 80 | QTVVTQEPSLTVSPGGTVTLTCRSSTGAVTTSNYANW | 81 |
| | RQAPGKGLEWVARIRSKYNNYATYYADSVKDRFTISR | | VQQKPGQAPRGLIGGTNKRAPGVPARFSGSLLGGKAA | |
| | DDSKNTLYLQMNSLRAEDTAVYYCARHGNFGNSYVSW | | LTLSGVQPEDEAEYYCALWYSNLWVFGGGTKVEIK | |
| | FAYWGQGTLVTVSS | | | |
| 2j5a | QVQLVESGGGVVQPGRSLRLSCAASGFTFSTYAMNWV | 82 | QTVVTQEPSLTVSPGGTVTLTCRSSTGAVTTSNYANW | 83 |
| | RQAPGKGLEWVARIRSKYNNYATYYADSVKDRFTISR | | FQQKPGQAPRGLIGGTNKRAPGVPARFSGSLLGGKAA | |
| | DDSKNTLYLQMNSLRAEDTAVYYCARHGNFGNSYVSW | | LTLSGVQPEDEAEYYCALWYSNLWVFGGGTKVEIK | |
| | AAYWGQGTLVTVSS | | | |

TABLE 9
Variable region sequences of anti-B7-H3 antibody

| | Amino acid sequences of anti-B7-H3 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| 8H9 | QVQLQQSGAELVKPGASVKLSCKASGYTFTNYDINW | 84 | DIVMTQSPATLSVTPGDRVSLSCRASQSISDYLH | 85 |
| (Cancer Research | VRQRPEQGLEWIGWIFPGDGSTQYNEKFKGKATLTT | | WYQQKSHESPRLLIKYASQSISGIPSRFSGSGSG | |
| 61, 4048-4054, | DTSSSTAYMQLSRLTSEDSAVYFCARQTTATWFAYW | | SDFTLSINSVEPEDVGVYYCQNGHSFPLTFGAGT | |
| May 15, 2001) | GQGTLVTVSS | | KLELK | |
| BRCA69D | QVQLQQSGAELARPGASVKLSCKASGYTFTSYWMQW | 86 | DIQMTQTTSSLSASLGDRVTISCRASQDISNYLN | 87 |
| (US20120294796A1) | VKQRPGQGLEWIGTIYPGDGDTRYTQKFKGKATLTA | | WYQQKPDGTVKLLIYYTSRLHSGVPSRFSGSGSG | |
| | DKSSSTAYMQLSSLASEDSAVYYCARRGIPRLWYFD | | TDYSLTIDNLEQEDIATYFCQQGNTLPPTFGGGT | |
| | VWGAGTTVTVSS | | KLEIK | |

TABLE 10
Variable region sequences of anti-CD38 antibody

| | Amino acid sequence of anti-CD38 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| Dara | EVQLLESGGGLVQPGGSLRLSCAVSGFTFNSFAMSWVRQ | 88 | EIVLTQSPATLSLSPGERATLSCRASQSVSSYLAW | 89 |
| (US9040050) | APGKGLEWVSAISGSGGGTYYADSVKGRFTISRDNSKNT | | YQQKPGQAPRLLIYDASNRATGIPARFSGSGSGTD | |
| | LYLQMNSLRAEDTAVYFCAKDKILWFGEPVFDYWGQGTL | | FTLTISSLEPEDFAVYYCQQRSNWPPTFGQGTKVE | |
| | VTVSS | | IK | |
| MOR | QVQLVESGGGLVQPGGSLRLSCAASGFTFSSYYMNWVRQ | 90 | DIELTQPPSVSVAPGQTARISCSGDNLRHYYVYWY | 91 |
| (US8088896) | APGKGLEWVSGISGDPSNTYYADSVKGRFTISRDNSKNT | | QQKPGQAPVLVIYGDSKRPSGIPERFSGSNSGNTA | |
| | LYLQMNSLRAEDTAVYYCARDLPLVYTGFAYWGQGTLVT | | TLTISGTQAEDEADYYCQTYTGGASLVFGGGTKLT | |
| | VSS | | VLGQ | |
| 2F5 | QVQLVQSGAEVKKPGSSVKVSCKASGGTFSSYAFSWVRQ | 92 | DIQMTQSPSSLSASVGDRVTITCRASQGISSWLAW | 93 |
| (US9040050) | APGQGLEWMGRVIPFLGIANSAQKFQGRVTITADKSTST | | YQQKPEKAPKSLIYAASSLQSGVPSRFSGSGSGTD | |
| | AYMDLSSLRSEDTAVYYCARDDIAALGPFDYWGQGTLVT | | FTLTISSLQPEDFATYYCQQYNSYPRTFGQGTKVE | |
| | VSS | | IK | |

TABLE 11
Variable region sequences of anti-EpCAM antibody

| | Amino acid sequences of anti-EpCAM antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| 3-171 | QVQLVQSGAEVKKPGSSVKVSCKASGGTFSSYAIS | 94 | EIVMTQSPATLSVSPGERATLSCRASQSVSSNLAWYQ | 95 |
| (US20100310463 | WVRQAPGQGLEWMGGIIPIFGTANYAQKFQGRVTI | | QKPGQAPRLIIYGASTTASGIPARFSASGSGTDFTLT | |
| | TADESTSTAYMELSSLRSEDTAVYYCARGLLWNYW | | ISSLQSEDFAVYYCQQYNNWPPAYTFGQGTKLEIK | |
| | GQGTLVTVSS | | | |
| 2-6 | EVQLVESGPELKKPGETVKISCKASGYTFTDYSMHW | 96 | DIQMTQSPSSLSASLGERVSLTCRASQEISVSLSWLQ | 97 |
| (TW102107344) | VKQAPGKGLKWMGWINTETGEPTYADDFKGRFAFSL | | QEPDGTIKRLIYATSTLDSGVPKRFSGSRSGSDYSLT | |
| | ETSASTAYLQINNLKNEDTATYFCARTAVYWGQGTT | | ISSLESEDFVDYYCLQYASYPWTFGGGTKLEIK | |
| | VTVSS | | | |

TABLE 12
Variable region sequences of anti-BCMA antibody

| | Amino acid sequence of anti-BCMA antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| B50 | QVQLVQSGAEVKKPGASVKVSCKASGYSFPDYYIN | 98 | DIVMTQTPLSLSVTPGQPASISCKSSQSLVHSNGNTYLH | 99 |
| (US9598500) | WVRQAPGQGLEWMGWIYFASGNSEYNQKFTGRVTM | | WYLQKPGQSPQLLIYKVSNRFSGVPDRFSGSGSGTDFTL | |
| | TRDTSINTAYMELSSLTSEDTAVYFCASLYDYDWY | | KISRVEAEDVGIYYCSQSSIYPWTFGQGTKLEIK | |
| | FDVWGQGTMVTVSS | | | |
| B140153 | QVQLVQSGAEVKKPGSSVKVSCKASGGTFSSYAIS | 100 | LPVLTQPPSASGTPGQRVTISCSGRSSNIGSNSVNWYRQ | 101 |
| | WVRQAPGQGLEWMGRIIPILGIANYAQKFQGRVTI | | LPGAAPKLLIYSNNQRPPGVPVRFSGSKSGTSASLAISG | |
| (WO2016090320A1) | TADKSTSTAYMELSSLRSEDTAVYYCARGGYYSHD | | LQSEDEATYYCATWDDNLNVHYVFGTGTKVTVLG | |
| | MWSEDWGQGTLVTVSS | | | |
| B69 | QLQLQESGPGLVKPSETLSLTCTVSGGSISSGSYF | 102 | SYVLTQPPSVSVAPGQTARITCGGNNIGSKSVHWYQQPP | 103 |
| | WGWIRQPPGKGLEWIGSIYYSGITYYNPSLKSRVT | | GQAPVVVVYDDSDRPSGIPERFSGNSNGNTATLTISRVE | |
| (US2017051068A1) | ISVDTSKNQFSLKLSSVTAADTAVYYCARHDGAVA | | AGDEAVYYCQVWDSSSDHVVFGGGTKLTVL | |
| | GLFDYWGQGTLVTVSS | | | |

TABLE 13
Variable region sequences of anti-CTLA-4 antibody

| | Amino acid sequences of anti-CTLA-4 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| Yervoy | QVQLVESGGGVVQPGRSLRLSCAASGFTFSSYTMH | 104 | EIVLTQSPGTLSLSPGERATLSCRASQSVGSSYLA | 105 |
| (US20020086014A1) | WVRQAPGKGLEWVTFISYDGNNKYYADSVKGRFTI | | WYQQKPGQAPRLLIYGAFSRATGIPDRFSGSGSGT | |
| | GTLVTVSSSRDNSKNTLYLQMNSLRAEDTAIYYCA | | FTLTISRLEPEDFAVYYCQQYGSSPWTFGQGTKV | |
| | RTGWLGPFDYWGQ | | VEIK | |

TABLE 14
Variable region sequences of anti-TIGIT antibody

| | Amino acid sequence of anti-TIGIT antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| 10A7 | EVQLVESGGGLTQPGKSLKLSCEASGFTFSSFTMH | 106 | DIVMTQSPSSLAVSPGEKVTMTCKSSQSLYYSGV | 107 |
| (US20090258013A1) | WVRQSPGKGLEWVAFIRSGSGIVFYADAVRGRFTI | | KENLLAWYQQKPGQSPKLLIYYASIRFTGVPDRF | |
| | SRDNAKNLLFLQMNDLKSEDTAMYYCARRPLGHNT | | TGSGSGTDYTLTITSVQAEDMGQYFCQQGINNPL | |
| | FDSWGQGTLVTVSS | | TFGDGTKLEIK | |
| MAB10 | QVQLQESGPGLVKPSQTLSLTCTVSGGSIESGLYYWG | 108 | EIVLTQSPGTLSLSPGERATLSCRASQSVSSSYLA | 109 |
| (WO2017059095A1) | WIRQPPGKGLEWIGSIYYSGSTYYNPSLKSRATISVD | | WYQQKPGQAPRLLIYGASSRATGIPDRFSGSGSGT | |
| | TSKNQFSLKLSSVTAADTAVYYCARDGVLALNKRSFD | | DFTLTISRLEPEDFAVYYCQQHTVRPPLTFGGGTK | |
| | IWGQGTMVTVSS | | VEIK | |

TABLE 15
Variable region sequences of anti-LAG-3 antibody

| | Amino acid sequence of anti-LAG-3 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| LAG35 | QVQLQQWGAGLLKPSETLSLTCAVYGGSFSDYYWN | 110 | EIVLTQSPATLSLSPGERATLSCRASQSISSYLA | 111 |
| (US9505839B2) | WIRQPPGKGLEWIGEINHRGSTNSNPSLKSRVTLS | | WYQQKPGQAPRLLIYDASNRATGIPARFSGSGSG | |
| | LDTSKNQFSLKLRSVTAADTAVYYCAFGYSDYEYN | | TDFTLTISSLEPEDFAVYYCQQRSNWPLTFGQGT | |
| | WFDPWGQGTLVTVSS | | NLEIK | |
| L3E3 | EVQLLESGAEVKKPGASVKVSCKASGYTFTSYYMH | 112 | QSVLTQPASASGSPGQSITISCTGTSSDVGGYNY | 113 |
| (US9902772B2) | WVRQAPGQGLEWMGIINPSAGSTSYAQKFQGRVTM | | VSWYQQHPGKAPKLMIYDVSNRPSGVSNRFSGSK | |
| | TRDTSTSTVYMELSSLRSEDTAVYYCARELMATGG | | SGNTASLTISGLQAEDEANYYCSSYTSSSTNVFG | |
| | FDYWGQGTLVTVSS | | TGTKVTVL | |

TABLE 16
Variable region sequences of anti-PD-1 antibody

| | Amino acid sequences of anti-PD-1 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| 5C4 | QVQLVESGGGVVQPGRSLRLDCKASGITFSNSGMH | 114 | EIVLTQSPATLSLSPGERATLSCRASQSVSSYLA | 115 |
| (WO2006121168) | WVRQAPGKGLEWVAVIWYDGSKRYYADSVKGRFTI | | WYQQKPGQAPRLLIYDASNRATGIPARFSGSGSG | |
| | SRDNSKNTLFLQMNSLRAEDTAVYYCATNDDYWGQ | | TDFTLTISSLEPEDFAVYYCQQSSNWPRTFGQGT | |
| | GTLVTVSS | | KVEIK | |
| H409A11 | QVQLVQSGVEVKKPGASVKVSCKASGYTFTNYYMY | 116 | EIVLTQSPATLSLSPGERATLSCRASKGVSTSGY | 117 |
| (WO2008156712A1) | WVRQAPGQGLEWMGGINPSNGGTNFNEKFKNRVTL | | SYLHWYQQKPGQAPRLLIYLASYLESGVPARFSG | |
| | TTDSSTTTAYMELKSLQFDDTAVYYCARRDYRFDM | | SGSGTDFTLTISSLEPEDFAVYYCQHSRDLPLTF | |
| | GFDYWGQGTTVTVSS | | GGGTKVEIK | |

TABLE 17
variable region sequences of anti-PD-Ll antibody

| | Amino acid sequences of anti-PD-1 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| S70 | EVQLVESGGGLVQPGGSLRLSCAASGFT | 118 | DIQMTQSPSSLSASVGDRVTITCRASQDVSTAVAWYQQKPGKAP | 119 |
| (WO2010077634A1) | FSDSWIHWVRQAPGKGLEWVAWISPYGG | | KLLIYSASFLYSGVPSRFSGSGSGTDFTLTISSLQPEDFATYYC | |
| | STYYADSVKGRFTISADTSKNTAYLQMN | | QQYLYHPATFGQGTKVEIK | |
| | SLRAEDTAVYYCARRHWPGGFDYWGQGT | | | |
| | LVTVSS | | | |
| 12A4 | QVQLVQSGAEVKKPGSSVKVSCKTSGDT | 120 | EIVLTQSPATLSLSPGERATLSCRASQSVSSYLAWYQQKPGQAP | 121 |
| (US7943743B2) | FSTYAISWVRQAPGQGLEWMGGIIPIFG | | RLLIYDASNRATGIPARFSGSGSGTDFTLTISSLEPEDFAVYYC | |
| | KAHYAQKFQGRVTITADESTSTAYMELS | | QQRSNWPTFGQGTKVEIK | |
| | MSLRSEDTAVYFCARKFHFVSGSPFGDV | | | |
| | WGQGTTVTVSS | | | |

TABLE 18
Variable region sequences of anti-CD16 antibody

| | Amino acid sequences of anti-CD16 antibody variable region | | | |
|---|---|---|---|---|
| Antibody | (Bold and underlined amino acids are CDR regions) | | | |
| code | | SEQ | | SEQ |
| (sequence | | ID | | ID |
| source) | VH | NO | VL | NO |
| NM3E2 | EVQLVESGGGVVRPGGSLRLSCAASGFT | 122 | SELTQDPAVSVALGQTVRITCQGDSLRSYYASWYQQKPGQAPVLVIYG | 123 |
| | FDDYGMSWVRQAPGKGLEWVSGINWNGG | | KNNRPSGIPDRFSGSSSGNTASLTITGAQAEDEADYYCNSRDSSGNHV | |
| | STGYADSVKGRFTISRDNAKNSLYLQMN | | VFGGGTKLTVL | |
| | SLRAEDTAVYYCARGRSLLFDYWGQGTL | | | |
| | VTVSR | | | |

TABLE 19
Variable region sequences of anti-SLAMF7 antibody

| | Amino acid sequences of anti-SLAMF7 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| Elotuzumab | EVQLVESGGGLVQPGGSLRLSCAASGFD | 124 | DIQMTQSPSSLSASVGDRVTITCKASQDVGIAVAWYQQKPGKVP | 125 |
| (WO2004100898A2) | FSRYWMSWVRQAPGKGLEWIGEINPDSS | | KLLIYWASTRHTGVPDRFSGSGSGTDFTLTISSLQPEDVATYYC | |
| | TINYAPSLKDKFIISRDNAKNSLYLQMN | | QQYSSYPYTFGQGTKVEIK | |
| | SLRAEDTAVYYCARPDGNYWYFDVWGQG | | | |
| | TLVTVSS | | | |

TABLE 20
Variable region sequences of anti-CEA antibody

| | Amino acid sequences of anti-CEA antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| hPR1A3(Cancer | QVQLVQSGSELKKPGASVKVSCKASGYT | 126 | DIQMTQSPSSLSASVGDRVTITCKASQNVGTNVAWYQQKPGKAPKLLI | 127 |
| Immunol | FTVFGMNWVRQAPGQGLEWMGWINTKTG | | YSASYRYSGVPSRFSGSGSGTDFTFTISSLQPEDIATYYCHQYYTYPL | |
| Immunother | EATYVEEFKGRFVFSLDTSVSTAYLQIS | | FTFGQGTKVEIK | |
| (1999) 47: | SLKADDTAVYYCARWDFYDYVEAMDYWG | | | |
| 299-306) | QGTTVTVSS | | | |

TABLE 21
Variable region sequences of anti-VEGF antibody

| | Amino acid sequences of anti-VEGF antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| Avastin | EVQLVESGGGLVQPGGSLRLSCAASGYT | 128 | DIQMTQSPSSLSASVGDRVTITCSASQDISNYLNWYQQKPGKAP | 129 |
| | FTNYGMNWVRQAPGKGLEWVGWINTYTG | | KVLIYFTSSLHSGVPSRFSGSGSGTDFTLTISSLQPEDFATYYC | |
| | EPTYAADFKRRFTFSLDTSKSTAYLQMN | | QQYSTVPWTFGQGTKVEIK | |
| | SLRAEDTAVYYCAKYPHYYGSSHWYFDV | | | |
| | WGQGTLVTVSS | | | |
| B2041 | EVQLVESGGGLVQPGGSLRLSCAASGFS | 130 | DIQMTQSPSSLSASVGDRVTITCRASQVIRRSLAWYQQKPGKAP | 131 |
| (WO2005012359A2) | INGSWIFWVRQAPGKGLEWVGAIWPFGG | | KLLIYAASNLASGVPSRFSGSGSGTDFTLTISSLQPEDFATYYC | |
| | YTHYADSVKGRFTISADTSKNTAYLQMN | | QQSNTSPLTFGQGTKVEIK | |
| | SLRAEDTAVYYCARWGHSTSPWAMDYWG | | | |
| | QGTLVTVSS | | | |
| G631 | EVQLVESGGGLVQPGGSLRLSCAASGFT | 132 | DIQMTQSPSSLSASVGDRVTITCRASQDVSTAVAWYQQKPGKAP | 133 |
| (WO2005012359A2) | ISDYWIHWVRQAPGKGLEWVAGITPAGG | | KLLIYSASFLYSGVPSRFSGSGSGTDFTLTISSLQPEDFATYYC | |
| | YTYYADSVKGRFTISADTSKNTAYLQMN | | QQGYGNPFTFGQGTKVEIK | |
| | SLRAEDTAVYYCARFVFFLPYAMDYWGQ | | | |
| | GTLVTVSS | | | |

TABLE 22
Anti-TGF-beta antibody variable regions

| | Amino acid sequences of anti-TGF-beta antibody variable region | | | |
|---|---|---|---|---|
| Antibody | (Bold and underlined amino acids are CDR regions) | | | |
| code | | SEQ | | SEQ |
| (sequence | | ID | | ID |
| source) | VH | NO | VL | NO |
| 3G12 | QVQLVQSGAEVKKPGSSVKVSCKASGYT | 134 | ETVLTQSPGTLSLSPGERATLSCRASQSLGSSYLAWYQQKPGQAPRLL | 135 |
| | FSSNVISWVRQAPGQGLEWMGGVIPIVD | | IYGASSRAPGIPDRFSGSGSGTDFTLTISRLEPEDFAVYYCQQYADSP | |
| | IANYAQRFKGRVTITADESTSTTYMELS | | ITFGQGTRLEIK | |
| | SLRSEDTAVYYCASTLGLVLDAMDYWGQ | | | |
| | GTLVTVSS | | | |
| 4B9 | QVQLVQSGAEVKKPGSSVKVSCKASGYT | 136 | ETVLTQSPGTLSLSPGERATLSCRASQSLGSSYLAWYQQKPGQAPRLL | 137 |
| | FSSNVISWVRQAPGQGLEWMGGVIPIVD | | IYGASSRAPGIPDRFSGSGSGTDFTLTISRLEPEDFAVYYCQQYADSP | |
| | IANYAQRFKGRVTITADESTSTTYMELS | | ITFGQGTRLEIK | |
| | SLRSEDTAVYYCALPRAFVLDAMDYWGQ | | | |
| | GTLVTVSS | | | |

TABLE 23
Anti-IL-10 antibody variable regions

| | Amino acid sequences of anti-IL-10 antibody variable region | | | |
|---|---|---|---|---|
| Antibody | (Bold and underlined amino acids are CDR regions) | | | |
| code | | SEQ | | SEQ |
| (sequence | | ID | | ID |
| source) | VH | NO | VL | NO |
| B-N10 | QVQLKQSGPGLLQPSQSLSISCTVSGFS | 138 | DVLMTQTPLSLPVSLGDQASISCRSSQNIVHSNGNTYLEWYLQKPGQS | 139 |
| | LATYGVHWVRQSPGKGLEWLGVIWRGGS | | PKLLIYKVSNRFSGVPDRFSGSGSGTDFTLKITRLEAEDLGVYYCFQG | |
| | TDYSAAFMSRLSITKDNSKSQVFFKMNS | | SHVPWTFGGGTKLEIK | |
| | LQADDTAIYFCAKQAYGHYMDYWGQGTS | | | |
| | VTVSS | | | |
| BT-063 | EVQLVESGGGLVQPGGSLRLSCAASGFS | 140 | DVVMTQSPLSLPVTLGQPASISCRSSQNIVHSNGNTYLEWYLQRPGQS | 141 |
| | FATYGVHWVRQSPGKGLEWLGVIWRGGS | | PRLLIYKVSNRFSGVPDRFSGSGSGTDFTLKISRVEAEDVGVYYCFQG | |
| | TDYSAAFMSRLTISKDNSKNTVYLQMNS | | SHVPWTFGQGTKVEIK | |
| | LRAEDTAVYFCAKQAYGHYMDYWGQGTS | | | |
| | VTVSS | | | |

TABLE 24
Variable region sequences of anti-CD20 antibody

| | Amino acid sequences of anti-CD20 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| Gazyva | QVQLVQSGAEVKKPGSSVKVSCKASGYA | 142 | DIVMTQTPLSLPVTPGEPASISCRSSKSLLHSNGITYLYWYLQKPGQS | 143 |
| (WO2005044859) | FSYSWINWVRQAPGQGLEWMGRIFPGDG | | PQLLIYQMSNLVSGVPDRFSGSGSGTDFTLKISRVEAEDVGVYYCAQN | |
| | DTDYNGKFKGRVTITADKSTSTAYMELS | | LELPYTFGGGTKVEIK | |
| | SLRSEDTAVYYCARNVFDGYWLVYWGQG | | | |
| | TLVTVSS | | | |

TABLE 25
Variable region sequences of anti-Claudinl8.2 antibody

| | Amino acid sequences of anti-Claudinl8.2 antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| IMAB362 | QVQLKQSGPGLLQPSQSLSISCTVSGFS | 144 | DIVMTQSPSSLTVTAGEKVTMSCKSSQSLLNSGNQKNYLTWYQQ | 145 |
| (US20090169547A1) | LATYGVHWVRQSPGKGLEWLGVIWRGGS | | KPGQPPKLLIYWASTRESGVPDRFTGSGSGTDFTLTISSVQAED | |
| | TDYSAAFMSRLSITKDNSKSQVFFKMNS | | LAVYYCQNDYSYPFTFGSGTKLEIK | |
| | LQADDTAIYFCAKQAYGHYMDYWGQGTS | | | |
| | VTVSS | | | |

TABLE 26
Variable region sequences of anti-FIXa antibody

| | Amino acid sequences of anti-FIXa antibody variable region | | | |
|---|---|---|---|---|
| | (Bold and underlined amino acids are CDR region) | | | |
| Antibody code | | SEQ | | SEQ |
| (sequence | | ID | | ID |
| source) | VH | NO | VL | NO |
| A44 | QVQLQQSGAELAKPGASVKLSCKASGYT | 146 | DIVMTQSHKFMSTSVGDRVSITCKASQDVGTAVAWYQQKPGQSPKLLI | 147 |
| (US8062635B2) | FTSSWMHWIKQRPGQGLEWLGYINPSSG | | YWASTRHTGVPDRFTGSRYGTDFTLTISNVQSEDLADYLCQQYSNYIT | |
| | YTKYNRKFRDKATLTADKSSSTAYMQLT | | FGGGTKLELK | |
| | SLTYEDSAVYYCARGGNGYYFDYWGQGT | | | |
| | TLTVSS | | | |
| A50 | QVQLQQSGAELAKPGASVKLSCKASGYT | 148 | DIVMTQSHKFMSTSVGDRVSITCKASQDVGTAVAWYQQKPGLSPKLLI | 149 |
| (US8062635B2) | FTTYWMHWVKQRPGQGLEWIGYINPSSG | | YWASTRHTGVPDRFTGSGSGTDFTLTISNVQSEDLADYFCQQYSSYLT | |
| | YTKYNQKFKVKATLTADKSSSTAYMQLS | | FGAGTKLEIK | |
| | SLTDEDSAVYYCANGNLGYFFDYWGQGT | | | |
| | TLTVSS | | | |
| A69 | EVQLQQSGAELVKPGASVKLSCTASGFN | 150 | DIQMTQSHKFMSTSVGDRVSITCKASQDVSTAVAWYQQKPGQSPKLLI | 151 |
| (US8062635B2) | IKDYYMHWIKQRPGQGLEWLGYINPSSG | | YWASTRHTGVPDRFTGSGSGTDFTLTISNVQSEDLADYLCQQYSNYIT | |
| | YTKYNRKFRDKATLTADKSSSTAYMQLT | | FGAGTKLELK | |
| | SLTYEDSAVYYCARGGNGYYLDYWGQGT | | | |
| | TLTVSS | | | |
| XB12 | EVQLQQSGPGLVKPTQSLSLTCSVTGYS | 152 | DIVLTQSPAIMSASLGEKVTMSCRATSSVNYIYWYQQKSDASPKLWIF | 153 |
| (US8062635B2) | ITSGYYWTWIRQFPGNNLEWIGYISFDG | | YTSNLAPGVPPRFSGSGSGNSYSLTISSMEAEDAATYYCQQFSSSPWT | |
| | TNDYNPSLKNRISITRDTSENQFFLKLN | | FGGGTKLEIK | |
| | SVTTEDTATYYCARGPPCTYWGQGTLVT | | | |
| | VSA | | | |

TABLE 27
Variable region sequences of anti-FX antibody

| | Amino acid sequences of anti-FX antibody variable region | | | |
|---|---|---|---|---|
| | (Bold and underlined amino acids are CDR regions) | | | |
| Antibody code | | SEQ | | SEQ |
| (sequence | | ID | | ID |
| source) | VH | NO | VL | NO |
| SB04 | QVQLQQSGPELVKPGASVKMSCKASGYT | 154 | DIVMTQSPSSLAVSVGEKVTMSCKSSQSLLYSSNQKNYLAWYQQ | 155 |
| (US8062635B2) | FTHFVLHWVKQNPGQGLEWIGYIIPYND | | KPGQSPKLLIYWASTRESGVPDRFTGSGSGTDFTLTISSVKAED | |
| | GTKYNEKFKGKATLTSDKSSSTAYMELS | | LAVYLCQQYYRFPYTFGGGTKLEIK | |
| | SLTSEDSAVYYCARGNRYDVGSYAMDYW | | | |
| | GQGTSVTVSS | | | |
| B26 | QVQLQQSGPELVKPGASVKISCKASGYT | 156 | DIVLTQSQKFMSTSVGDRVSITCKASQNVGTAVAWYQQKPGQSP | 157 |
| (US8062635B2) | FTDNNMDWVKQSHGKGLEWIGDINTKSG | | KALIYSASYRYSGVPDRFTGSGSGTDFTLTISNVQSEDLAEYFC | |
| | GSIYNQKFKGKATLTIDKSSSTAYMELR | | QQYNSYPLTFGAGTKLEIK | |
| | SLTSEDTAVYYCARRRSYGYYFDYWGQG | | | |
| | TTLTVSS | | | |

TABLE 28
Variable region sequences of anti-HER2 antibody

| | Amino acid sequences of anti-HER2 antibody variable region | | | |
|---|---|---|---|---|
| Antibody | (Bold and underlined amino acids are CDR regions) | | | |
| code | | SEQ | | SEQ |
| (sequence | | ID | | ID |
| source) | VH | NO | VL | NO |
| Herceptin | EVQLVESGGGLVQPGGSLRLSCAASGFN | 158 | DIQMTQSPSSLSASVGDRVTITCRASQDVNTAVAWYQQKPGKAPKLLI | 159 |
| | IKDTYIHWVRQAPGKGLEWVARIYPTNG | | YSASFLYSGVPSRFSGSRSGTDFTLTISSLQPEDFATYYCQQHYTTPP | |
| | YTRYADSVKGRFTISADTSKNTAYLQMN | | TFGQGTKVEIK | |
| | SLRAEDTAVYYCSRWGGDGFYAMDYWGQ | | | |
| | GTLVTVSS | | | |
| Perjeta | EVQLVESGGGLVQPGGSLRLSCAASGFT | 160 | DIQMTQSPSSLSASVGDRVTITCKASQDVSIGVAWYQQKPGKAPKLLI | 161 |
| | FTDYTMDWVRQAPGKGLEWVADVNPNSG | | YSASYRYTGVPSRFSGSGSGTDFTLTISSLQPEDFATYYCQQYYIYPY | |
| | GSIYNQRFKGRFTLSVDRSKNTLYLQMN | | TFGQGTKVEIK | |
| | SLRAEDTAVYYCARNLGPSFYFDYWGQG | | | |
| | TLVTVSS | | | |

TABLE 29
Variable region sequences of anti-Siglec-15 antibody

| | Amino acid sequences of anti-Siglec-15 antibody variable region | | | |
|---|---|---|---|---|
| Antibody | Bold and underlined amino acids are CDR regions) | | | |
| code | VH | SEQ | VL | SEQ |
| (sequence | | ID | | ID |
| source) | | NO | | NO |
| 34A1 | EVQILETGGGLVKPGGSLRLSCATSGFN | 162 | DIVLTQSPALAVSLGQRATISCRASQSVTISGYSFIHWYQQKPGQQPR | 163 |
| | FNDYFMNWVRQAPEKGLEWVAQIRNKIY | | LLIYRASNLASGIPARFSGSGSGTDFTLTINPVQADDIATYFCQQSRK | |
| | TYATFYAESLEGRVTISRDDSESSVYLQ | | SPWTFAGGTKLELR | |
| | VSSLRAEDTAIYYCTRSLTGGDYFDYWG | | | |
| | QGVMVTVSS | | | |
| H34A1 | EVQLVESGGGLVQPGGSLRLSCAASGFN | 164 | EILMTQSPATLSLSPGERATLSCRASQSVTISGYSFIHWYQQKPGQAP | 165 |
| | FNDYFMNWVRQAPGKGLEWVAQIRNKIY | | RLLIYRASNLASGIPARFSGSGSGTDFTLTISSLEPEDFALYYCQQSR | |
| | TYATFYAASVKGRFTISRDNAKNSLYLQ | | KSPWTFGQGTKVEIK | |
| | MNSLRAEDTAVYYCARSLTGGDYFDYWG | | | |
| | QGTLVTVSS | | | |

TABLE 30
Variable region sequences of anti-luciferase antibody

| | Amino acid sequences of anti-luciferase antibody variable region | | | |
|---|---|---|---|---|
| Antibody | (Bold and underlined amino acids are CDR regions) | | | |
| code | | SEQ | | SEQ |
| (sequence | | ID | | ID |
| source) | VH | NO | VL | NO |
| 4420 | EVKLDETGGGLVQPGRPMKLSCVASGFT | 166 | DWMTQTPLSLPVSLGDQASISCRSSQSLVHSNGNTYLRWYLQKPGQS | 167 |
| | FSDYWMNWVRQSPEKGLEWVAQIRNKPY | | PKVLIYKVSNRFSGVPDRFSGSGSGTDFTLKISRVEAEDLGVYFCSQS | |
| | NYETYYSDSVKGRFTISRDDSKSSVYLQ | | THVPWTFGGGTKLEIK | |
| | MNNLRVEDMGIYYCTGSYYGMDYWGQGT | | | |
| | SVTVSS | | | |

TABLE 31
Amino acid sequences of some components A

| | | Expression | | Corresponding | SEQ |
|---|---|---|---|---|---|
| Code | Peptide | plasmid name | Domain | code | ID NO |
| A-Fab10 | A-HIn | pA-HIn(10) | VHa | S70 | 118 |
| | | | CH1 | G1CH1 | 179 |
| | | | flanking sequence a | FSa1 | 51 |
| | | | In | SspDnaE | 31 |
| | | | tag protein | His-tag | 24 |
| | | | | Strep-tag | 28 |
| | A-L | pA-L | VLa | S70 | 119 |
| | | | CL | Lc1 | 172 |
| A-Fab11 | A-HIn | pA-HIn(11) | VHa | S70 | 118 |
| | | | CH1 | G1CH1 | 179 |
| | | | flanking sequence a | FSa10 | 60 |
| | | | In | SspDnaE | 31 |
| | | | tag protein | His-tag | 24 |
| | | | | Strep-tag | 28 |
| | A-L | pA-L | VLa | S70 | 119 |
| | | | CL | Lc1 | 172 |
| A-Fab20 | A-HIn | pA-HIn(20) | VHa | S70 | 118 |
| | | | CH1 | G1CH1 | 179 |
| | | | flanking sequence a | FSa2 | 52 |
| | | | In | SspDnaB | 33 |
| | | | tag protein | His-tag | 24 |
| | | | | Strep-tag | 28 |
| | A-L | pA-L | VLa | S70 | 119 |
| | | | CL | Lc1 | 172 |
| A-Fab21 | A-HIn | pA-HIn(21) | VHa | S70 | 118 |
| | | | CH1 | G1CH1 | 179 |
| | | | flanking sequence a | FSa10 | 60 |
| | | | In | SspDnaB | 33 |
| | | | tag protein | His-tag | 24 |
| | | | | Strep-tag | 28 |
| | A-L | pA-L | VLa | S70 | 119 |
| | | | CL | Lc1 | 172 |
| A-Fab30 | A-HIn | pA-HIn(30) | VHa | S70 | 118 |
| | | | CH1 | G1CH1 | 179 |
| | | | flanking sequence a | FSa5 | 55 |
| | | | In | MxeGyrA | 35 |
| | | | tag protein | His-tag | 24 |
| | | | | Strep-tag | 28 |
| | A-L | pA-L | VLa | S70 | 119 |
| | | | CL | Lc1 | 172 |
| A-Fab31 | A-HIn | pA-HIn(31) | VHa | S70 | 118 |
| | | | CH1 | G1CH1 | 179 |
| | | | flanking sequence a | FSa10 | 60 |
| | | | In | MxeGyrA | 35 |
| | | | tag protein | His-tag | 24 |
| | | | | Strep-tag | 28 |
| | A-L | pA-L | VLa | S70 | 119 |
| | | | CL | Lc1 | 172 |
| A-Fab40 | A-HIn | pA-HIn(40) | VHa | S70 | 118 |
| | | | CH1 | G1CH1 | 179 |
| | | | flanking sequence a | FSa6 | 56 |
| | | | In | MjaTFIIB | 37 |
| | | | tag protein | His-tag | 24 |
| | | | | Strep-tag | 28 |
| | A-L | pA-L | VLa | S70 | 119 |
| | | | CL | Lc1 | 172 |
| A-Fab41 | A-HIn | pA-HIn(41) | VHa | S70 | 118 |
| | | | CH1 | G1CH1 | 179 |
| | | | flanking sequence a | FSa10 | 60 |
| | | | In | MjaTFIIB | 37 |
| | | | tag protein | His-tag | 24 |
| | | | | Strep-tag | 28 |
| | A-L | pA-L | VLa | S70 | 119 |
| | | | CL | Lc1 | 172 |
| A-Fab50 | A-HIn | pA-HIn(50) | VHa | S70 | 118 |
| | | | CH1 | G1CH1 | 179 |
| | | | flanking sequence a | FSa7 | 57 |
| | | | In | PhoVMA | 39 |
| | | | tag protein | His-tag | 24 |
| | | | | Strep-tag | 28 |
| | A-L | pA-L | VLa | S70 | 119 |
| | | | CL | Lc1 | 172 |
| A-Fab51 | A-HIn | pA-HIn(51) | VHa | S70 | 118 |
| | | | CH1 | G1CH1 | 179 |
| | | | flanking sequence a | FSa10 | 60 |
| | | | In | PhoVMA | 39 |
| | | | tag protein | His-tag | 24 |
| | | | | Strep-tag | 28 |
| | A-L | pA-L | VLa | S70 | 119 |
| | | | CL | Lc1 | 172 |
| A-Fab60 | A-HIn | pA-HIn(60) | VHa | S70 | 118 |
| | | | CH1 | G1CH1 | 179 |
| | | | flanking sequence a | FSa7 | 57 |
| | | | In | TvoVMA | 41 |
| | | | tag protein | His-tag | 24 |
| | | | | Strep-tag | 28 |
| | A-L | pA-L | VLa | S70 | 119 |
| | | | CL | Lc1 | 172 |
| A-Fab61 | A-HIn | pA-HIn(61) | VHa | S70 | 118 |
| | | | CH1 | G1CH1 | 179 |
| | | | flanking sequence a | FSa10 | 60 |
| | | | In | TvoVMA | 41 |
| | | | tag protein | His-tag | 24 |
| | | | | Strep-tag | 28 |
| | A-L | pA-L | VLa | S70 | 119 |
| | | | CL | Lc1 | 172 |
| A-Fab70 | A-HIn | pA-HIn(70) | VHa | S70 | 118 |
| | | | CH1 | G1CH1 | 179 |
| | | | flanking sequence a | FSa11 | 61 |
| | | | In | Gp41-1 | 43 |
| | | | tag protein | His-tag | 24 |
| | | | | Strep-tag | 28 |
| | A-L | pA-L | VLa | S70 | 119 |
| | | | CL | Lc1 | 172 |
| A-Fab71 | A-HIn | pA-HIn(71) | VHa | S70 | 118 |
| | | | CH1 | G1CH1 | 179 |
| | | | flanking sequence a | FSa10 | 60 |
| | | | In | Gp41-1 | 43 |
| | | | tag protein | His-tag | 24 |
| | | | | Strep-tag | 28 |
| | A-L | pA-L | VLa | S70 | 119 |
| | | | CL | Lc1 | 172 |
| A-Fab80 | A-HIn | pA-HIn(80) | VHa | S70 | 118 |
| | | | CH1 | G1CH1 | 179 |
| | | | flanking sequence a | FSa8 | 58 |
| | | | In | Gp41-8 | 45 |
| | | | tag protein | His-tag | 24 |
| | | | | Strep-tag | 28 |
| | A-L | pA-L | VLa | S70 | 119 |
| | | | CL | Lc1 | 172 |
| A-Fab81 | A-HIn | pA-HIn(81) | VHa | S70 | 118 |
| | | | CH1 | G1CH1 | 179 |
| | | | flanking sequence a | FSa10 | 60 |
| | | | In | Gp41-8 | 45 |
| | | | tag protein | His-tag | 24 |
| | | | | Strep-tag | 28 |
| | A-L | pA-L | VLa | S70 | 119 |
| | | | CL | Lc1 | 172 |
| A-Fab90 | A-HIn | pA-HIn(90) | VHa | S70 | 118 |
| | | | CH1 | G1CH1 | 179 |
| | | | flanking sequence a | FSa9 | 59 |
| | | | In | IMPDH-1 | 47 |
| | | | tag protein | His-tag | 24 |
| | | | | Strep-tag | 28 |
| | A-L | pA-L | VLa | S70 | 119 |
| | | | CL | Lc1 | 172 |
| A-Fab91 | A-HIn | pA-HIn(91) | VHa | S70 | 118 |
| | | | CH1 | G1CH1 | 179 |
| | | | flanking sequence a | FSa10 | 60 |
| | | | In | IMPDH-1 | 47 |
| | | | tag protein | His-tag | 24 |
| | | | | Strep-tag | 28 |
| | A-L | pA-L | VLa | S70 | 119 |
| | | | CL | Lc1 | 172 |
| A-Fab92 | A-HIn | pA-HIn(92) | VHa | S70 | 118 |
| | | | CH1 | G1CH1 | 179 |
| | | | flanking sequence a | FSa14 | 202 |
| | | | In | IMPDH-1 | 47 |
| | | | tag protein | His-tag | 24 |
| | | | | Strep-tag | 28 |
| | A-L | pA-L | VLa | S70 | 119 |
| | | | CL | Lc1 | 172 |
| A-Fab100 | A-HIn | pA-HIn(100) | VHa | S70 | 118 |
| | | | CH1 | G1CH1 | 179 |
| | | | flanking sequence a | FSa7 | 57 |
| | | | In | PhoRadA | 49 |
| | | | tag protein | His-tag | 24 |
| | | | | Strep-tag | 28 |
| | A-L | pA-L | VLa | S70 | 119 |
| | | | CL | Lc1 | 172 |
| A-Fab101 | A-HIn | pA-HIn(101) | VHa | S70 | 118 |
| | | | CH1 | G1CH1 | 179 |
| | | | flanking sequence a | FSa10 | 60 |
| | | | In | PhoRadA | 49 |
| | | | tag protein | His-tag | 24 |
| | | | | Strep-tag | 28 |
| | A-L | pA-L | VLa | S70 | 119 |
| | | | CL | Lc1 | 172 |

Note:
The sequences of domains such as VHa, CH1, flanking sequence a, tag protein, VLa and CL in the table can be replaced with the protein sequences of other corresponding domains mentioned in the present specification.

TABLE 32
Amino acid sequences of some components B including different inteins

| | | Expression | | Corresponding | SEQ |
|---|---|---|---|---|---|
| Code | Peptide | plasmid name | Domain | code | ID NO |
| B-FcIc10 | component B | pTag-Ic-FSb-(B-FcIc10) | tag protein | Strep-tag | 28 |
| | | | | His-tag | 24 |
| | | | Ic | SspDnaE | 32 |
| | | | flanking sequence b | FSb1 | 64 |
| | | | Pb | G1CH2 | 183 |
| | | | | G1CH3 | 188 |
| B-FcIc11 | component B | pTag-Ic-FSb-(B-FcIc11) | tag protein | Strep-tag | 28 |
| | | | | His-tag | 24 |
| | | | Ic | SspDnaE | 32 |
| | | | flanking sequence b | FSb14 | 77 |
| | | | Pb | G1CH2 | 183 |
| | | | | G1CH3 | 188 |
| B-FcIc20 | component B | pTag-Ic-FSb-(B-FcIc20) | tag protein | Strep-tag | 28 |
| | | | | His-tag | 24 |
| | | | Ic | SspDnaB | 34 |
| | | | flanking sequence b | FSb3 | 64 |
| | | | Pb | G1CH2 | 183 |
| | | | | G1CH3 | 188 |
| B-FcIc21 | component B | pTag-Ic-FSb-(B-FcIc21) | tag protein | Strep-tag | 28 |
| | | | | His-tag | 24 |
| | | | Ic | SspDnaB | 34 |
| | | | flanking sequence b | FSb14 | 77 |
| | | | Pb | G1CH2 | 183 |
| | | | | G1CH3 | 188 |
| B-FcIc30 | component B | pTag-Ic-FSb-(B-FcIc30) | tag protein | Strep-tag | 28 |
| | | | | His-tag | 24 |
| | | | Ic | MxeGyrA | 36 |
| | | | flanking sequence b | FSb4 | 67 |
| | | | Pb | G1CH2 | 183 |
| | | | | G1CH3 | 188 |
| B-FcIc31 | component B | pTag-Ic-FSb-(B-FcIc31) | tag protein | Strep-tag | 28 |
| | | | | His-tag | 24 |
| | | | Ic | MxeGyrA | 36 |
| | | | flanking sequence b | FSb14 | 77 |
| | | | Pb | G1CH2 | 183 |
| | | | | G1CH3 | 188 |
| B-FcIc40 | component B | pTag-Ic-FSb-(B-FcIc40) | tag protein | Strep-tag | 28 |
| | | | | His-tag | 24 |
| | | | Ic | MjaTFIIB | 38 |
| | | | flanking sequence b | FSb5 | 68 |
| | | | Pb | G1CH2 | 183 |
| | | | | G1CH3 | 188 |
| B-FcIc41 | component B | pTag-Ic-FSb-(B-FcIc41) | tag protein | Strep-tag | 28 |
| | | | | His-tag | 24 |
| | | | Ic | MjaTFIIB | 38 |
| | | | flanking sequence b | FSb14 | 77 |
| | | | Pb | G1CH2 | 183 |
| | | | | G1CH3 | 188 |
| B-FcIc50 | component B | pTag-Ic-FSb-(B-FcIc50) | tag protein | Strep-tag | 28 |
| | | | | His-tag | 24 |
| | | | Ic | PhoVMA | 40 |
| | | | flanking sequence b | FSb6 | 69 |
| | | | Pb | G1CH2 | 183 |
| | | | | G1CH3 | 188 |
| B-FcIc51 | component B | pTag-Ic-FSb-(B-FcIc51) | tag protein | Strep-tag | 28 |
| | | | | His-tag | 24 |
| | | | Ic | PhoVMA | 40 |
| | | | flanking sequence b | FSb14 | 77 |
| | | | Pb | G1CH2 | 183 |
| | | | | G1CH3 | 188 |
| B-FcIc60 | component B | pTag-Ic-FSb-(B-FcIc60) | tag protein | Strep-tag | 28 |
| | | | | His-tag | 24 |
| | | | Ic | TvoVMA | 42 |
| | | | flanking sequence b | FS6 | 69 |
| | | | Pb | G1CH2 | 183 |
| | | | | G1CH3 | 188 |
| B-FcIc61 | component B | pTag-Ic-FSb-(B-FcIc61) | tag protein | Strep-tag | 28 |
| | | | | His-tag | 24 |
| | | | Ic | TvoVMA | 42 |
| | | | flanking sequence b | FSb14 | 77 |
| | | | Pb | G1CH2 | 183 |
| | | | | G1CH3 | 188 |
| B-FcIc70 | component B | pTag-Ic-FSb-(B-FcIc70) | tag protein | Strep-tag | 28 |
| | | | | His-tag | 24 |
| | | | Ic | Gp41-1 | 44 |
| | | | flanking sequence b | FSb7 | 70 |
| | | | Pb | G1CH2 | 183 |
| | | | | G1CH3 | 188 |
| B-FcIc71 | component B | pTag-Ic-FSb-(B-FcIc71) | tag protein | Strep-tag | 28 |
| | | | | His-tag | 24 |
| | | | Ic | Gp41-1 | 44 |
| | | | flanking sequence b | FSb14 | 77 |
| | | | Pb | G1CH2 | 183 |
| | | | | G1CH3 | 188 |
| B-FcIc80 | component B | pTag-Ic-FSb-(B-FcIc80) | tag protein | Strep-tag | 28 |
| | | | | His-tag | 24 |
| | | | Ic | Gp41-8 | 46 |
| | | | flanking sequence b | FSb8 | 71 |
| | | | Pb | G1CH2 | 183 |
| | | | | G1CH3 | 188 |
| B-FcIc81 | component B | pTag-Ic-FSb-(B-FcIc81) | tag protein | Strep-tag | 28 |
| | | | | His-tag | 24 |
| | | | Ic | Gp41-8 | 46 |
| | | | flanking sequence b | FSb14 | 77 |
| | | | Pb | G1CH2 | 183 |
| | | | | G1CH3 | 188 |
| B-FcIc90 | component B | pTag-Ic-FSb-(B-FcIc90) | tag protein | Strep-tag | 28 |
| | | | | His-tag | 24 |
| | | | Ic | IMPDH-1 | 48 |
| | | | flanking sequence b | FSb9 | 72 |
| | | | Pb | G1CH2 | 183 |
| | | | | G1CH3 | 188 |
| B-FcIc91 | component B | pTag-Ic-FSb-(B-FcIc91) | tag protein | Strep-tag | 28 |
| | | | | His-tag | 24 |
| | | | Ic | IMPDH-1 | 48 |
| | | | flanking sequence b | FSb14 | 77 |
| | | | Pb | G1CH2 | 183 |
| | | | | G1CH3 | 188 |
| B-FcIc92 | component B | pTag-Ic-FSb-(B-FcIc92) | tag protein | Strep-tag | 28 |
| | | | | His-tag | 24 |
| | | | Ic | IMPDH-1 | 48 |
| | | | flanking sequence b | FSb17 | 204 |
| | | | Pb | G1CH2 | 183 |
| | | | | G1CH3 | 188 |
| B-FcIc100 | component B | pTag-Ic-FSb-(B-FcIc100) | tag protein | Strep-tag | 28 |
| | | | | His-tag | 24 |
| | | | Ic | PhoRadA | 50 |
| | | | flanking sequence b | FSb10 | 73 |
| | | | Pb | G1CH2 | 183 |
| | | | | G1CH3 | 188 |
| B-FcIc101 | component B | pTag-Ic-FSb-(B-FcIc101) | tag protein | Strep-tag | 28 |
| | | | | His-tag | 24 |
| | | | Ic | PhoRadA | 50 |
| | | | flanking sequence b | FSb14 | 77 |
| | | | Pb | G1CH2 | 183 |
| | | | | G1CH3 | 188 |

TABLE 33
Component B′ including different inteins

| | | | | | |
|---|---|---|---|---|---|
| | | Expression | | Corresponding | SEQ |
| Code | Polypeptide | plasmid name | Domain | code | ID NO |

Types of inteins SspDnaE

| | | | | | |
|---|---|---|---|---|---|
| B′-HAb10 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(10) | tag protein | Strep-tag | 28 |
| | | | | His-tag | 24 |
| | | | Ic | SspDnaE | 32 |
| | | | flanking sequence b | FSb1 | 64 |
| | | | CH2 | G1CH2 | 183 |
| | | | CH3 | G1CH3 | 188 |
| B′-HAb11 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(11) | tag protein | Strep-tag | 28 |
| | | | | His-tag | 24 |
| | | | Ic | SspDnaE | 32 |
| | | | flanking sequence b | FSb14 | 77 |
| | | | CH2 | G1CH2 | 183 |
| | | | CH3 | G1CH3 | 188 |
| B′-HAb20 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(20) | tag protein | Strep-tag | 28 |
| | | | | His-tag | 24 |
| | | | Ic | SspDnaB | 34 |
| | | | flanking sequence b | FSb3 | 66 |
| | | | CH2 | G1CH2 | 183 |
| | | | CH3 | G1CH3 | 188 |
| B′-HAb21 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(21) | tag protein | Strep-tag | 28 |
| | | | | His-tag | 24 |
| | | | Ic | SspDnaB | 34 |
| | | | flanking sequence b | FSb14 | 77 |
| | | | CH2 | G1CH2 | 183 |
| | | | CH3 | G1CH3 | 188 |

Types of inteins MxeGyrA

| | | | | | |
|---|---|---|---|---|---|
| B′-HAb30 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(30) | tag protein | Strep-tag | 28 |
| | | | | His-tag | 24 |
| | | | Ic | MxeGyrA | 36 |
| | | | flanking sequence b | FSb4 | 67 |
| | | | CH2 | G1CH2 | 183 |
| | | | CH3 | G1CH3 | 188 |
| B′-HAb31 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(31) | tag protein | Strep-tag | 28 |
| | | | | His-tag | 24 |
| | | | Ic | MxeGyrA | 36 |
| | | | flanking sequence b | FSb14 | 77 |
| | | | CH2 | G1CH2 | 183 |
| | | | CH3 | G1CH3 | 188 |

Types of inteins MjaTFIIB

| | | | | | |
|---|---|---|---|---|---|
| B′-HAb40 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(40) | tag protein | Strep-tag | 28 |
| | | | | His-tag | 24 |
| | | | Ic | MjaTFIIB | 38 |
| | | | flanking sequence b | FSb5 | 68 |
| | | | CH2 | G1CH2 | 183 |
| | | | CH3 | G1CH3 | 188 |
| B′-HAb41 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(41) | tag protein | Strep-tag | 28 |
| | | | | His-tag | 24 |
| | | | Ic | MjaTFIIB | 38 |
| | | | flanking sequence b | FSb14 | 77 |
| | | | CH2 | G1CH2 | 183 |
| | | | CH3 | G1CH3 | 188 |

Types of inteins PhoVMA

| | | | | | |
|---|---|---|---|---|---|
| B′-HAb50 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(50) | tag protein | Strep-tag | 28 |
| | | | | His-tag | 24 |
| | | | Ic | PhoVMA | 40 |
| | | | flanking sequence b | FSb6 | 69 |
| | | | CH2 | G1CH2 | 183 |
| | | | CH3 | G1CH3 | 188 |
| B′-HAb51 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(51) | tag protein | Strep-tag | 28 |
| | | | | His-tag | 24 |
| | | | Ic | PhoVMA | 40 |
| | | | flanking sequence b | FSb14 | 77 |
| | | | CH2 | G1CH2 | 183 |
| | | | CH3 | G1CH3 | 188 |

Types of inteins TvoVMA

| | | | | | |
|---|---|---|---|---|---|
| B′-HAb60 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(60) | tag protein | Strep-tag | 28 |
| | | | | His-tag | 24 |
| | | | Ic | TvoVMA | 42 |
| | | | flanking sequence b | FS6 | 69 |
| | | | CH2 | G1CH2 | 183 |
| | | | CH3 | G1CH3 | 188 |
| B′-HAb61 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(61) | tag protein | Strep-tag | 28 |
| | | | | His-tag | 24 |
| | | | Ic | TvoVMA | 42 |
| | | | flanking sequence b | FSb14 | 77 |
| | | | CH2 | G1CH2 | 183 |
| | | | CH3 | G1CH3 | 188 |

Types of inteins Gp41-1

| | | | | | |
|---|---|---|---|---|---|
| B′-HAb70 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(70) | tag protein | Strep-tag | 28 |
| | | | | His-tag | 24 |
| | | | Ic | Gp41-1 | 44 |
| | | | flanking sequence b | FSb7 | 70 |
| | | | CH2 | G1CH2 | 183 |
| | | | CH3 | G1CH3 | 188 |
| B′-HAb71 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(71) | tag protein | Strep-tag | 28 |
| | | | | His-tag | 24 |
| | | | Ic | Gp41-1 | 44 |
| | | | flanking sequence b | FSb14 | 77 |
| | | | CH2 | G1CH2 | 183 |
| | | | CH3 | G1CH3 | 188 |

Types of inteins Gp41-8

| | | | | | |
|---|---|---|---|---|---|
| B′-HAb80 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(80) | tag protein | Strep-tag | 28 |
| | | | | His-tag | 24 |
| | | | Ic | Gp41-8 | 46 |
| | | | flanking sequence b | FSb8 | 71 |
| | | | CH2 | G1CH2 | 183 |
| | | | CH3 | G1CH3 | 188 |
| B′-HAb81 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(81) | tag protein | Strep-tag | 28 |
| | | | | His-tag | 24 |
| | | | Ic | Gp41-8 | 46 |
| | | | flanking sequence b | FSb14 | 77 |
| | | | CH2 | G1CH2 | 183 |
| | | | CH3 | G1CH3 | 188 |

Types of inteins IMPDH-1

| | | | | | |
|---|---|---|---|---|---|
| B′-HAb90 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(90) | tag protein | Strep-tag | 28 |
| | | | | His-tag | 24 |
| | | | Ic | IMPDH-1 | 48 |
| | | | flanking sequence b | FSb9 | 72 |
| | | | CH2 | G1CH2 | 183 |
| | | | CH3 | G1CH3 | 188 |
| B′-HAb91 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(91) | tag protein | Strep-tag | 28 |
| | | | | His-tag | 24 |
| | | | Ic | IMPDH-1 | 48 |
| | | | flanking sequence b | FSb14 | 77 |
| | | | CH2 | G1CH2 | 183 |
| | | | CH3 | G1CH3 | 188 |
| B′-HAb92 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(92) | tag protein | Strep-tag | 28 |
| | | | | His-tag | 24 |
| | | | Ic | IMPDH-1 | 48 |
| | | | flanking sequence b | FSb17 | 204 |
| | | | CH2 | G1CH2 | 183 |
| | | | CH3 | G1CH3 | 188 |

Types of inteins PhoRadA

| | | | | | |
|---|---|---|---|---|---|
| B′-HAb100 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(100) | tag protein | Strep-tag | 28 |
| | | | | His-tag | 24 |
| | | | Ic | PhoRadA | 50 |
| | | | flanking sequence b | FSb10 | 73 |
| | | | CH2 | G1CH2 | 183 |
| | | | CH3 | G1CH3 | 188 |
| B′-HAb101 | B′-L | pB′-L | VLb | Dara | 89 |
| | | | CL | Lc1 | 172 |
| | B′-H | pB′-H | VHb | Dara | 88 |
| | | | CH1 | G1CH1 | 179 |
| | | | hinge | Hin1 | 168 |
| | | | CH2 | G1CH2 | 183 |
| | | | CH3 | G1CH3 | 188 |
| | B′-FcIc | pB′-FcIc(101) | tag protein | Strep-tag | 28 |
| | | | | His-tag | 24 |
| | | | Ic | PhoRadA | 50 |
| | | | flanking sequence b | FSb14 | 77 |
| | | | CH2 | G1CH2 | 183 |
| | | | CH3 | G1CH3 | 188 |

Note
The sequences of domains such as VLb, CL, VHb, CH1, hinge, CH2, CH3, and tag protein in the table can be replaced with the protein sequences of other corresponding domains mentioned in the present specification.

### EXAMPLE 1

### Experimental Method

1. Preparation of Recombinant Polypeptides

The DNA sequences in the Examples of the present disclosure were all obtained by reverse translation based on the amino acid sequences, and were synthesized by Wuhan GeneCreate Biological Engineering Co., Ltd.

The recombinant polypeptides involved in the Examples were all prepared by the following method: in the presence of recombinase, the DNA sequence and a vector pcDNA3.1 digested by a restriction enzyme EcoRI were ligated at 37° C. for 30 minutes, and then transformed into a Trans10 competent cell by heat shock method, and then transiently transfected into 293E cells (purchased from Thermo Fisher) after verified by sequencing (Wuhan GeneCreate Biological Engineering Co., Ltd.). After expression, the recombinant polypeptides were purified.

2. The Co-Transfected Plasmids Involved in the Examples were Shown as Follows:

1) To express the component A and component B shown in FIG. 1, the plasmids pPa-FSa-In-Tag and pTag-Ic-FSb-Pb were required to be respectively transfected or co-transfected into 293E cells;

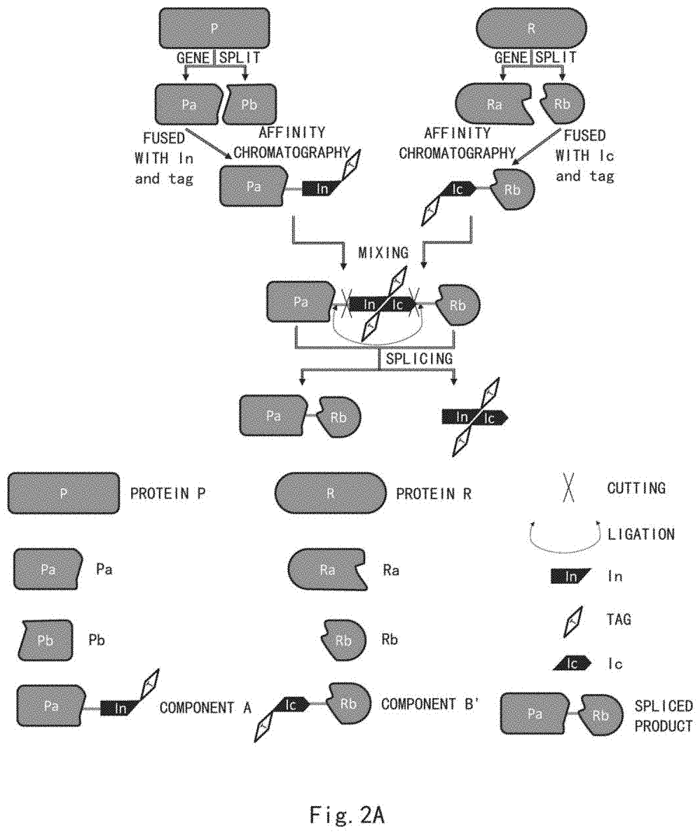

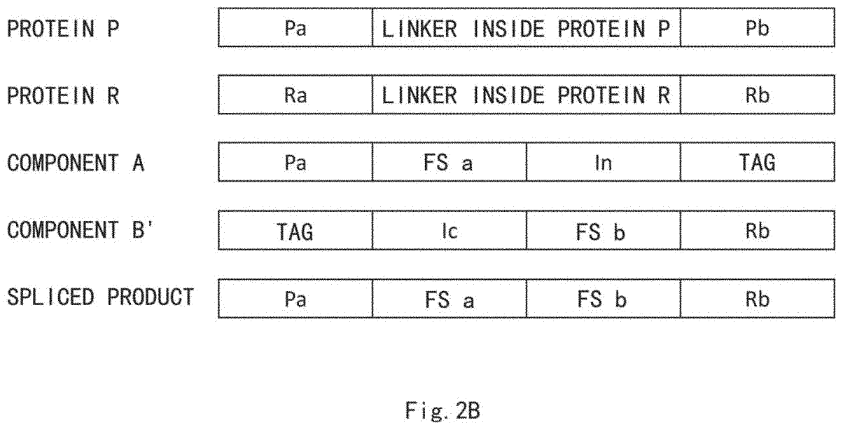

2) To express the component A and component B′ shown in FIG. 2, the plasmids pPa-FSa-In-Tag and pTag-Ic-FSb-Rb were required to be respectively transfected or co-transfected into 293E cells;

3) To express the component A shown in FIG. 3, co-transfection of plasmids Pa-HIn and Pa-L or separate transfection of plasmid pBi-Pa-FSa-In-Tag into 293E cells was required; to express the component B′ shown in FIG. 3, co-transfection of plasmids pB′-L, pB′-H and pB′-FcIc or separate transfection of plasmid pBi-Tag-Ic-FSb-Rb into 293E cells was required.

In general, if two plasmids were co-transfected and expressed, the molar ratio of the two plasmids was 1:1 or any other ratio. If three plasmids were co-transfected and expressed, the molar ratio of the three plasmids was 1:1:1, or any other ratio.

3. Purification of Polypeptides with Tag Proteins

(1) When the tag protein was Fc, the polypeptide was purified by affinity chromatography, for example, MabSelect SuRe (GE, Cat. No. 17-5438-01), 18 ml column.

(2) When the tag protein was His-tag, the polypeptide was purified by affinity chromatography, for example, Ni-NTA (Jiangsu Qianchun, product number: A41002-06).

(3) When the tag protein was Strep-tag, Flag, HA or MBP, etc., the polypeptide was purified by Strep-Tactin affinity chromatography, anti-Flag antibody affinity chromatography, anti-HA antibody affinity chromatography, or cross-linked starch affinity chromatography by selecting corresponding packings and buffers.

(4) When the component A (A′) or component B (B′) did not have a tag protein, the spliced product can be separated by an ion exchange chromatography based on the difference in isoelectric point. The chromatography packing can be a cation exchange chromatography packing or an anion exchange chromatography packing, such as Hitrap SP-HP (GE Company).

(5) When the component A (A′) or component B (B′) did not have a tag protein, the spliced product can be separated by a hydrophobic chromatography based on the difference in hydrophobicity by using a chromatography packing such as Capto phenyl ImpRes packing (GE Company).

(6) When the component A (A′) or component B (B′) did not have a tag protein, the spliced product can be separated by a molecular sieve chromatography based on the difference in molecular weight by using a chromatography packing such as HiLoad Superdex 200pg (GE Company).

### EXAMPLE 2

### Screening of Flanking Sequence Pairs of Inteins such as SspDnaB, MxeGyrA, MjaTFIIB, PhoVMA, TVoVMA, Gp41-1, Gp41-8, IMPDH-1, PhoRadA

Construction of Expression Plasmids A-HIn, pA-L, and Plasmid (pTag-Ic-FSb-Pb)

Under the conditions as described in “Preparation of recombinant polypeptides” of Example 1, as shown in FIGS. 4A and 4B, component expression plasmids for the inteins SspDnaB, MxeGyrA, MjaTFIIB, PhoVMA, TvoVMA, GP41-1, GP41-8, IMPDH-1 and PhoRadA were respectively constructed by pcDNA3.1 plasmid vector based on the structure as shown in Table 31 and Table 32. The pA-L plasmid was the same as that in Example 1.

For the intein SspDnaB, the following plasmids were constructed: plasmids pA-HIn(20)˜pA-HIn(21) corresponding to A-Fab20 and A-Fab21, and plasmids pTag-Ic-FSb-(B-FcIc20) and pTag-Ic-FSb-(B-FcIc21) corresponding to B-FcIc20 and B-FcIc21.

For the intein MxeGyrA, the following plasmids were constructed: plasmids pA-HIn(30)˜pA-HIn(31) corresponding to A-Fab30 and A-Fab31, and plasmids pTag-Ic-FSb-(B-FcIc30) and pTag-Ic-FSb-(B-FcIc31) corresponding to B-FcIc30 and B-FcIc31.

For the intein MjaTFIIB, the following plasmids were constructed: plasmids pA-HIn(40)˜pA-HIn(41) corresponding to A-Fab40 and A-Fab41, and plasmids pTag-Ic-FSb-(B-FcIc40) and pTag-Ic-FSb-(B-FcIc41) corresponding to B-FcIc40 and B-FcIc41.

For the intein PhoVMA, the following plasmids were constructed: plasmids pA-HIn(50)˜pA-HIn(51) corresponding to A-Fab50 and A-Fab51, and plasmids pTag-Ic-FSb-(B-FcIc50) and pTag-Ic-FSb-(B-FcIc51) corresponding to B-FcIc50 and B-FcIc51.

For the intein TVoVMA, the following plasmids were constructed: plasmids pA-HIn(60)˜pA-HIn(61) corresponding to A-Fab60 and A-Fab61, and plasmids pTag-Ic-FSb-(B-FcIc60) and pTag-Ic-FSb-(B-FcIc61) corresponding to B-FcIc60 and B-FcIc61.

For the intein Gp41-1, the following plasmids were constructed: plasmids pA-HIn(70)˜pA-HIn(71) corresponding to A-Fab70 and A-Fab71, and plasmids pTag-Ic-FSb-(B-FcIc70) and pTag-Ic-FSb-(B-FcIc71) corresponding to B-FcIc70 and B-FcIc71.

For the intein Gp41-8, the following plasmids were constructed: plasmids pA-HIn(80)˜pA-HIn(81) corresponding to A-Fab80 and A-Fab81, and plasmids pTag-Ic-FSb-(B-FcIc80) and pTag-Ic-FSb-(B-FcIc81) corresponding to B-FcIc80 and B-FcIc81.

For the intein IMPDH-1, the following plasmids were constructed: plasmids pA-HIn(90)˜pA-HIn(92) corresponding to A-Fab90, A-Fab91 and A-Fab92, and plasmids pTag-Ic-FSb-(B-FcIc90)˜pTag-Ic-FSb-(B-FcIc92) corresponding to B-FcIc90˜B-FcIc92.

For the intein PhoRadA, the following plasmids were constructed: plasmids pA-HIn(100)˜pA-HIn(101) corresponding to A-Fab100 and A-Fab101, and plasmids pTag-Ic-FSb-(B-FcIc100) and pTag-Ic-FSb-(B-FcIc101) corresponding to B-FcIc100 and B-FcIc101.

The plasmids used in this Example to express the component A included: pA-HIn(20)˜(21), (30)˜(31), (40)˜(41), (50)˜(51), (60)˜(61), (70)˜(71), (80)˜(81), (90)˜(91), (100)˜(101), and pA-L.

The plasmids used in this Example to express the component B included: pTag-Ic-FSb-(B-FcIc20)˜21), (30)˜(31), (40)˜(41), (50)˜(51), (60)˜(61), (70)˜(71), (80)˜(81), (90)˜(91), (100)˜(101).

TABLE 34
Co-transfection pairings for inteins

| | | |
|---|---|---|
| Number | Component A | Component B |

| | | | |
|---|---|---|---|
| A21 | pA-HIn(81) | pA-L | pTag-Ic-FSb-(B-FcIc81) |
| A22 | pA-HIn(90) | pA-L | pTag-Ic-FSb-(B-FcIc90) |
| A23 | pA-HIn(50) | pA-L | pTag-Ic-FSb-(B-FcIc50) |
| A24 | pA-HIn(51) | pA-L | pTag-Ic-FSb-(B-FcIc51) |
| A25 | pA-HIn(70) | pA-L | pTag-Ic-FSb-(B-FcIc70) |
| A26 | pA-HIn(71) | pA-L | pTag-Ic-FSb-(B-FcIc71) |
| A27 | pA-HIn(80) | pA-L | pTag-Ic-FSb-(B-FcIc80) |
| A28 | pA-HIn(91) | pA-L | pTag-Ic-FSb-(B-FcIc91) |
| A29 | pA-HIn(51) | pA-L | pTag-Ic-FSb-(B-FcIc50) |
| A30 | pA-HIn(71) | pA-L | pTag-Ic-FSb-(B-FcIc70) |
| A31 | pA-HIn(81) | pA-L | pTag-Ic-FSb-(B-FcIc80) |
| A32 | pA-HIn(90) | pA-L | pTag-Ic-FSb-(B-FcIc91) |
| A33 | pA-HIn(50) | pA-L | pTag-Ic-FSb-(B-FcIc51) |
| A34 | pA-HIn(70) | pA-L | pTag-Ic-FSb-(B-FcIc71) |
| A35 | pA-HIn(80) | pA-L | pTag-Ic-FSb-(B-FcIc81) |
| A36 | pA-HIn(91) | pA-L | pTag-Ic-FSb-(B-FcIc90) |
| A37 | pA-HIn(30) | pA-L | pTag-Ic-FSb-(B-FcIc30) |
| A38 | pA-HIn(31) | pA-L | pTag-Ic-FSb-(B-FcIc31) |
| A39 | pA-HIn(31) | pA-L | pTag-Ic-FSb-(B-FcIc30) |
| A40 | pA-HIn(30) | pA-L | pTag-Ic-FSb-(B-FcIc31) |
| A41 | pA-HIn(60) | pA-L | pTag-Ic-FSb-(B-FcIc60) |
| A42 | pA-HIn(61) | pA-L | pTag-Ic-FSb-(B-FcIc61) |
| A43 | pA-HIn(61) | pA-L | pTag-Ic-FSb-(B-FcIc60) |
| A44 | pA-HIn(60) | pA-L | pTag-Ic-FSb-(B-FcIc61) |
| A45 | pA-HIn(20) | pA-L | pTag-Ic-FSb-(B-FcIc20) |
| A46 | pA-HIn(21) | pA-L | pTag-Ic-FSb-(B-FcIc21) |
| A47 | pA-HIn(21) | pA-L | pTag-Ic-FSb-(B-FcIc20) |
| A48 | pA-HIn(20) | pA-L | pTag-Ic-FSb-(B-FcIc21) |
| A49 | pA-HIn(40) | pA-L | pTag-Ic-FSb-(B-FcIc40) |
| A50 | pA-HIn(41) | pA-L | pTag-Ic-FSb-(B-FcIc41) |
| A51 | pA-HIn(41) | pA-L | pTag-Ic-FSb-(B-FcIc40) |
| A52 | pA-HIn(40) | pA-L | pTag-Ic-FSb-(B-FcIc41) |
| A53 | pA-HIn(100) | pA-L | pTag-Ic-FSb-(B-FcIc100) |
| A54 | pA-HIn(101) | pA-L | pTag-Ic-FSb-(B-FcIc101) |
| A55 | pA-HIn(101) | pA-L | pTag-Ic-FSb-(B-FcIc100) |
| A56 | pA-HIn(100) | pA-L | pTag-Ic-FSb-(B-FcIc101) |
| A58 | pA-HIn(92) | pA-L | pTag-Ic-FSb-(B-FcIc90) |
| A59 | pA-HIn(92) | pA-L | pTag-Ic-FSb-(B-FcIc92) |

Transfections were performed based on the pairs of Table 34. The transfection conditions were as follows: the molar ratio of plasmids was pTag-Ic-FSb(XX or XXX)-(B-FcIc): pA-HIn(XX or XXX): pA-L=3:1:1. And the transient transfection of monoclonal antibody was set as a positive control.

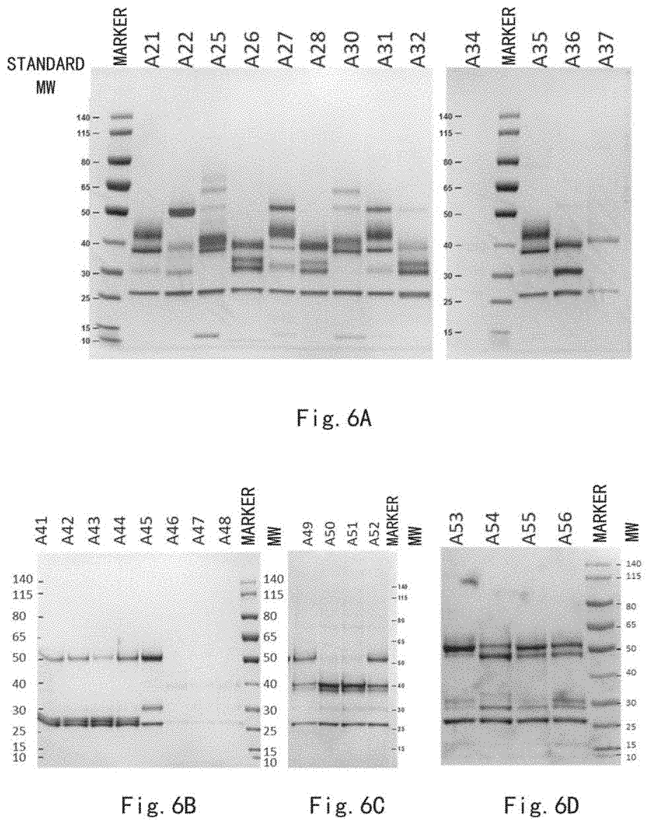

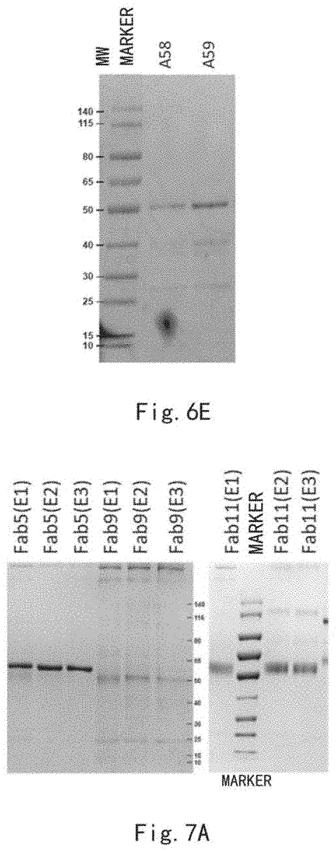

The transfected cells were cultured for 5 days and the supernatant was taken. Protein A affinity chromatography was performed on the proteins in the supernatant, and then a coomassie brilliant blue staining was performed by SDS-PAGE method (adding a reducing agent) to detect the proteins in the supernatant. The results were shown in FIGS. 6A to 6D, As can be seen from the result, groups A22, A27, A31, A45, A49, A52, A53, A55, and A56 show a significant splicing.

As can be seen from the result of FIG. 6E, groups A58 and A59 show a significant splicing.

The inteins and flanking sequences corresponding to groups A22, A27, A31, A45, A49, A52, A53, A55, A56, A58 and A59 are shown in Table 35.

TABLE 35
Different inteins and corresponding
effective flanking sequence pairs

| Intein | Number | Flanking sequence a | Flanking sequence b |
|---|---|---|---|
| IMPDH-1 | A22 | GGG | SI |
| IMPDH-1 | A58 | DKG | SI |
| IMPDH-1 | A59 | DKG | ST |
| Gp41-8 | A27 | NR | SAV |
| Gp41-8 | A31 | DK | SAV |
| SSpDnaB | A45 | SG | SIE |
| MjaTFIIB | A49 | TY | TIH |
| MjaTFIIB | A52 | TY | THT |
| PhoRadA | A53 | GK | TQL |
| PhoRadA | A55 | GK | THT |
| PhoRadA | A56 | DK | TQL |

In summary, the results show that for the intein IMPDH-1, the corresponding flanking sequence pair with excellent splicing efficiency is: when the flanking sequence a is GGG, the flanking sequence b is SI; or when the flanking sequence a is DKG, the flanking sequence b is ST; or when the flanking sequence a is DKG, the flanking sequence b is SI.

For the intein Gp41-8, the corresponding flanking sequence pair with excellent splicing efficiency is: when the flanking sequence a is NR, the flanking sequence b is SAV; or when the flanking sequence a is DK, the flanking sequence b is SAV.

For the intein SSpDnaB, the corresponding flanking sequence pair with excellent splicing efficiency is: when the flanking sequence a is SG, the flanking sequence b is SIE.

For the intein MjaTFIIB, the corresponding flanking sequence pair with excellent splicing efficiency is: when the flanking sequence a is TY, the flanking sequence b is TIH; or when the flanking sequence a is TY, the flanking sequence b is THT.

For the intein PhoRadA, the corresponding flanking sequence pair with excellent splicing efficiency is: when the flanking sequence a is GK, the flanking sequence b is TQL or THT; or when the flanking sequence a is DK, the flanking sequence b is TQL.

### EXAMPLE 3

### Intein-Mediated In Vitro Splicing of Polypeptide Fragments from Different Protein Sources

Construction of Vectors and Expression of Polypeptides

Under the same condition as that in Example 1, component expression plasmids of inteins SspDnaB, MxeGyrA, MjaTFIIB, PhoVMA, TVoVMA, Gp41-1, Gp41-8, IMPDH-1, PhoRadA were respectively constructed by pcDNA3.1 based on the structure as shown in Tables 31 and 33.

For the same component B′, the above component expression plasmids were averagely divided into three types: B′-L expression plasmid (pB′-L), B′-H expression plasmid (pB′-H) and B′-FcIc expression plasmid (pB′-FcIc); wherein, each component B′ shared the same pB′-L and B′-H expression plasmids.

For the intein SspDnaB, plasmids pB′-FcIc(20)˜B′-FcIc(21) corresponding to B′-HAb20˜B′-HAb21 were constructed.

For the intein MxeGyrA, plasmids pB′-FcIc(30)˜B′-FcIc(31) corresponding to B′-HAb30˜B′-HAb31 were constructed.

For the intein MjaTFIIB, plasmids pB′-FcIc(40)˜B′-FcIc(41) corresponding to B′-HAb40˜B′-HAb41 were constructed.

For the intein PhoVMA, plasmids pB′-FcIc(50)˜B′-FcIc(51) corresponding to B′-HAb50˜B′-HAb51 were constructed.

For the intein TVoVMA, plasmids pB′-FcIc(60)˜B′-FcIc(61) corresponding to B′-HAb60˜B′-HAb61 were constructed.

For the intein Gp41-1, plasmids pB′-FcIc(70)˜B′-FcIc(71) corresponding to B′-HAb70˜B′-HAb71 were constructed.

For the intein Gp41-8, plasmids pB′-FcIc(80)˜B′-FcIc(81) corresponding to B′-HAb80˜B′-HAb81 were constructed.

For the intein IMPDH-1, plasmids pB′-FcIc(90)˜B′-FcIc(92) corresponding to B′-HAb90˜B′-HAb92 were constructed.

For the intein PhoRadA, plasmids pB′-FcIc(100)˜B′-FcIc(101) corresponding to B′-HAb100˜B′-HAb101 were constructed.

The plasmids used in this Example to express the component A included: pA-HIn(90), pA-HIn(80), pA-HIn(81), pA-HIn(61), pA-HIn(20), pA-HIn(40), pA-HIn(100) and pA-L.

The plasmids used in this Example to express the component B′ included: pB′-FcIc(90), pB′-FcIc(80), pB′-FcIc(61), pB′-FcIc(20), pB′-FcIc(41), pB′-FcIc(101) and pB′-L, pB′-H.

Expression and purification of component A:

Each plasmid pA-HIn and the plasmid pA-L were co-transfected into CHO cells and cultured at 37° C., with a plasmid molar ratio of pA-HIn:pA-L=1:1, and the cell supernatant was harvested at 10 day after transfection. The supernatant was purified by nickel column chromatography (Jiangsu Qianchun, cat no. A41002-06) to obtain a purified polypeptide fragment of component A.

Expression and purification of component B′:

The plasmid pB′-L, plasmid pB′-H and each plasmid pB′-FcIc were co-transfected into 293E cells and cultured at 37° C., with a plasmid molar ratio of pB′-L:pB′-H:pB′-FcIc=1:1:3, and the cell supernatant was harvested at 10 day after transfection. The supernatant was purified by nickel column chromatography to obtain a purified polypeptide fragment of component B′.

As shown in Table 36, the obtained polypeptide fragments of component A and component B′ were referred to as Fab5˜Fab 11 and HAb5˜HAb11, respectively.

TABLE 36
The obtained polypeptide fragments
of component A and component B′

| | Corresponding | | Corresponding |
|---|---|---|---|
| Number of | plasmid of | Number of | plasmid of |
| component A | component A | component B′ | component B′ |
| Fab5 | pA-HIn(90) | HAb5 | pB′-L |
| | pA-L | | pB′-H |
| | | | pB′-FcIc(90) |
| Fab6 | pA-HIn(80) | HAb6 | pB′-L |
| | pA-L | | pB′-H |
| | | | pB′-FcIc(80) |
| Fab7 | pA-HIn(81) | HAb7 | pB′-L |
| | pA-L | | pB′-H |
| | | | pB′-FcIc(80) |
| Fab8 | pA-HIn(61) | HAb8 | pB′-L |
| | pA-L | | pB′-H |
| | | | pB′-FcIc(61) |
| Fab9 | pA-HIn(20) | HAb9 | pB′-L |
| | pA-L | | pB′-H |
| | | | pB′-FcIc(20) |
| Fab10 | pA-HIn(40) | HAb10 | pB′-L |
| | pA-L | | pB′-H |
| | | | pB′-FcIc(41) |
| Fab11 | pA-HIn(100) | HAb11 | pB′-L |
| | pA-L | | pB′-H |
| | | | pB′-FcIc(101) |

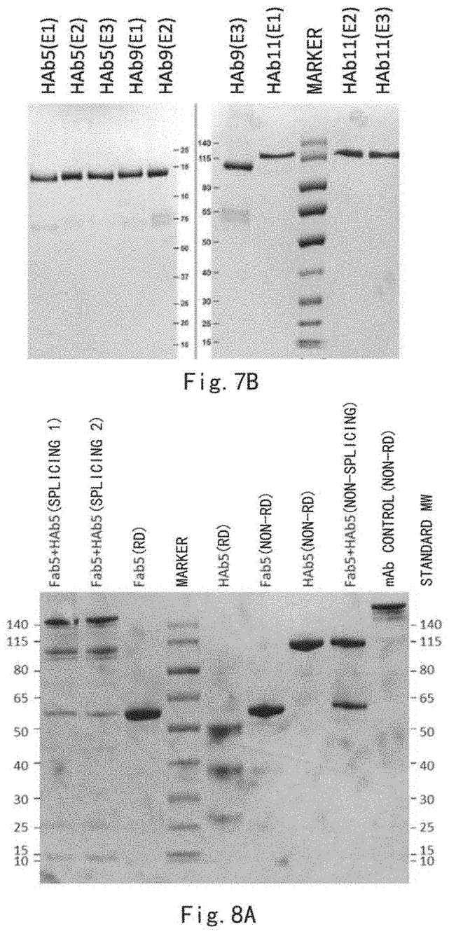

The obtained purified polypeptide fragments of component A and component B′ were subjected to non-reducing SDS-PAGE and coomassie brilliant blue staining, and the results were shown in FIGS. 7A to 7B.

E1, E2, and E3 represent elution fractions eluted with different imidazole concentrations (from low to high concentration) during nickel column chromatography. It can be seen from FIG. 7A that both Fab5 and Fab11 are expressed at a high level. Moreover, in the Fab5 and Fab11 groups, polypeptides with a high purity can be obtained by purifying with nickel column chromatography. It can be seen from FIG. 7B that HAb5, HAb9 and HAb11 are all expressed at a high level, and polypeptides with a higher purity can be obtained after HAb5, HAb9 and Hab11 being subjected to nickel column chromatography.

In Vitro Splicing

The obtained purified polypeptide fragments of component A and component B′, Fab5, Fab11, HAb5 and HAb11, were dialyzed into a buffer at 4° C. with a 31(D dialysis bag (purchased from Sigma), with a concentration of 1 to 10 micromolar. The buffer contained 10 to 50 mM Tris/HCl (pH 7.0-8.0), 100 to 500 mM NaCl, and 0 to 0.5 mM EDTA. Then, the components A and B′ with the same intein source were respectively mixed according to corresponding serial numbers thereof (for example, Fab5 and HAb5, etc.) at a molar ratio of 1:5 to 5:1, and DTT was added to be 0.5 to 5 mM, then the mixture was incubated overnight at 37° C.

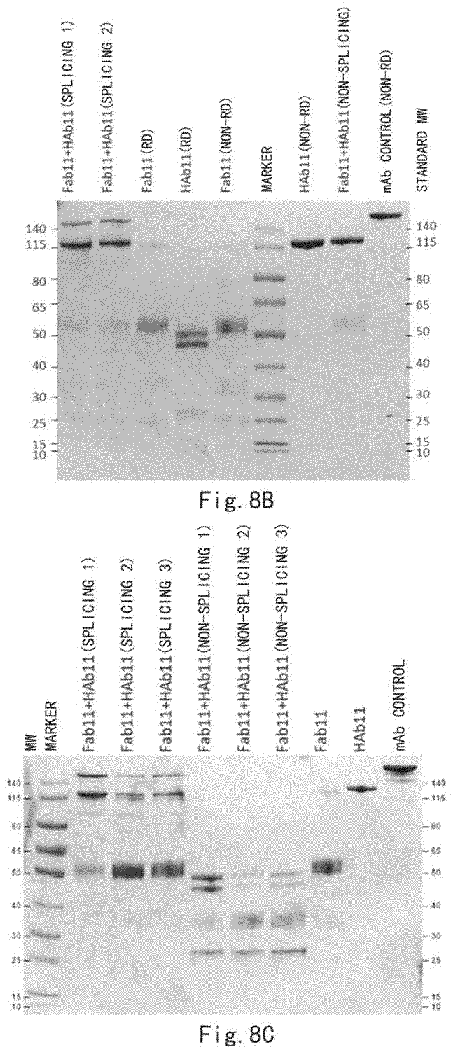

The obtained spliced product polypeptides were subjected to SDS-PAGE and coomassie brilliant blue staining, and the results were shown in FIGS. 8A to 8C.

In FIGS. 8A to 8B, “SPLICING 1” shows the result of a reaction system obtained by mixing component A and component B′ firstly, and then adding 2 mM DTT; “SPLICING 2” shows the result of a reaction system obtained by adding 2 mM DTT to component A and component B′ respectively, and then mixing the two; “reduced (i.e., RD)” means that the component contains 2 mM DTT, “non-reduced (i.e., NON-RD)” means that the component does not contain DTT; “NON-SPLICING ” means no DTT is added to the solution; the monoclonal antibody is Herceptin (purchased from Roche).

In FIG. 8C, “SPLICING 1” and “NON-SPLICING 1” show the results of reaction systems containing the component A and component B′ at concentrations of 5 μM and 4 μM, respectively, as well as 2 mM DTT; “SPLICING 2” and “NON-SPLICING 2” show the results of reaction systems containing the component A and component B′ with concentrations of 10 μM and 1 μM, respectively, as well as 2 mM DTT; “SPLICING 3” and “NON-SPLICING 3” show the results of reaction systems containing the component A and component B′ with concentrations of 5 μM and 1 μM, respectively, as well as 2 mM DTT; wherein “SPLICING 1” to “SPLICING 3” are incubated overnight at 37° C., and “NON-SPLICING 1” to “NON-SPLICING 3” are incubated at 4° C. overnight; the control bands are Fab11 (non-reduced, i.e., NON-RD) for component A, and HAb 11 (non-reduced, i.e., NON-RD) for component B′, and mAb.

It can be seen from FIG. 8 that the two split inteins IMPDH-1 and PhoRadA with the novel flanking sequence pair of the present disclosure have a high efficiency in effective splicing in vitro, thereby obtaining in vitro spliced recombinant polypeptides derived from polypeptide fragments of different proteins (i.e., spliced products Fab5+HAb5 and Fab11+HAb11, respectively). The band size of these spliced products are the same as that of the monoclonal antibody control (150 kD), demonstrating that the theoretical molecular weight of the product is consistent with that of natural IgG monoclonal antibody.

Biological Activity Detection of Spliced Product

The biological activity detection based on double antigen sandwich ELISA was performed for the recombinant polypeptide Fab5+HAb5 (SPLICING 1). The steps were as follows: 1) Preparation of antigen: for the proteins PD-L1 and CD38, only the extracellular domain was selected for construction, and an expression plasmid with His-tag was constructed by using the vector pcDNA3.1.

After construction, 293E cells were used for transient transfection, and a two-step purification including nickel column purification and molecular sieve purification was carried out. After purification, an antigen protein with a purity of no less than 95% detected by SDS-PAGE was obtained.

PD-L1 protein was labeled with horseradish peroxidase (HRP).

2) Coating of the first antigen: the concentration of CD38 protein was adjusted to 2 μg/ml, and an microtiter plate was coated with the CD38 protein-containing liquid at 100 μl/well, 4° C. overnight; the supernatant was discarded and 250 μl blocking solution (3% BSA in PBS) was added to each well;

3) addition of antibody: according to the experimental design, the operation was performed at room temperature. The antibody was diluted in a gradient with 1% BSA in PBS. For example, the initial concentration of antibody for dilution was 20 μg/mL, and the antibody was diluted by 2-fold with 5 gradients. The diluted antibody was added into wells of microtiter plate at 200 μl/well, incubated at room temperature for 2 hours, and then the supernatant was discarded;

4) washing: the plate was washed by 200 μl/well PBST (PBS containing 0.1% Tween20) for 3 times;

5) incubation of secondary antigen: a diluted secondary antigen (HRP-labeled PD-L1 protein) was added with a volume of 100 μl/well and incubated at room temperature for 1 hour, wherein the secondary antigen was diluted at 1:1000 and the diluent was 1% BSA in PBS;

6) washing: the plate was washed with 200 μl/well PBST for 5 times;

7) color-developing: TMB color-developing solution (prepared from A and B color-developing solutions purchased from Wuhan Boster Company, and mixed according to A:B=1:1, ready to use) was added at 100 μl/well, and the color-developing was performed at 37° C. for 5 min;

8) 2M HCl stopping solution was added at 100 μl/well, and then the microplate reader should be read at 450 nm within 30 minutes.

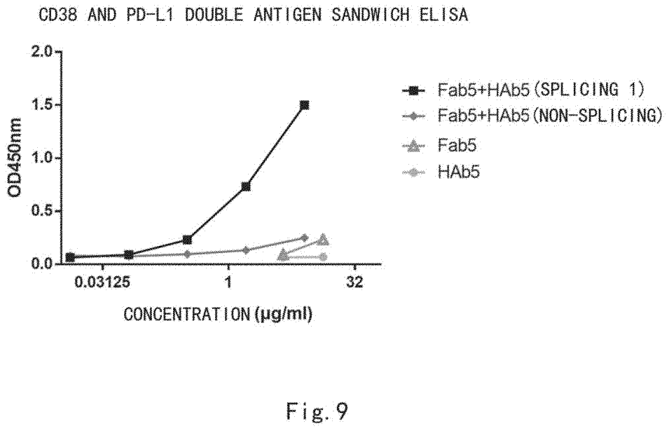

FIG. 9 shows the ELISA results of Fab5 polypeptide fragment, HAb5 polypeptide fragment, unspliced mixture of Fab5 and HAb5, and Fab5+HAb5 polypeptide fragment obtained by splicing Fab5 and HAb5 via the intein in vitro.

It can be seen from FIG. 9 that the Fab5+HAb5 (SPLICING 1) has the activity of simultaneously binding to both CD38 and PD-L1 antigens. The in vitro unspliced mixture, and the component A (Fab5) and component B (HAb5) alone, does not have the activity of simultaneously binding to both antigens.

The results prove that the spliced product Fab5+HAb5 (SPLICING 1) obtained by using the intein and the novel flanking sequence pair contained therein of the present disclosure has a good bispecific antibody activity.

Peptide Map Overlay Detection of Spliced Products

Peptide coverage refers to the ratio of the number of detected peptide amino acids to the total number of protein amino acids.

The detection of peptide coverage of a protein product is of great significance for confirming the primary amino acid sequence of protein drugs, ensuring the formation of higher-order structures of protein drugs and maintaining the properties of protein drugs. At present, the detection of protein peptide coverage is carried out by mass spectrometry according to the requirements of drug declaration. The detection of peptide coverage can be completed quickly, accurately and efficiently.

The peptide coverage of the protein Fab5+HAb5 (spliced product 1) was analyzed in this Example. The protein Fab5+HAb5 (spliced product 1) was digested by trypsin, chymotrypsin and Glu-C enzyme respectively, and the digested peptide samples were then analyzed by LC-MS/MS (XevoG2-XS QTof, waters). The LC-MS/MS data was analyzed by UNIFI (1.8.2, Waters) software, and the peptide coverage of Fab5+HAb5 (spliced product 1) was determined according to the algorithm results.

Experimental Apparatus:

1) High resolution mass spectrometer: XevoG2-XS QTof (Waters)

2) Ultra-high performance liquid chromatography: UPLC (Acquity UPLC I-Class) (Waters)

Materials and Reagents:

1) Guanidine HCl (Sigma)

2) Urea (Bio-Rad)

3) Tris-base (Bio-Rad)

4) DTT (Bio-Rad)

5) IAM (Sigma)

6) Zeba Spin column (Pierce)

7) ACQUITY UPLC CSH C18 Column, 130 Å, 1.7 μm, 2.1 mm×150 mm (Waters)

8) UNIFI (Waters)

9) Trypsin (Promega)

10) Chymotrypsin (Sigma)

11) Glu-C enzyme (Wako)

Experimental Method

1) Digestion with trypsin, chymotrypsin and Glu-C enzyme: the trypsin, chymotrypsin and Glu-C enzyme were added respectively to an appropriate amount of Fab5+HAb5 (splicing 1) after appropriate pretreatment and then digested at 37° C. for 20 hours.

2) High performance liquid chromatography: after digestion, the Fab5+HAb5 (spliced product 1) was separated by a ultra-high performance liquid chromatography system, Acquity UPLC I-Class, with a liquid phase A solution of 0.1% FA aqueous solution and a liquid phase B solution of 0.1% FA acetonitrile solution. The Fab5+HAb5 (spliced product 1) was loaded into the column by a autosampler, and then separated by the chromatographic column, with a column temperature of 55° C., a flow rate of 300 μl/min, and a 214 nm wavelength of TUV detector. The relevant liquid phase gradients were shown in Table 37.

TABLE 37
The ratio of solutions A and B in high
performance liquid chromatography

| | | Solution A | Solution B |
|---|---|---|---|
| | Time/min | percentage (%) | percentage (%) |
| 1 | 3 | 98 | 2 |
| 2 | 63 | 60 | 40 |
| 3 | 63.1 | 2 | 98 |
| 4 | 66 | 2 | 98 |
| 5 | 66.1 | 98 | 2 |
| 6 | 75 | 98 | 2 |

3) Mass spectrometry identification: the Fab5+HAb5 (spliced product 1) was detected and analyzed by XevoG2-XS QTof mass spectrometer (Waters) after being desalted and separated by the ultra-high performance liquid chromatography. Analysis time: 63 minutes; detection mode: positive ion, MS, scanning range (m/z): 300-2000.

4) Mass spectrometry data processing: the raw data were checked against the database by UNIFI (1.8.2, Waters) software, and the main parameters were as follows (Table 38):

TABLE 38
List of main parameters for mass spectrometry data
processing

| Item | Specific situation |
|---|---|
| Protease | Trypsin, chymotrypsin and Glu-C enzyme |
| protein | Glycosylated O-GN-G ST, Glycosylated O-GN-G-SA ST, |
| modification | Glycosylated O-G-SA ST, G0(N), G0F(N), G1F(N), G2F(N), |
| | Carbamidomethyl (C), Deamidated (NQ), Oxidation(M), Protein |
| | Terminal Acetyl (N-terminal) |
| M/Z | ±15 ppm |
| tolerance | |
| Fragment | ±20 ppm |
| tolerance | |
| theoretical | Light chain 1: |
| sequence of | DIQMTQSPSSLSASVGDRVTITCRASQDVSTAVAWYQQKPGKAPKLLIYSASFLYSGVPS |
| Fab5 + HAb5 | RFSGSGSGTDFTLTISSLQPEDFATYYCQQYLYHPATFGQGTKVEIKRTVAAPSVFIFPPSD |
| (spliced | EQLKSGTASVVCLLNNFYPREAKVQWKVDNALQSGNSQESVTEQDSKDSTYSLSSTLTL |
| product 1) | SKADYEKHKVYACEVTHQGLSSPVTKSFNRGEC (SEQ ID NO: 205) |
| | Heavy chain 1: |
| | EVQLVESGGGLVQPGGSLRLSCAASGFTFSDSWIHWVRQAPGKGLEWVAWISPYGGS |
| | TYYADSVKGRFTISADTSKNTAYLQMNSLRAEDTAVYYCARRHWPGGFDYWGQGTLVT |
| | VSSASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCGGGSICPPCPAPELL |
| | GGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVCTLP |
| | PSRDELTKNQVSLSCAVKGFYPSDIAVEWESNGQPENNYKTTPPVLDSDGSFFLVSKLTV |
| | DKSRWQQGNVFSCSVMHEALHNHYTQKSLSLSPGK (SEQ ID NO: 206) |
| | Heavy chain 2: |
| | EVQLLESGGGLVQPGGSLRLSCAVSGFTFNSFAMSWVRQAPGKGLEWVSAISGSGGG |
| | TYYADSVKGRFTISRDNSKNTLYLQMNSLRAEDTAVYFCAKDKILWFGEPVFDYWGQG |
| | TLVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQV |
| | YTLPPCRDELTKNQVSLWCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 207) |
| | Light chain 2: |
| | EIVLTQSPATLSLSPGERATLSCRASQSVSSYLAWYQQKPGQAPRLLIYDASNRATGIPAR |
| | FSGSGSGTDFTLTISSLEPEDFAVYYCQQRSNWPPTFGQGTKVEIKRTVAAPSVFIFPPSD |
| | EQLKSGTASVVCLLNNFYPREAKVQWKVDNALQSGNSQESVTEQDSKDSTYSLSSTLTL |
| | SKADYEKHKVYACEVTHQGLSSPVTKSFNRGEC (SEQ ID NO:208) |
| Filter | Minimum fragmentions: 3 |

Experimental Results and Analysis

The peptide samples obtained after digesting Fab5+HAb5 (spliced product 1) with trypsin, chymotrypsin and Glu-C enzyme respectively were analyzed by LC-MS/MS. The obtained raw data were checked against the database by UNIFI software. The database used was the theoretical sequence of Fab5+HAb5 (spliced product 1) provided by the customer.

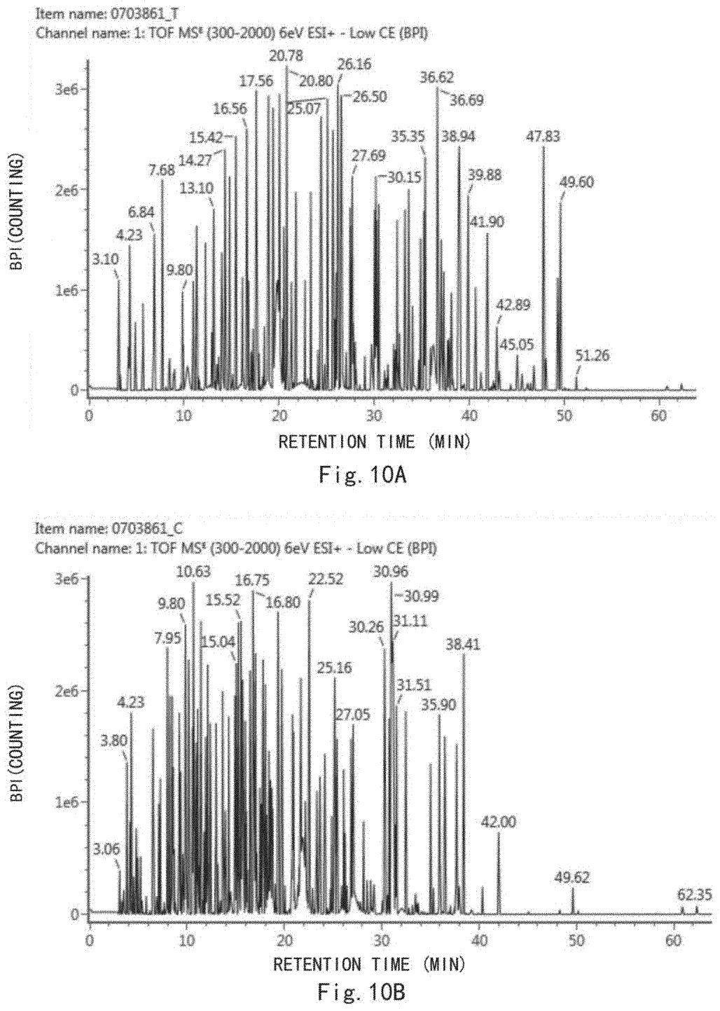

1) The BPI map after digestion of Fab5+HAb5 (spliced product 1) was shown in FIGS. 10A to 10C.

2) The coverage percentage after digestion by trypsin, chymotrypsin, and Glu-C enzyme were:

after trypsin digestion, the coverage percentage was 100%;

after chymotrypsin digestion, the coverage percentage was 100%;

after Glu-C enzyme digestion, the coverage percentage was 100%.

The digested samples were analyzed by LC-MS/MS and the database search results were integrated, finally obtaining a 100.00% peptide coverage for the Fab5+HAb5 (splicing 1). Based on the splicing principle of intein, according to the molecular weight of spliced product obtained in the present disclosure and the results of double-antigen sandwich ELISA and peptide map coverage, it can be speculated that an effective bispecific antibody with a natural IgG-like structure was obtained in the present disclosure. The test results confirmed that the structure of the bispecific antibody was a heterodimeric IgG structure composed of two different heavy chains and two different light chains, rather than a mixture of homodimeric IgG structure composed of two identical heavy chains and two identical light chains.

### EXAMPLE 4

### Intein-Mediated In Vitro Splicing of Different IgG Subclasses (1) Sequence of Component A

As shown in Table 39, the sequences corresponding to component A of three different IgG subclasses were as follows:

TABLE 39
Sequences corresponding to component
A of human IgG2, IgG3 and IgG4

| | | Expression | | Corre- | |
|---|---|---|---|---|---|
| Human IgG | Poly- | plasmid | | sponding | SEQ |
| subclass | peptide | name | Domain | code | ID NO |
| IgG2 | Fab102 | pA-HIn(102) | VHa | 10A7 | 106 |
| component | | | CH1 | G2CH1 | 180 |
| A | | | flanking | FSa7 | 57 |
| | | | sequence a | | |
| | | | In | PhoRadA | 49 |
| | | | tag protein | His-tag | 24 |
| | | | | Strep-tag | 28 |
| | | PA-L(1) | VLa | 10A7 | 107 |
| | | | CL | Lc2 | 173 |
| IgG3 | Fab103 | pA-HIn(103) | VHa | 10A7 | 106 |
| component | | | CH1 | G3CH1 | 181 |
| A | | | flanking | FSa7 | 57 |
| | | | sequence a | | |
| | | | In | PhoRadA | 49 |
| | | | tag protein | His-tag | 24 |
| | | | | Strep-tag | 28 |
| | | PA-L(1) | VLa | 10A7 | 107 |
| | | | CL | Lc2 | 173 |
| IgG4 | Fab104 | pA-HIn(104) | VHa | 10A7 | 106 |
| component | | | CH1 | G4CH1 | 182 |
| A | | | flanking | FSa9 | 59 |
| | | | sequence a | | |
| | | | In | IMPDH-1 | 47 |
| | | | tag protein | His-tag | 24 |
| | | | | Strep-tag | 28 |
| | | PA-L(1) | VLa | 10A7 | 107 |
| | | | CL | Lc2 | 173 |

As shown in Table 40, the sequences corresponding to component B of the three different IgG subclasses were as follows:

TABLE 40
Sequences corresponding to component
B of human IgG2, IgG3 and IgG4

| | | Expression | | Corre- | |
|---|---|---|---|---|---|
| Human IgG | Poly- | plasmid | | sponding | SEQ |
| subclass | peptide | name | Domain | code | ID NO |
| IgG2 | FcIc102 | pTag-Ic- | tag protein | Strep-tag | 28 |
| component | | FSb-(B- | | His-tag | 24 |
| B | | FcIc102) | Ic | PhoRadA | 50 |
| | | | flanking | FSb10 | 73 |
| | | | sequence b | | |
| | | | hinge | Hin2 | 169 |
| | | | Pb | G2CH2 | 184 |
| | | | | G2CH3 | 189 |
| IgG3 | FcIc103 | pTag-Ic- | tag protein | Strep-tag | 28 |
| component | | FSb-(B- | | His-tag | 24 |
| B | | FcIc103) | Ic | PhoRadA | 50 |
| | | | flanking | FSb10 | 73 |
| | | | sequence b | | |
| | | | hinge | Hin3 | 170 |
| | | | Pb | G3CH2 | 186 |
| | | | | G3CH3 | 190 |
| IgG4 | FcIc104 | pTag-Ic- | tag protein | Strep-tag | 28 |
| component | | FSb-(B- | | His-tag | 24 |
| B | | FcIc104) | Ic | IMPDH-1 | 48 |
| | | | flanking | FSb9 | 72 |
| | | | sequence b | | |
| | | | hinge | Hin4 | 171 |
| | | | Pb | G4CH2 | 187 |
| | | | | G4CH3 | 191 |

Transfections were performed based on the pairs of Table 41 in the same manner as that in Example 2. The transfection conditions were as follows: the molar ratio of plasmids was pTag-Ic-FSb-(B-FcIcxxx):pA-HIn(xxx):pA-L(1)=3:1:1. A positive control monoclonal antibody was also set as described above.

TABLE 41
Co-transfection pairing table for inteins
for expression of different IgG subclasses

| | | |
|---|---|---|
| Number | Component A | Component B |

| | | | |
|---|---|---|---|
| A102 | pA-HIn(102) | PA-L(1) | pTag-Ic-FSb-(B-FcIc102) |
| A103 | pA-HIn(103) | PA-L(1) | pTag-Ic-FSb-(B-FcIc103) |
| A104 | pA-HIn(104) | PA-L(1) | pTag-Ic-FSb-(B-FcIc104) |

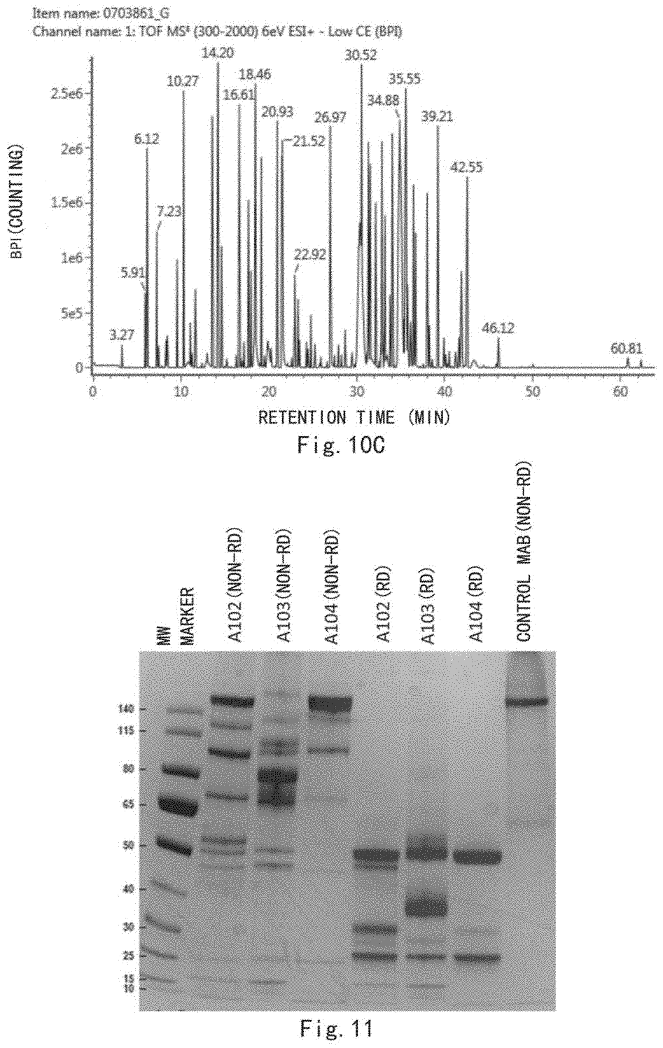

The transfected cells were cultured for 5 days and the supernatant was taken. Protein A affinity chromatography was performed on the proteins in the supernatant, and then a coomassie brilliant blue staining was performed by SDS-PAGE method (adding a reducing agent) to detect the proteins in the supernatant. The results were shown in FIG. 11.

As can be seen from the results, there was a significant splicing in human IgG2, IgG3 and IgG4 subclasses by using the intein; wherein, the A102 showed the intracellular expression by applying the intein PhoRadA to the component A and component B of human IgG2 subclass, indicating that an intact IgG2 mAb was formed by intracellular splicing; A103 showed the intracellular expression by applying the intein PhoRadA to the component A and component B of human IgG3 subclass, indicating that an intact IgG3 mAb was formed by intracellular splicing; A104 showed the intracellular expression by applying the intein IMPDH-1 to the component A and component B of human IgG4 subclass, indicating that an intact IgG4 mAb was formed by intracellular splicing.

### EXAMPLE 5

### Intein-Mediated In Vitro Splicing of Green Fluorescent Protein

The green fluorescent protein was EGFP (source: UniProtKB—A0A076FL24), and its full-length amino acid sequence was SEQ ID No: 23, with a total of 239 amino acid residues. The sequence was split into component A and component B; wherein (1) the component A was a fusion of amino acids at positions 1-158 of EGFP and an intein, and the corresponding coding DNA was constructed into an eukaryotic expression vector pcDNA3.1, with the flanking sequence a, the N′ fragment of intein (In) and a stop codon (TAA, TGA or TAG) added to the C-terminus, and the names of the constructed expression plasmids were shown in Table 42; (2) the component B was a fusion of amino acids at positions 159-239 of EGFP and an intein, and the corresponding coding DNA was constructed into an eukaryotic expression vector pcDNA3.1, with a start codon ATG, the C′fragment of intein (Ic) and the flanking sequence b added to its N-terminus, as well as with a stop codon (TAA, TGA or TAG) added to the C-terminus, and the names of the constructed expression plasmids were shown in Table 43. In addition, the EGFP full-length protein-encoding DNA was constructed into pcDNA3.1 (with a stop codon), and the plasmid was referred to as pEGFP.

TABLE 42
Names of the expression plasmid for component A of EGFP

| Plasmid | | | |
|---|---|---|---|
| name | Pa | Flanking sequence a | In |
| pGFP-N1 | N-terminus | DK (SEQ ID No: 60) | Gp41-8 (SEQ ID No: 45) |
| pGFP-N2 | of EGFP | DKG (SEQ ID No: 202) | IMPDH-1 (SEQ ID No: 47) |
| pGFP-N3 | (amino acids | GK (SEQ ID No: 57) | TvoVMA (SEQ ID No: 41) |
| pGFP-N4 | at positions | SG (SEQ ID No: 52) | SpDnaB (SEQ ID No: 33) |
| pGFP-N5 | 1-158 of SEQ | GK (SEQ ID No: 57) | PhoRadA (SEQ ID No: 49) |
| | ID No: 23) | | |

TABLE 43
Names of the expression plasmid for component B of EGFP

| Plasmid | | | |
|---|---|---|---|
| name | Ic | Flanking sequence b | Pb |
| pGFP-C1 | Gp41-8 (SEQ ID No: 46) | SAV (SEQ ID No: 71) | C-terminus |
| pGFP-C2 | IMPDH-1 (SEQ ID No: 48) | SI (SEQ ID No: 72) | of EGFP |
| pGFP-C3 | TvoVMA (SEQ ID No: 42) | THT (SEQ ID No: 77) | (amino acids |
| pGFP-C4 | SpDnaB (SEQ ID No: 34) | SIE (SEQ ID No: 66) | at positions |
| pGFP-C5 | PhoRadA (SEQ ID No: 50) | THT (SEQ ID No: 77) | 159 to 239 of SEQ |
| | | | ID No: 23) |

Based on the method of Example 1, the plasmids pEGFP-A and pEGFP were separately transfected or co-transfected into 293 cells or CHO cells with a co-transfection ratio of 1:1. In addition, the pEGFP was separately transfected into 293 or CHO cells as a positive control. The concentration of each plasmid was the same for separate transfection or co-transfection. 48 hours after transfection, the green fluorescence expression of cells was detected by flow cytometer. and the results were shown in Table 44.

TABLE 44
Green fluorescence expression results in
293 cells 48 hours after transfection

| | Mean fluorescence | Fluorescent cell |
|---|---|---|
| Transfected plasmid | intensity | percentage |
| pEGFP | 1 × 10\hat{ }5 | 99% |
| pGFP-N1 + pGFP-C1 | 3 × 10\hat{ }4 | 57% |
| pGFP-N1 | 221 | 0.1% |
| pGFP-C1 | 105 | 0 |
| pGFP-N2 + pGFP-C2 | 9.9 × 10\hat{ }4 | 99% |
| pGFP-N2 | 277 | 0.1% |
| pGFP-C2 | 146 | 0 |
| pGFP-N3 + pGFP-C3 | 1 × 10\hat{ }4 | 47% |
| pGFP-N3 | 177 | 0 |
| pGFP-C3 | 133 | 0 |
| pGFP-N4 + pGFP-C4 | 7 × 10\hat{ }4 | 88% |
| pGFP-N4 | 321 | 0.2% |
| pGFP-C4 | 152 | 0 |
| pGFP-N5 + pGFP-C5 | 8 × 10\hat{ }4 | 95% |
| pGFP-N5 | 274 | 0.1% |
| pGFP-C5 | 106 | 0 |
| Blank control | 139 | 0 |

As can be seen from the above results, different inteins and flanking sequences can effectively splice the green fluorescent protein in cells and form a structure very similar to that of the original green fluorescent protein, thereby generating the green fluorescence. Separate expression of component A or component B cannot generate the green fluorescence.

### INDUSTRIAL APPLICABILITY

The present disclosure provides methods for preparing recombinant polypeptides, particularly bispecific antibodies, by using split inteins with novel flanking sequence pairs. The split inteins with novel flanking sequence pairs of the present disclosure can be widely used in the preparation of recombinant polypeptides in the fields of medicine and bioengineering, especially in the field of antibodies, especially in the preparation of bispecific antibodies. The bispecific antibody prepared by using the split inteins with novel flanking sequence pairs of the present disclosure does not have a non-natural domain, has a structure closely similar to that of natural antibody (IgA, IgD, IgE, IgG or IgM), and has a Fc domain. The bispecific antibody has a complete structure and good stability, and can retain or remove CDC (complement-dependent cytotoxicity) or ADCC (antibody-dependent cytotoxicity) or ADCP (antibody-dependent cellular phagocytosis) or FcRn (Fc receptor)-binding activity according to different IgG subclasses.

The bispecific antibody prepared by the method of the present disclosure has the following advantages: the bispecific antibody has a long half-life in vivo and low immunogenicity, and does not introduce any form of linkers; has an improved stability, and a reduced in vivo immune response. The bispecific antibody prepared by the method of the present disclosure has the same glycosylation modification as that of wild-type IgG, has better biological function, is more stable, and has a long half-life in vivo; the in vitro splicing method by using inteins can completely avoid the problems of heavy chain mismatch and light chain mismatch commonly found in traditional methods.

The preparation method for bispecific antibodies of the present disclosure can also be used to produce humanized bispecific antibodies and bispecific antibodies with complete human sequences. The sequence of such an antibody prepared by the method of the present disclosure is more similar to that of a human antibody, which can effectively reduce the immune response. The preparation method for bispecific antibodies of the present disclosure is not limited by antibody subclasses (IgG, IgA, IgM, IgD, IgE, and light chain κ and λ types) and can be used to construct any bispecific antibody.

1. A flanking sequence pair for a split intein, wherein,
the flanking sequence pair comprises: a flanking sequence a and a flanking sequence b; wherein, the flanking sequence a is located at N-terminus of a split intein N-terminal protein splicing region (In), and is between a N-terminal extein (En) and the In; the flanking sequence b is located at C-terminus of a split intein C-terminal protein splicing region (Ic), and is between the Ic and a C-terminal extein (Ec);
the split intein is selected from the group consisting of SspDnaE, SspDnaB, MxeGyrA, MjaTFIIB, PhoVMA, TVoVMA, Gp41-1, Gp41-8, IMPDH-1 or PhoRadA,
(1) when the split intein is IMPDH-1,
the flanking sequence a is A₋₃A₋₂A₋₁ and the flanking sequence b is B₁B₂B₃, wherein:
A₋₃ is X or deletion, or preferably G or D; A₋₂ is X or deletion, or preferably G or K; A₋₁ is selected from G or T;
B₁ is S; B₂ is I or T or S; B₃ is X or deletion;
preferably,
the flanking sequence a is G, XG, XGG, DKG or DKT, and the flanking sequence b is SI, ST, SS, SIX, STX or SSX;
(2) when the split intein is Gp41-8,
the flanking sequence a is A₋₃A₋₂A₋₁ and the flanking sequence b is B₁B₂B₃, wherein:
A₋₃ is X or deletion; A₋₂ is selected from N or D; A₋₁ is selected from R or K;
B₁ is S or T; B₂ is A or H; B₃ is X or deletion, or preferably V, Y or T,
preferably,
the flanking sequence a is NR, XNR, DK, XDK, DR or XDR, and the flanking sequence b is SA or SAX;
(3) when the split intein is SspDnaB,
the flanking sequence a is A₋₃A₋₂A₋₁ and the flanking sequence b is B₁B₂B₃, wherein:
A₋₃ is X or deletion; A₋₂ is selected from S or D; A₋₁ is selected from G or K;
B₁ is S; B₂ is I; B₃ is X or deletion, or preferably E or T,
preferably,
the flanking sequence a is SG, XSG, DK, XDK, and the flanking sequence b is SI or SIX;
(4) when the intein is MjaTFIIB,
the flanking sequence a is A₋₃A₋₂A₋₁, and the flanking sequence b is B₁B₂B₃, wherein
A₋₃ is X or deletion; A₋₂ is selected from T or D; A₋₁ is selected from Y;
B₁ is T; B₂ is I or H; B₃ is X or deletion, or preferably H or T;
preferably,
the flanking sequence a is TY, DY, XTY or XDY, and the flanking sequence b is TI, TIX, TH or THX;
(5) when the split intein is PhoRadA,
the flanking sequence a is A₋₃A₋₂A₋₁ and the flanking sequence b is B₁B₂B₃, wherein:
A₋₃ is X or deletion; A₋₂ is selected from G or D; A₋₁ is selected from K;
B₁ is T; B₂ is Q or H; B₃ is X or deletion, or preferably L or T,
preferably,
the flanking sequence a is GK, XGK, DK or XDK, and the flanking sequence b is TQ, TH, TQX or THX;
(6) when the split intein is TVoVMA,
the flanking sequence a is A₋₃A₋₂A₋₁ and the flanking sequence b is B₁B₂B₃, wherein:
A₋₃is X or deletion; A₋₂ is selected from G or D; A₋₁ is K;
B₁ is T; B₂ is V or H; B₃ is X or deletion, or preferably I or T,
preferably,
the flanking sequence a is GK, XGK, DK or XDK, and the flanking sequence b is TV, TH, TVX or THX;
(7) when the split intein is MxeGyrA,
the flanking sequence a is A₋₃A₋₂A₋₁ and the flanking sequence b is B₁B₂B₃, wherein:
A₋₃is X or deletion; A₋₂ is selected from R or D; A₋₁ is selected from Y, K or T;
B₁ is T; B₂ is E or H; B₃ is X or deletion, or preferably A or T,
preferably,
the flanking sequence a is RY, XRY, DK or XDK, and the flanking sequence b is TE, TH, TEX or THX;
(8) when the split intein is PhoVMA,
the flanking sequence a is A₋₃A₋₂A₋₁ and the flanking sequence b is B₁B₂B₃, wherein:
A₋₃ is X or deletion; A₋₂ is selected from G or D; A₋₁ is selected from K;
B₁ is T; B₂ is V or H; B₃ is X or deletion, or preferably I or T,
preferably,
the flanking sequence a is GK, XGK, DK or XDK, and the flanking sequence b is TV, TH, TVX or THX;
(9) when the split intein is Gp41-1,
the flanking sequence a is A₋₃A₋₂A₋₁ and the flanking sequence b is B₁B₂B₃, wherein:
A₋₃ is X or deletion; A₋₂ is selected from G or D; A₋₁ is selected from Y or K;
B₁ is S or T; B₂ is S or H; B₃ is X or deletion, or preferably S or T;
preferably,
the flanking sequence a is GY, XGY, DK or XDK, and the flanking sequence b is SS, SH, SSX or SHX;
(10) when the split intein is SspDnaE,
the flanking sequence a is A₋₃A₋₂A₋₁ and the flanking sequence b is B₁B₂B₃, wherein:
A₋₃is X or deletion; A₋₂ is selected from G or D; A₋₁ is selected from G, S or K;
B₁ is T or S; B₂ is E or H; B₃ is X or deletion, or preferably T;
preferably,
the flanking sequence a is GG, XGG, GK, XGK, DK or XDK, and the flanking sequence b is SE, TH, SEX or THX;
wherein the X is any amino acid selected from the group consisting of G, A, V, L, M, I, S, T, P, N, Q, F, Y, W, K, R, H, D, E, and C.

2. The flanking sequence pair for a split intein according to claim 1, wherein the split intein together with the flanking sequence pair are used for trans-splicing,
wherein,
the SspDnaE is composed of the In of sequence as SEQ ID NO:31 and the Ic of sequence as SEQ ID NO:32,
the SspDnaB is composed of the In of sequence as SEQ ID NO:33 and the Ic of sequence as SEQ ID NO:34,
the MxeGyrA is composed of the In of sequence as SEQ ID NO:35 and the Ic of sequence as SEQ ID NO:36,
the MjaTFIIB is composed of the In of sequence as SEQ ID NO:37 and the Ic of sequence as SEQ ID NO:38,
the PhoVMA is composed of the In of sequence as SEQ ID NO:39 and the Ic of sequence as SEQ ID NO:40,
the TvoVMA is composed of the In of sequence as SEQ ID NO:41 and the Ic of sequence as SEQ ID NO:42,
the Gp41-1 is composed of the In of sequence as SEQ ID NO:43 and the Ic of sequence as SEQ ID NO:44,
the Gp41-8 is composed of the In of sequence as SEQ ID NO:45 and the Ic of sequence as SEQ ID NO:46,
the IMPDH-1 is composed of the In of sequence as SEQ ID NO:47 and the Ic of sequence as SEQ ID NO:48,
the PhoRadA is composed of the In of sequence as SEQ ID NO:49 and the Ic of sequence as SEQ ID NO:50.

3. A recombinant polypeptide obtained by trans-splicing via the flanking sequence pair for a split intein according to claim 1.

4. The recombinant polypeptide according to claim 3, wherein the recombinant polypeptide is obtained by a component A and a component B through trans-splicing;
in the component A, the N-terminus of the flanking sequence a is connected to the C-terminus of the En, and the C-terminus of the flanking sequence a is connected to the In, optionally a tag protein is connected to the C-terminus of the In;
in the component B, the C-terminus of the flanking sequence b is connected to the N-terminus of the Ec, and the N-terminus of the flanking sequence b is connected to the Ic, optionally a tag protein is connected to the N-terminus of the Ic;
wherein, coding sequences of the En and the Ec are respectively derived from a N-terminal part and a C-terminal part of the same protein.

5. The recombinant polypeptide according to claim 3, wherein the recombinant polypeptide is obtained by a component A and a component B through trans-splicing;
in the component A, the N-terminus of the flanking sequence a is connected to the C-terminus of the En, and the C-terminus of the flanking sequence a is connected to the In, optionally a tag protein is connected to the C-terminus of the In;
in the component B, the C-terminus of the flanking sequence b is connected to the N-terminus of the Ec, and the N-terminus of the flanking sequence b is connected to the Ic, optionally a tag protein is connected to the N-terminus of the Ic;
wherein, coding sequences of the En and the Ec are derived from different proteins.

6. The recombinant polypeptide according to claim 4, wherein the recombinant polypeptide is a fluorescent protein, protease, signal peptide, antimicrobial peptide, antibody, or a polypeptide with biological toxicity.

7. The recombinant polypeptide according to claim 4, wherein the same protein, or one or more of the different proteins is an antibody.

8. The recombinant polypeptide according to claim 7, wherein the antibody is a natural immunoglobulin class IgG, IgM, IgA, IgD or IgE, or an immunoglobulin subclass: IgG1, IgG2, IgG3, IgG4, IgG5, or with light chains of different classes: kappa, lambda; or a single domain antibody; or
the antibody is a full-length antibody or a functional fragment of an antibody.

9. The recombinant polypeptide according to claim 8, wherein the functional fragment of an antibody is selected from one or more of the group consisting of: antibody heavy chain variable region VH, antibody light chain variable region VL, antibody heavy chain constant region fragment Fc, antibody heavy chain constant region 1 CH1, antibody heavy chain constant region 2 CH2, antibody heavy chain constant region 3 CH3, antibody light chain constant region CL or single domain antibody variable region VHH.

10. The recombinant polypeptide according to claim 7, wherein, the same protein or one or more of the different proteins is specific to an antigen or epitope A,
the antigen A comprises: tumor cell surface antigen, immune cell surface antigen, cytokine, cytokine receptor, transcription factor, membrane protein, actin, virus, bacteria, endotoxin, FIXa, FX, CD3, SLAMF7, CD38, BCMA, CD20, CD16, CEA, PD-L1, PD-1, CTLA-4, TIGIT, LAG-3, VEGF, B7-H3, Claudin18.2, TGF-β, Her2, IL-10, Siglec-15, Ras, C-myc, and the epitope A is an immunogenic epitope of the antigen A.

11. The recombinant polypeptide according to claim 10, wherein, the same protein or one or more of the different proteins is specific to an antigen or epitope B different from the antigen or epitope A,
the antigen B comprises: tumor cell surface antigen, immune cell surface antigen, cytokine, cytokine receptor, transcription factor, membrane protein, actin, virus, bacteria, endotoxin, FIXa, FX, CD3, SLAMF7, CD38, BCMA, CD20, CD16, CEA, PD-L1, PD-1, CTLA-4, TIGIT, LAG-3, VEGF, B7-H3, Claudin18.2, TGF-β, Her2, IL-10, Siglec-15, Ras, C-myc, and the epitope B is an immunogenic epitope of the antigen B.

12. The recombinant polypeptide according to claim 11, which is a bispecific antibody that can simultaneously bind to both the antigen or epitope A and the antigen or epitope B.

13. The flanking sequence pair according to claim 2, wherein:
(1) when the split intein is IMPDH-1, the flanking sequence a is XGG and the flanking sequence b is SI, ST, SS; or the flanking sequence a is DKG and the flanking sequence b is SI, ST, SS; or the flanking sequence a is DKT and the flanking sequence b is SI, ST, SS;
(2) when the split intein is Gp41-8, the flanking sequence a is NR and the flanking sequence b is SAV; or the flanking sequence a is DK and the flanking sequence b is SAV; the flanking sequence a is NR and the flanking sequence b is SAT; or the flanking sequence a is DK and the flanking sequence b is SAT;
(3) when the split intein is SspDnaB, the flanking sequence a is SG and the flanking sequence b is SIE;
(4) when the split intein is PhoRadA, the flanking sequence a is GK and the flanking sequence b is TQL or THT; or the flanking sequence a is DK and the flanking sequence b is TQL or THT;
(5) when the split intein is TVoVMA, the flanking sequence a is GK and the flanking sequence b is TVI or THT; or the flanking sequence a is DK and the flanking sequence b is TVI or THT;
(6) when the split intein is MxeGyrA, the flanking sequence a is RY and the flanking sequence b is TEA or THT; or the flanking sequence a is DK and the flanking sequence b is TEA or THT;
(7) when the split intein is MjaTFIIB, the flanking sequence a is TY and the flanking sequence b is TIH; or the flanking sequence a is TY and the flanking sequence b is THT;
(8) when the split intein is PhoVMA, the flanking sequence a is GK and the flanking sequence b is TVI or THT; or the flanking sequence a is DK and the flanking sequence b is TVI or THT;
(9) when the split intein is Gp41-1, the flanking sequence a is GY and the flanking sequence b is SSS or SHT; or the flanking sequence a is DK and the flanking sequence b is SSS or SHT;
(10) when the split intein is SspDnaE, the flanking sequence a is GG and the flanking sequence b is SET or THT; or the flanking sequence a is GK and the flanking sequence b is SET or THT; or the flanking sequence a is DK and the flanking sequence b is SET or THT;
wherein the X is any amino acid selected from the group consisting of G, A, V, L, M, I, S, T, P, N, Q, F, Y, W, K, R, H, D, E, C.

14. The recombinant polypeptide according to claim 4, wherein the tag protein is selected from the group consisting of SEQ ID NO: 24, 25, 26, 27, 28, 29 and 30.

15. The recombinant polypeptide according to claim 12, which is a humanized bispecific antibody or a bispecific antibody of complete human sequence.
